(19) United States
(12) Patent Application Publication
(10) Pub. No.: US 2024/0208997 A1
(43) Pub. Date: Jun. 27, 2024

(54) CYCLIC BENZIMIDAZOLE DERIVATIVES AS CGAS INHIBITORS

(71) Applicant: Boehringer Ingelheim International GmbH, Ingelheim am Rhein (DE)

(72) Inventors: Annekatrin Charlotte HEIMANN, Biberach an der Riss (DE); Alexander DREYER, Gutenzell-Huerbel (DE); Christian GNAMM, Biberach an der Riss (DE); Cédrickx GODBOUT, Schemmerhofen (DE); Marc Alexander GRUNDL, Biberach an der Riss (DE); Sandra Ruth HANDSCHUH, Biberach an der Riss (DE); Christoph HOENKE, Biberach an der Riss (DE); Joerg Thomas KLEY, Mittelbiberach (DE); Christian Andreas KUTTRUFF, Donaueschingen (DE); Jun LI, Danbury, CT (US); Dirk REINERT, Bad Duerkheim (DE); Raphael STUBER, Schwendi (DE)

(21) Appl. No.: 18/386,979

(22) Filed: Nov. 3, 2023

Related U.S. Application Data
(60) Provisional application No. 63/382,948, filed on Nov. 9, 2022.

(30) Foreign Application Priority Data
Dec. 9, 2022 (EP) 22212494.3

(51) Int. Cl. C07D 498/22; A61K 45/06; C07D 491/048

(52) U.S. Cl. CPC C07D 498/22; A61K 45/06; C07D 491/048

(57) ABSTRACT

The invention relates to compounds of formula I

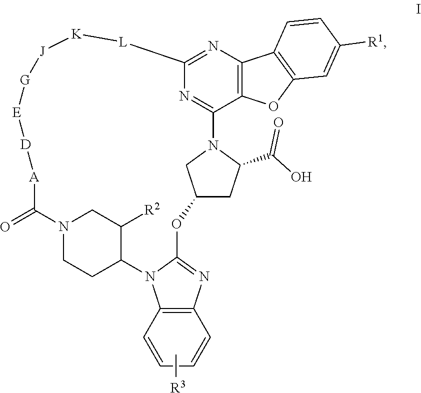

wherein R¹, R², R³, A, D, E, G, J, K and L are defined as in claim 1, and prodrugs, deuterated analogues and pharmaceutical acceptable salts thereof, for the treatment of diseases such as systemic lupus erythematosus, systemic sclerosis (SSc), interferonopathies, non-alcoholic steatohepatitis (NASH), interstitial lung disease (ILD) and idiopathic pulmonary fibrosis (IPF).

### 1. BACKGROUND OF THE INVENTION

### 1.1 cGAS Inhibitors

Innate immunity is considered a first line cellular stress response defending the host cell against invading pathogens and initiating signaling to the adaptive immune system. These processes are triggered by conserved pathogen-associated molecular patterns (PAMPs) through sensing by diverse pattern recognition receptors (PRRs) and subsequent activation of cytokine and type I interferon gene expression. The major antigen-presenting cells, such as monocytes, macrophages, and dendritic cells produce type I interferons and are critical for eliciting adaptive T- and B-cell immune system responses. The major PRRs detect aberrant, i.e. mislocalized, immature or unmodified nucleic acids on either the cell surface, the inside of lysosomal membranes or within other cellular compartments (Barbalat et al., Annu. Rev. Immunol. 29, 185-214 (2011)).

“Cyclic GMP-AMP Synthase” (cGAS, UniProtKB-Q8N884)) is the predominant sensor for aberrant double-stranded DNA (dsDNA) originating from pathogens or mislocalization or misprocessing of nuclear or mitochondrial cellular dsDNA (Sun et al., Science 339, 786-791 (2013); Wu et al., Science 339, 826-830 (2013); Ablasser et al., Nature 498, 380-384 (2013)). Binding of dsDNA to cGAS activates the reaction of GTP and ATP to form the cyclic dinucleotide GMP-AMP (referred to as cGAMP). cGAMP then travels to and activates the endoplasmatic reticulum membrane-anchored adaptor protein, “Stimulator of Interferon Genes” (STING). Activated STING recruits and activates TANK-binding kinase 1 (TBK1) which in turn phosporylates the transcription factor family of interferon regulatory factors (IRFs) inducing cytokine and type I interferon mRNA expression.

The critical role of cGAS in dsDNA sensing has been established in different pathogenic bacteria (Hansen et al., EMBOJ. 33, 1654 (2014)), viruses (Ma et al., PNAS 112, E4306 (2015)) and retroviruses (Gao et al., Science 341, 903-906 (2013)). Additionally, cGAS is essential in various other biological processes such as cellular senescence (Yang et al., PNAS 114, E4612 (2017), Gluck et al., Nat. Cell Biol. 19, 1061-1070 (2017)) and recognition of ruptured micronuclei in the surveillance of potential cancer cells (Mackenzie et al., Nature 548, 461-465 (2017); Harding et al., Nature 548, 466-470 (2017)).

While the cGAS pathway is important for host defense against invading pathogens, cellular stress and genetic factors may also cause production of aberrant cellular dsDNA, e.g. by nuclear or mitochondrial leakage, and thereby trigger autoinflammatory responses. Aicardi-Goutieres syndrome (AGS; Crow et al., Nat. Genet. 38, 917-920 (2006))—a lupus-like severe autoinflammatory immune-mediated disorder—arises from loss-of-function mutations in TREX1, a primary DNA exonuclease responsible for degrading aberrant DNA in cytosol. Knock-out of cGAS in TREX1-deficient mice prevented otherwise lethal autoimmune responses, supporting cGAS as driver of interferonopathies (Gray et al., J. Immunol. 195, 1939-1943 (2015); Gao et al., PNAS 112, E5699-E5705 (2015)). Likewise, embryonic lethality caused by deficiency of DNAse2, an endonuclease responsible for degradation of excessive DNA in lysosomes during endocytosis, was completely rescued by additional knock-out of cGAS (Gao et. al, PNAS 112, E5699-E5705 (2015)) or STING (Ahn et al., PNAS 109, 19386-19391 (2012)). These observations support cGAS as a drug target and inhibition of cGAS may provide a therapeutic strategy for preventing autoinflammation and treating diseases such as systemic lupus erythematosus (SLE) with involvement of anti-dsDNA antibodies (Pisetsky et al., Nat. Rev. Rheumatol. 12, 102-110 (2016)).

### 1.2 Prior Art

Due to the observation that inhibition of the cGAS-pathway may provide a therapeutic strategy for preventing autoinflammation and for treating e.g. autoimmune diseases many efforts to develop cGAS inhibitors have been undertaken.

In WO 2019/241787 for example, methyl 4-amino-6-(phenylamino)-1,3,5-triazine-2-carboxylates such as CU-32 and CU-76 have been disclosed as cGAS-inhibitors with “in vitro hcGAS IC₅₀-values” slightly below 1 μM (IC₅₀(CU-32)=0.66 μM and IC₅₀(CU-76=0.27 μM).

In Hall et al., PLoS ONE 12(9); e0184843 (2017), compound PF-06928215 has been published as an inhibitor of cGAS with an “in vitro hcGAS IC₅₀-value” of 0.049 μM as measured by a fluorescence polarization assay. However, compound PF-06928215 showed no acceptable cellular activity as a cGAS inhibitor.

In WO 2020/142729 and in WO2022/174012, (benzofuro[3,2-d]pyrimidin-4-yl)pyrrolidine-2-carboxylic acid derivatives have been disclosed as cGAS inhibitors for the therapy of autoimmune disorders such as Aicardi-Goutieres Syndrome (AGS), lupus erythematosus, scleroderma, inflammatory bowel disease and non-alcoholic steatohepatitis (NASH). However, the compounds of this invention differ from the (benzofuro[3,2-d]pyrimidin-4-yl)pyrrolidine-2-carboxylic acid derivatives of WO 2020/142729 in their completely different substitution pattern in the 4-position of the pyrrolidine ring.

Recently provided cGAS inhibitors, such as the ones in WO 2020/142729 or in WO 2022/174012, usually show an insufficient cellular cGAS inhibitory potency (with IC₅₀-values regarding inhibition of the cGAS/STING pathway as measured in cellular assays of usually larger than 1 μM, often of larger than 5 μM). However, it is crucial to provide therapeutic cGAS inhibitors that do not only show a satisfying biochemical (in vitro) inhibitory potency (“hcGAS IC₅₀”), but also a satisfying cellular inhibitory potency (for example by showing inhibition of IFN induction in virus-stimulated THP-1 cells (THP1₍ᵥᵢᵣ₎ IC₅₀)) in order to ensure that the compound is able to show a therapeutic effect in a patient.

Other important properties that may be predictive for successful development of a cGAS inhibitor as a therapeutic agent are satisfying cGAS-selectivity (versus off-target activity) and acceptable inhibitory potency in human whole blood.

Surprisingly it has now been found that the compounds of formula I, II or III show at the same time the following three properties:
a satisfying “biochemical (in vitro) IC₅₀-value regarding cGAS inhibition” (with a hcGAS IC₅₀ of 100 nM, preferably of 50 nM, in particular of 10 nM),
a satisfying “inhibition of IFN induction in virus-stimulated THP-1 cells (with a THP1 IC₅₀₍ᵥᵢᵣ₎ of ≤1 μM, preferably of 500 nM, more preferably of 100 nM, in particular of 50 nM) and
a satisfying selectivity for cGAS-inhibition (with a ratio THP1 IC_{50(cGAMP)}/THP1 IC₅₀₍ᵥᵢᵣ₎ of ≥10, more preferably ≥50, more preferably ≥500, in particular ≥1000).

Additionally the compounds of formula I, II or III also show acceptable IC₅₀-values with regard to inhibition of IFN induction in dsDNA-stimulated human whole blood assays, preferably with human whole blood IC₅₀-values with regard to cGAS inhibition (hWB IC₅₀) of 5000 nM, more preferably of 1000 nM, in particular of 100 nM.

The cGAS inhibitors of the invention with this particular pharmacological profile which combines an excellent in vitro inhibitory potency and an excellent cellular inhibitory potency with a high selectivity for cGAS inhibition have a high probability to also exhibit a good therapeutic effect in the patient. Due to their high cellular inhibitory potency compounds with this particular pharmacological profile should be able to pass the cell membrane barrier and therefore reach their intracellular target location and due to their selectivity to exclusively inhibit cGAS activity, these compounds should not show unwanted off target effects, for example side effects somewhere within the signaling pathway downstream of cGAS or cytotoxic effects.

### 2. DESCRIPTION OF THE INVENTION

The invention relates to a compound of formula I

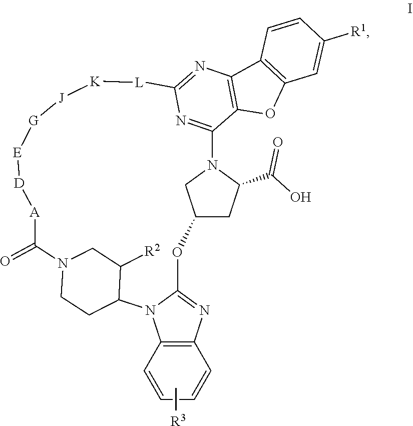

wherein
R¹ is selected from the group consisting of hydrogen, halogen, methyl, ethyl, —CF₃, —CHF₂, CFH₂ and methoxy,
R² is selected from the group consisting of hydrogen and methyl,
R³ is selected from the group consisting of hydrogen, methyl and halogen,
and wherein
A is selected from the group consisting of —CH₂—, —O—, —CF₂—, —CHF—, —N(CH₃)—, —NH— and —CHCH₃—;
D is selected from the group consisting of —CH₂—, —O—, —CF₂—, —CHF— and —CHCH₃—;
E is selected from the group consisting of —CH₂—, —O—, —C(CH₃)₂—, —CHF—, CF₂— and —CHCH₃—;
G is selected from the group consisting of —NH—, —NCH₃—, —CH₂—, —O—, —CF₂—, —CHF—, —CHCH₃— and —C(CH₃)₂;
J is selected from the group consisting of —CO—, —CH₂—, —O—, —CHF—, —CF₂— and —CHCH₃—;
K is selected from the group consisting of —CH₂—, —CF₂—, —O— or is absent;
L is selected from the group consisting of —CH₂—, —O—, —CHCH₃—, —CHF—, —CF₂— or is absent;
and prodrugs, deuterated analogues and pharmaceutical acceptable salts thereof.

Hereby variables A, D, E, G, J, K and L are preferably selected in such a way that two or more heteroatoms may not follow directly upon each other.

In a preferred embodiment the invention concerns the above-mentioned compound of formula I, wherein
R¹ is selected from the group consisting of hydrogen, Cl and F,
R² is selected from the group consisting of hydrogen and methyl,
R³ is selected from the group consisting of hydrogen, methyl, Cl and F,
and wherein
A is selected from the group consisting of —CH₂—, —O—, —CF₂—, —N(CH₃)—,
D is selected from the group consisting of —CH₂—, —O— and —CHCH₃—,
E is selected from the group consisting of —CH₂—, —O— and —C(CH₃)₂—,
G is selected from the group consisting of —NH—, —CH₂—, —O—, —CHCH₃—,
J is selected from the group consisting of —CO—, —CH₂—, —O—, and —CHCH₃—;
K is selected from the group consisting of —CH₂—, —CF₂—, —O— or is absent;
L is selected from the group consisting of —CH₂—, —CF₂— or is absent;
and prodrugs, deuterated analogues and pharmaceutical acceptable salts thereof.

Hereby variables A, D, E, G, J, K and L are preferably selected in such a way that two or more heteroatoms may not follow directly upon each other.

In a further preferred embodiment the invention relates to the above-mentioned compound of formula I, wherein L is absent, and prodrugs, deuterated analogues and pharmaceutical acceptable salts thereof.

In a further preferred embodiment the invention concerns the above-mentioned compound of formula I, wherein L is absent and wherein A is selected from the group consisting of —CH₂— and —CF₂—, and prodrugs, pharmaceutical acceptable salts thereof or deuterated analogues thereof.

In a further preferred embodiment the invention relates to the above-mentioned compound of formula I, wherein L is absent and wherein K is CF₂, and prodrugs, deuterated analogues and pharmaceutical acceptable salts thereof.

In another preferred embodiment the invention concerns the above-mentioned compound of formula I, wherein R³ is halogen, and prodrugs, deuterated analogues and pharmaceutical acceptable salts thereof.

Hereby it is particularly preferred that R³ is a halogen atom selected from the group consisting of Cl and F.

In a further preferred embodiment the invention relates to the above-mentioned compound of formula I, wherein R³ is Cl or F and is located in the 5-position of the benzimidazole moiety, and prodrugs, deuterated analogues and pharmaceutical acceptable salts thereof.

In a further preferred embodiment the invention relates to the above-mentioned compound of formula I, wherein R¹ is halogen, and prodrugs, deuterated analogues and pharmaceutical acceptable salts thereof.

In another preferred embodiment the invention concerns the above-mentioned compound of formula I, wherein R¹ is Cl or F, and prodrugs, deuterated analogues and pharmaceutical acceptable salts thereof.

In a further preferred embodiment the invention relates to the above-mentioned compound of formula I, wherein R¹ is hydrogen, and prodrugs, deuterated analogues and pharmaceutical acceptable salts thereof.

In another particularly preferred embodiment the invention concerns the above-mentioned compound of formula I which is selected from the group consisting of

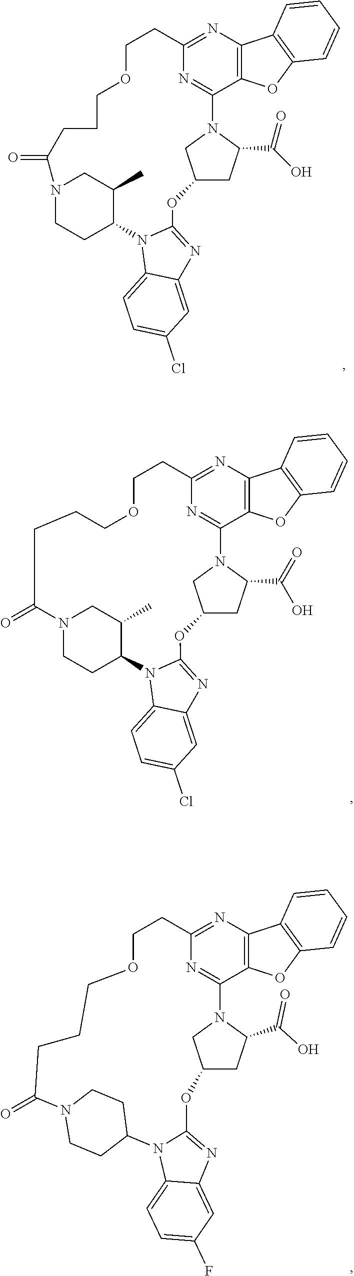 [SMILES: C[C@@H]1CN2CC[C@H]1n1c(nc3cc(Cl)ccc31)O[C@H]1C[C@@H](C(=O)O)N(C1)c1nc(nc3c1oc1ccccc13)CCOCCCC2=O ; C[C@H]1CN2CC[C@@H]1n1c(nc3cc(Cl)ccc31)O[C@H]1C[C@@H](C(=O)O)N(C1)c1nc(nc3c1oc1ccccc13)CCOCCCC2=O ; O=C(O)[C@@H]1C[C@H]2CN1c1nc(nc3c1oc1ccccc13)CCOCCCC(=O)N1CCC(CC1)n1c(nc3cc(F)ccc31)O2]

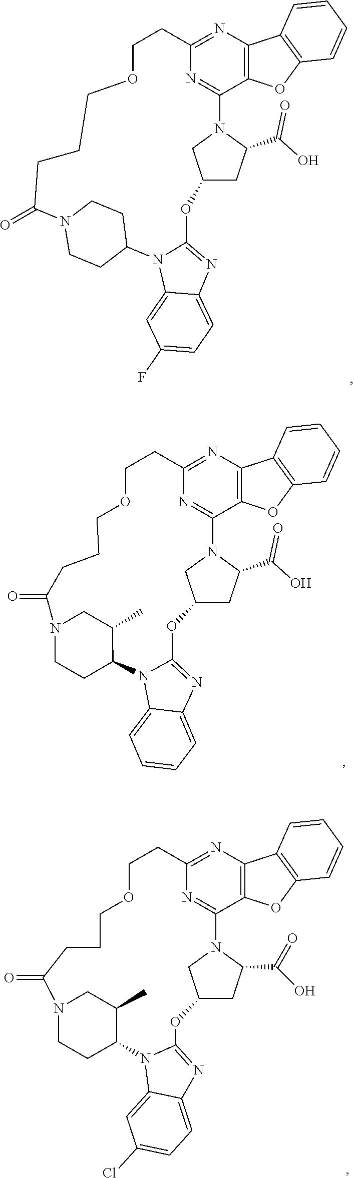 [SMILES: O=C(O)[C@@H]1C[C@H]2CN1c1nc(nc3c1oc1ccccc13)CCOCCCC(=O)N1CCC(CC1)n1c(nc3ccc(F)cc31)O2 ; C[C@H]1CN2CC[C@@H]1n1c(nc3ccccc31)O[C@H]1C[C@@H](C(=O)O)N(C1)c1nc(nc3c1oc1ccccc13)CCOCCCC2=O ; C[C@@H]1CN2CC[C@H]1n1c(nc3ccc(Cl)cc31)O[C@H]1C[C@@H](C(=O)O)N(C1)c1nc(nc3c1oc1ccccc13)CCOCCCC2=O]

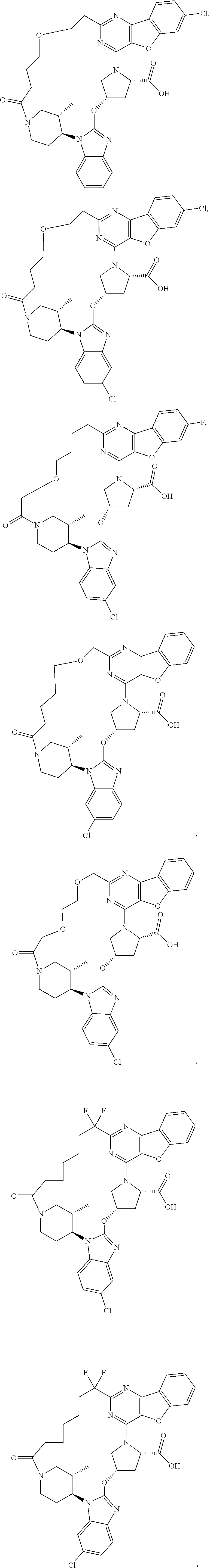

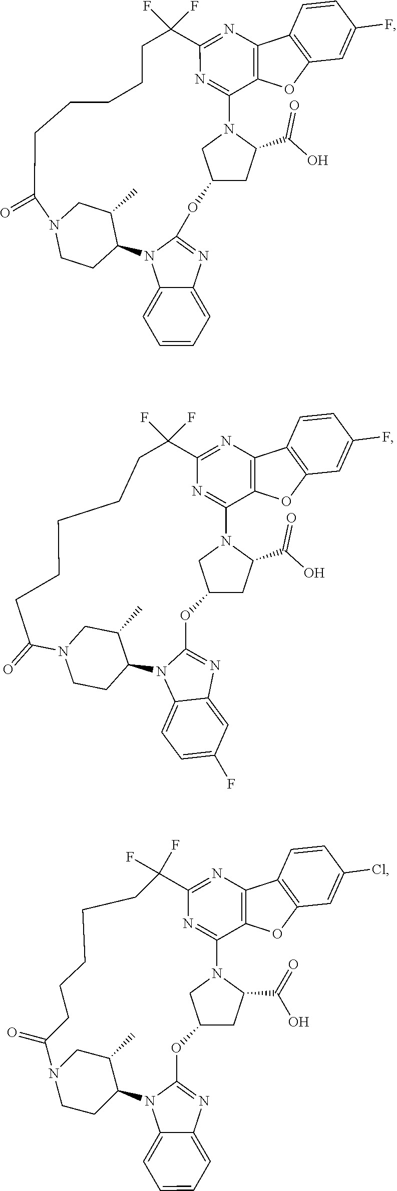 [SMILES: C[C@H]1CN2CC[C@@H]1n1c(nc3ccccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O]

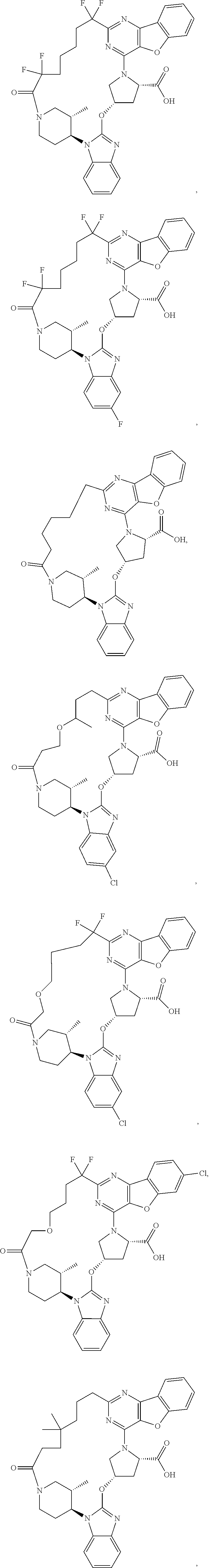

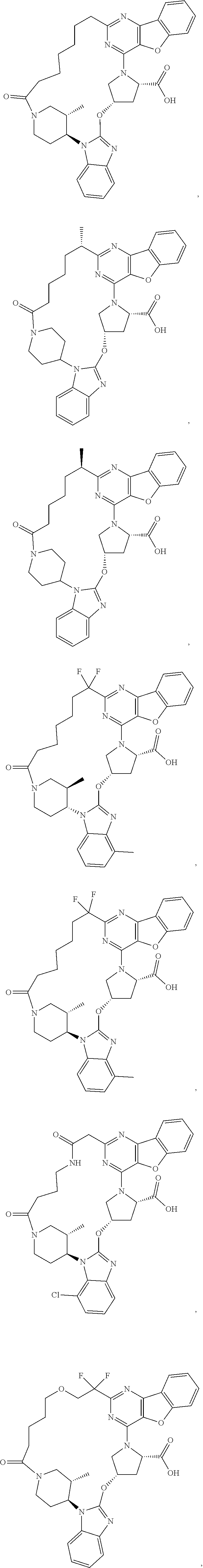

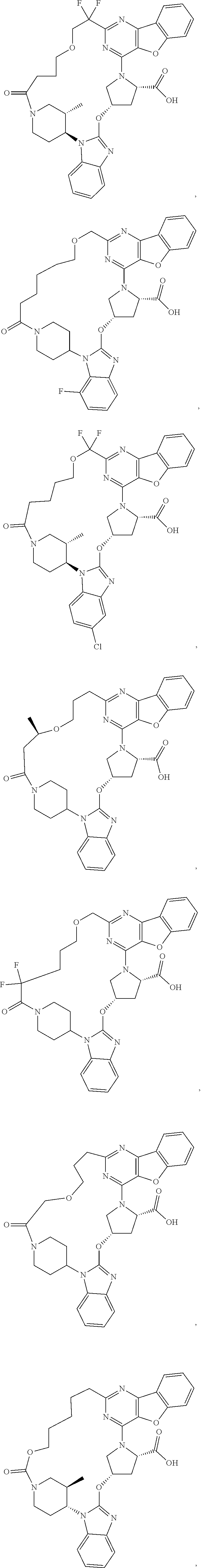

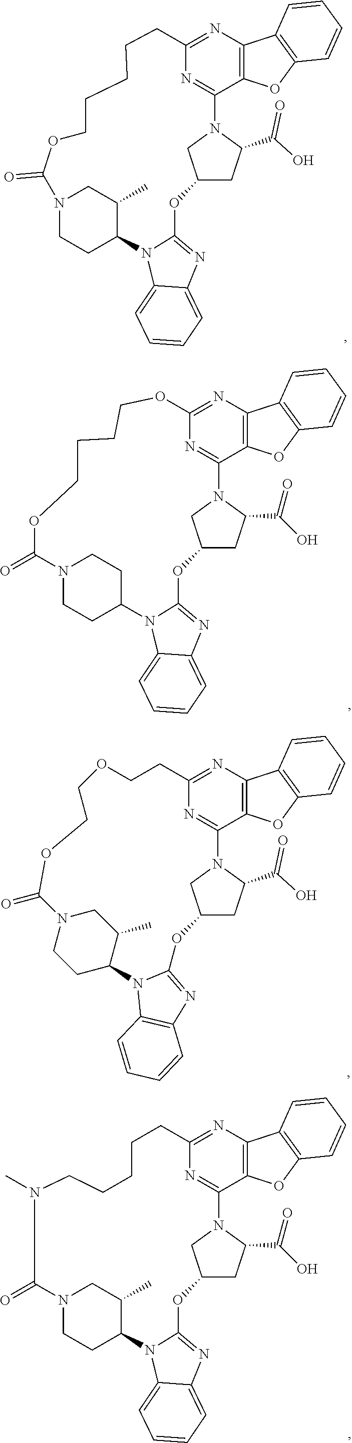

and prodrugs, deuterated analogues and pharmaceutical acceptable salts thereof.

In a further preferred embodiment the invention relates to the above-mentioned compound of formula I, wherein
A is selected from the group consisting of —CH₂— and —CF₂—,
D and E are each —CH₂—,
G is selected from the group consisting of —CH₂— and —O—,
J is selected from the group consisting of —CH₂— and —O—,
K is selected from the group consisting of —CF₂— and —CH₂—,
and L is absent,
and prodrugs, deuterated analogues and pharmaceutical acceptable salts thereof.

In another particularly preferred embodiment the invention concerns the above-mentioned compound of formula I which is selected from the group consisting of

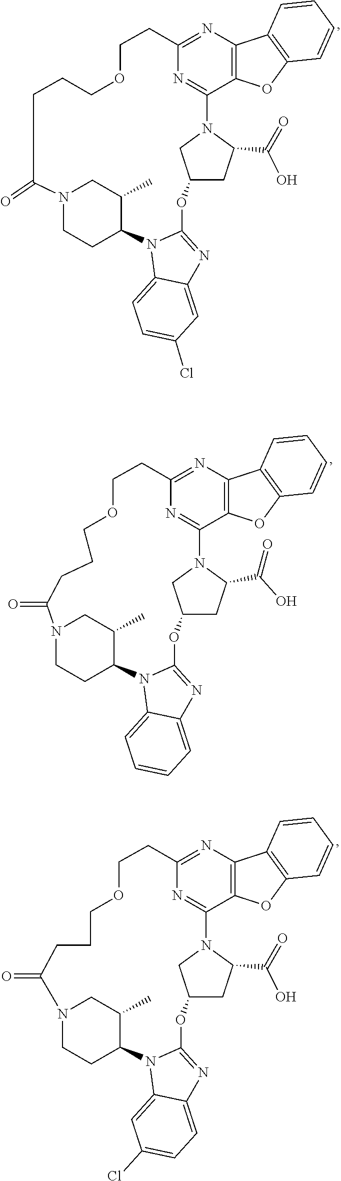 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)CCOCCCC2=O ; C[C@H]1CN2CC[C@@H]1n1c(nc3ccccc31)O[C@H]1C[C@@H](C(=O)O)N(C1)c1nc(nc3c1oc1ccccc13)CCOCCCC2=O ; C[C@H]1CN2CC[C@@H]1n1c(nc3ccc(Cl)cc31)O[C@H]1C[C@@H](C(=O)O)N(C1)c1nc(nc3c1oc1ccccc13)CCOCCCC2=O]

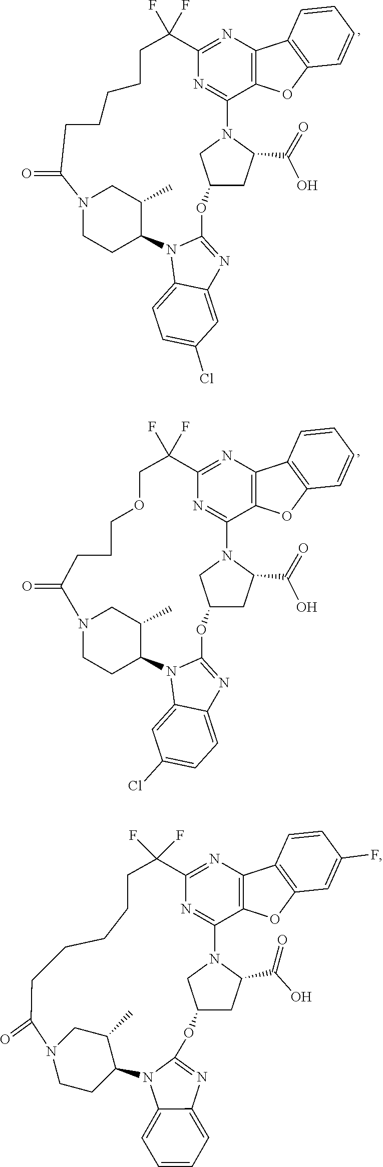

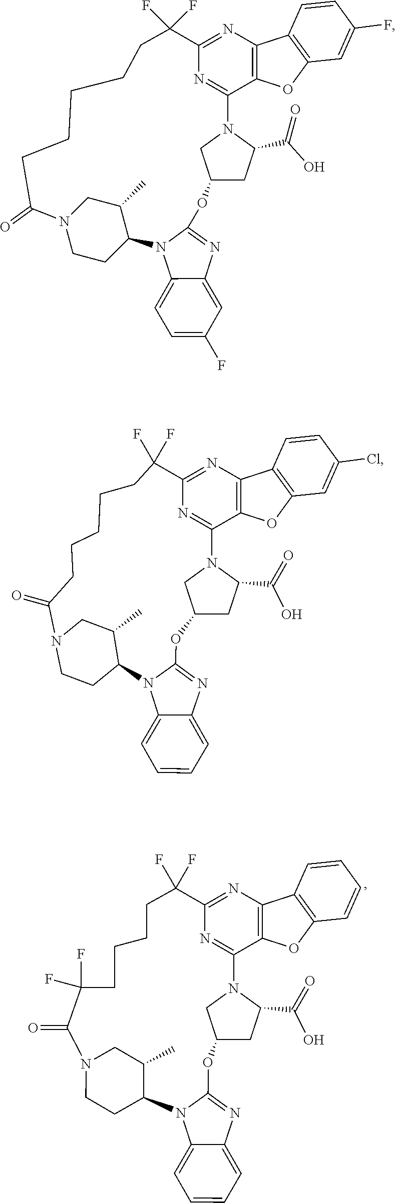 [SMILES: C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1ccccc13)C(F)(F)CCCCC(F)(F)C2=O]

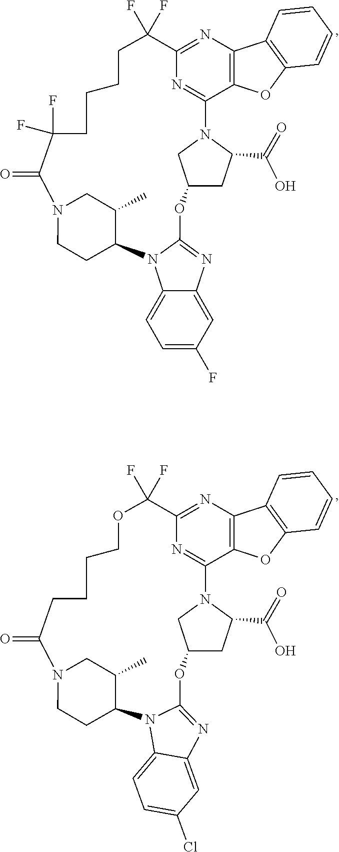 [SMILES: C[C@H]1CN2CC[C@@H]1n1c(nc3cc(F)ccc31)O[C@H]1C[C@@H](C(=O)O)N(C1)c1nc(nc3c1oc1ccccc13)C(F)(F)CCCCC(F)(F)C2=O ; C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O]

and prodrugs, deuterated analogues and pharmaceutical acceptable salts thereof.

In a further preferred embodiment the invention relates to the above-mentioned compound of formula I, wherein R₂ is methyl, and prodrugs, deuterated analogues and pharmaceutical acceptable salts thereof.

In another particularly preferred embodiment the invention concerns the compound of formula II

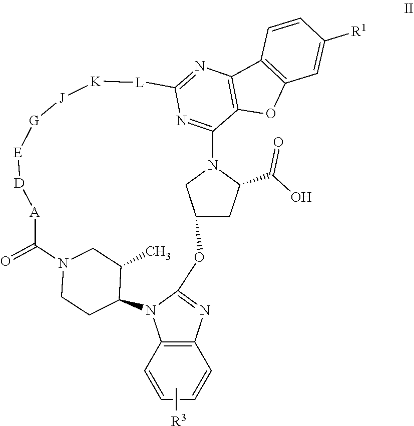

or the compound of formula III

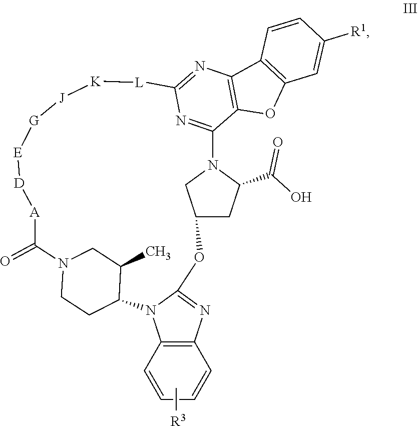

wherein
R¹ is selected from the group consisting of hydrogen and halogen,
R³ is selected from the group consisting of hydrogen, methyl and halogen,
and wherein
A is selected from the group consisting of —CH₂—, —O—, —CF₂—, —CHF—, —N(CH₃)—, —NH— and —CHCH₃—;
D is selected from the group consisting of —CH₂—, —O—, —CF₂—, —CHF— and —CHCH₃—;
E is selected from the group consisting of —CH₂—, —O—, —C(CH₃)₂—, —CHF—, CF₂— and —CHCH₃—;
G is selected from the group consisting of —NH—, —NCH₃—, —CH₂—, —O—, —CF₂—, —CHF—, —CHCH₃— and —C(CH₃)₂;
J is selected from the group consisting of —CO—, —CH₂—, —O—, —CHF—, —CF₂— and —CHCH₃—;
K is selected from the group consisting of —CH₂—, —CF₂—, —O— or is absent;
L is selected from the group consisting of —CH₂—, —O—, —CHCH₃—, —CHF—, —CF₂— or is absent;
and prodrugs, deuterated analogues and pharmaceutical acceptable salts thereof.

Hereby variables A, D, E, G, J, K and L are preferably selected in such a way that two or more heteroatoms may not follow directly upon each other.

Further preferred is the above-mentioned compound of formula II or of formula III,

wherein
R¹ is selected from the group consisting of hydrogen, Cl and F,
R³ is selected from the group consisting of hydrogen, methyl, Cl and F,
and wherein
A is selected from the group consisting of —CH₂—, —O—, —CF₂— and —N(CH₃)—,
D is selected from the group consisting of —CH₂—, —O— and —CHCH₃—,
E is selected from the group consisting of —CH₂—, —O— and —C(CH₃)₂—,
G is selected from the group consisting of —NH—, —CH₂—, —O—, —CHCH₃—,
J is selected from the group consisting of —CO—, —CH₂—, —O—, and —CHCH₃—;
K is selected from the group consisting of —CH₂—, —CF₂—, —O— or is absent;
L is selected from the group consisting of —CH₂—, —CF₂— or is absent;
and prodrugs, deuterated analogues and pharmaceutical acceptable salts thereof.

Hereby variables A, D, E, G, J, K and L are preferably selected in such a way that two or more heteroatoms may not follow directly upon each other.

Particularly preferred is the above-mentioned compound of formula II,

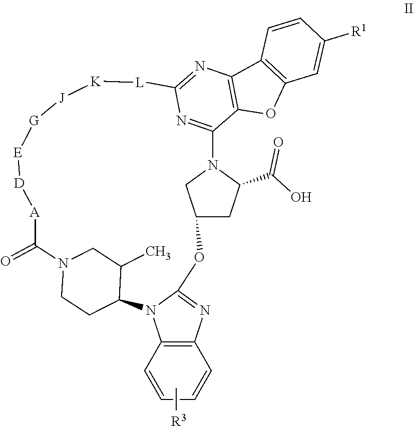

wherein
R¹ is selected from the group consisting of hydrogen, Cl and F,
R³ is selected from the group consisting of hydrogen, methyl, Cl and F,
and wherein
A is selected from the group consisting of —CH₂—, —O—, —CF₂— and —N(CH₃)—,
D is selected from the group consisting of —CH₂—, —O— and —CHCH₃—,
E is selected from the group consisting of —CH₂—, —O— and —C(CH₃)₂—,
G is selected from the group consisting of —NH—, —CH₂—, —O—, —CHCH₃—,
J is selected from the group consisting of —CO—, —CH₂—, —O—, and —CHCH₃—;
K is selected from the group consisting of —CH₂—, —CF₂—, —O— or is absent;
L is selected from the group consisting of —CH₂—, —CF₂— or is absent;
and prodrugs, deuterated analogues and pharmaceutical acceptable salts thereof.

Hereby variables A, D, E, G, J, K and L are preferably selected in such a way that two or more heteroatoms may not follow directly upon each other.

In another preferred embodiment the invention concerns the above-mentioned compound of formula II or the above-mentioned compound of formula III, wherein L is absent, and prodrugs, deuterated analogues and pharmaceutical acceptable salts thereof.

In a further preferred embodiment the invention relates to the above-mentioned compound of formula II or the above-mentioned compound of formula III, wherein L is absent and wherein K is CF₂, and prodrugs, deuterated analogues and pharmaceutical acceptable salts thereof.

In another preferred embodiment the invention concerns the above-mentioned compound of formula II or the above-mentioned compound of formula III, wherein R³ is halogen, and prodrugs, deuterated analogues and pharmaceutical acceptable salts thereof.

In a further preferred embodiment the invention relates to the above-mentioned compound of formula II or to the above-mentioned compound of formula III, wherein R³ is Cl or F, and prodrugs, deuterated analogues and pharmaceutical acceptable salts thereof.

In a particularly preferred embodiment the invention relates to the above-mentioned compound of formula II or to the above-mentioned compound of formula III, wherein R³ is Cl either or F and is located in the 5-position of the benzimidazole moiety, and prodrugs, deuterated analogues and pharmaceutical acceptable salts thereof.

In another preferred embodiment the invention concerns the above-mentioned compound of formula II or the above-mentioned compound of formula III, wherein R³ is hydrogen, and prodrugs, deuterated analogues and pharmaceutical acceptable salts thereof.

In a further preferred embodiment the invention relates to the above-mentioned compound of formula II or to the above-mentioned compound of formula III, wherein R¹ is halogen, and prodrugs, deuterated analogues and pharmaceutical acceptable salts thereof.

In particularly preferred embodiment the invention concerns the above-mentioned compound of formula II or the above-mentioned compound of formula III, wherein R¹ is selected from the group consisting of Cl or F, and prodrugs, deuterated analogues and pharmaceutical acceptable salts thereof.

In another preferred embodiment the invention concerns the above-mentioned compound of formula II or the above-mentioned compound of formula III, wherein R¹ is hydrogen, and prodrugs, deuterated analogues and pharmaceutical acceptable salts thereof.

In a further preferred embodiment the invention relates to the above-mentioned compound of formula II or to the above-mentioned compound of formula III, wherein
A is selected from the group consisting of —CH₂— and —CF₂—,
D and E are each —CH₂—,
G is selected from the group consisting of —CH₂— and —O—,
J is selected from the group consisting of —CH₂— and —O—,
K is selected from the group consisting of —CF₂— and —CH₂—,
and L is absent,
and prodrugs, deuterated analogues and pharmaceutical acceptable salts thereof.

In another particularly preferred embodiment the invention concerns the above-mentioned compound of formula II, which is selected from the group consisting of

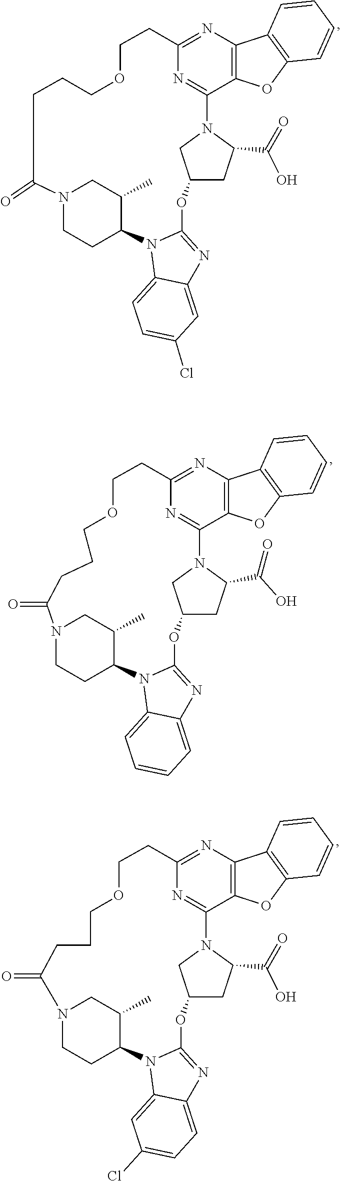 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)CCOCCCC2=O ; C[C@H]1CN2CC[C@@H]1n1c(nc3ccccc31)O[C@H]1C[C@@H](C(=O)O)N(C1)c1nc(nc3c1oc1ccccc13)CCOCCCC2=O ; C[C@H]1CN2CC[C@@H]1n1c(nc3ccc(Cl)cc31)O[C@H]1C[C@@H](C(=O)O)N(C1)c1nc(nc3c1oc1ccccc13)CCOCCCC2=O]

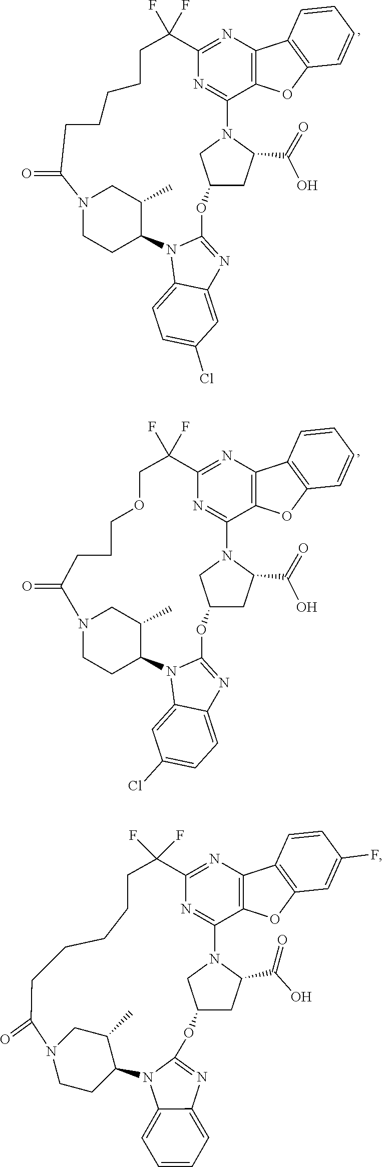

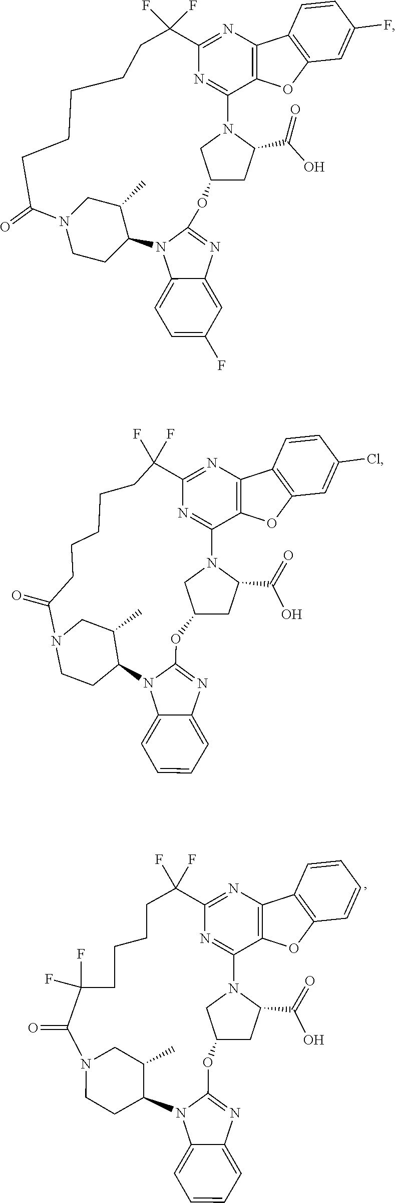 [SMILES: C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1ccccc13)C(F)(F)CCCCC(F)(F)C2=O]

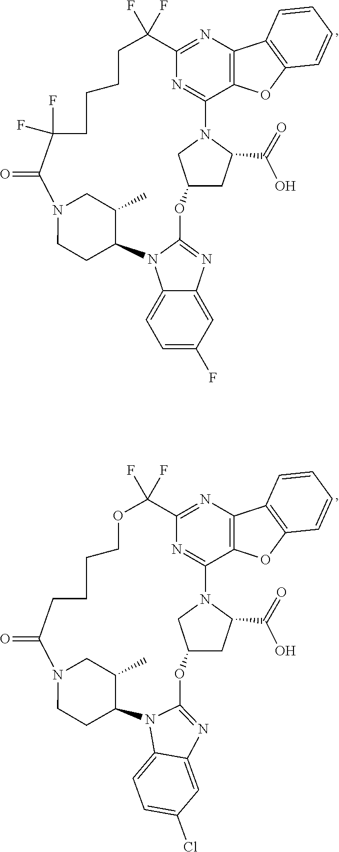 [SMILES: C[C@H]1CN2CC[C@@H]1n1c(nc3cc(F)ccc31)O[C@H]1C[C@@H](C(=O)O)N(C1)c1nc(nc3c1oc1ccccc13)C(F)(F)CCCCC(F)(F)C2=O ; C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O]

and prodrugs, deuterated analogues and pharmaceutical acceptable salts thereof.

In a further particularly preferred embodiment the invention concerns the above-mentioned compound of formula II or the above-mentioned compound of formula III, wherein
A is selected from the group consisting of —CH₂— and —CF₂—,
D, E and G are each —CH₂—,
J is selected from the group consisting of —CH₂— and —O—,
K is —CF₂—,
and L is absent,
and prodrugs, deuterated analogues and pharmaceutical acceptable salts thereof.

In another particularly preferred embodiment the invention concerns the above-mentioned compound of formula II, which is selected from the group consisting of

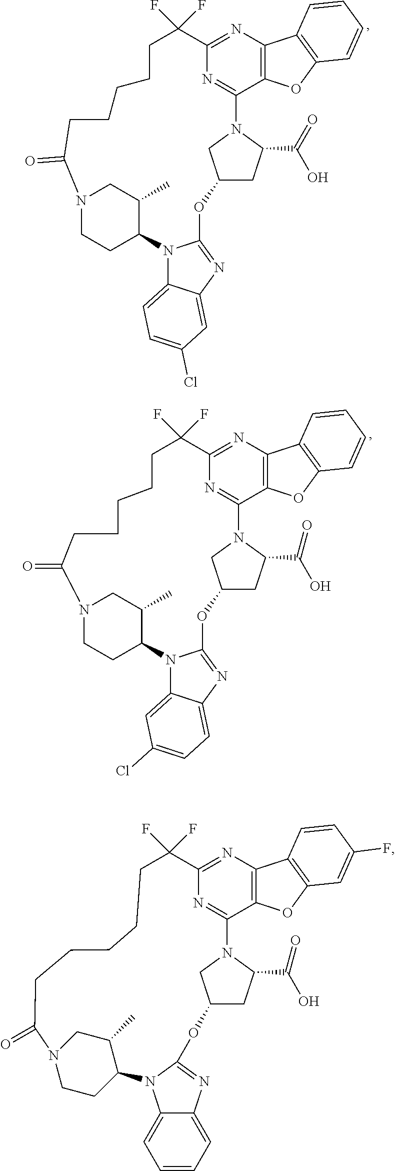

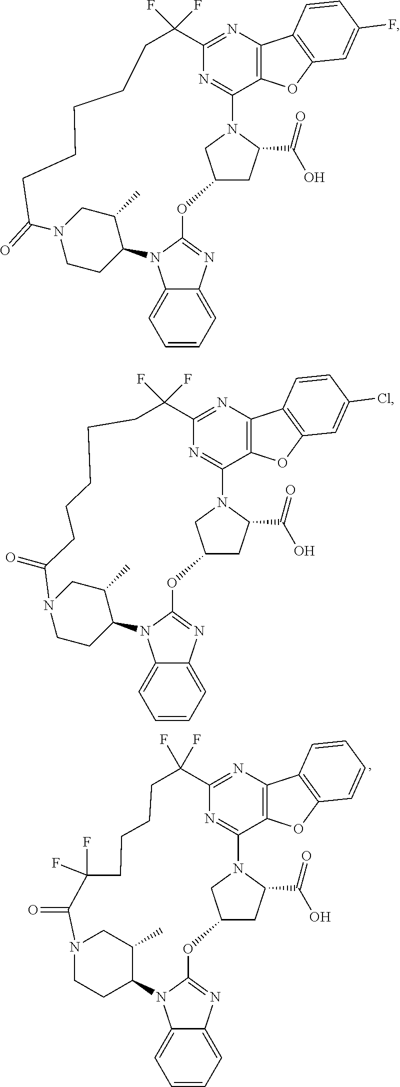

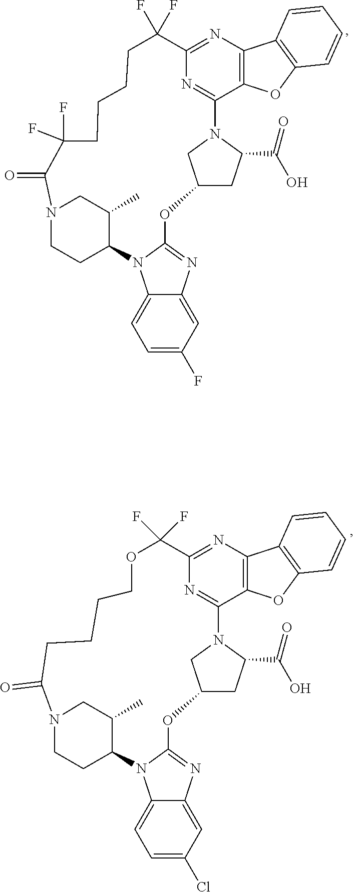 [SMILES: C[C@H]1CN2CC[C@@H]1n1c(nc3cc(F)ccc31)O[C@H]1C[C@@H](C(=O)O)N(C1)c1nc(nc3c1oc1ccccc13)C(F)(F)CCCCC(F)(F)C2=O ; C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O]

and prodrugs, deuterated analogues and pharmaceutical acceptable salts thereof.

Prodrugs of the compounds of formula I are preferably compounds of formula Ia

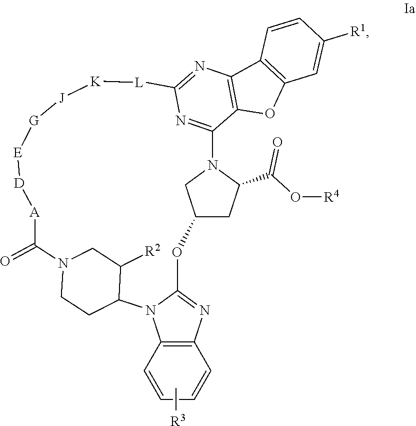

wherein variables R¹, R², R³, A, D, E, G, J, K and L are defined as aforementioned and
wherein R⁴ is C₁₋₄-alkyl, aryl, —CH₂-aryl, NH—SO₂—C₁₋₃-alkyl.

Particularly preferred are the prodrugs of formula Ia, wherein variables R¹, R², R³, A, D, E, G, J, K and L are defined as aforementioned and wherein R⁴ is methyl.

Prodrugs of the compounds of formula II are preferably compounds of formula IIa

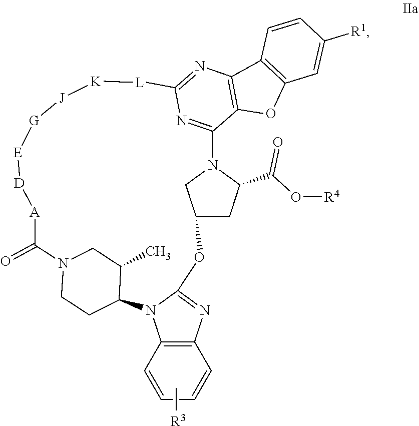

wherein variables R¹, R², R³, A, D, E, G, J, K and L are defined as aforementioned and
wherein R⁴ is C₁₋₄-alkyl, aryl, —CH₂-aryl, NH—SO₂—C₁₋₃-alkyl.

Particularly preferred are the prodrugs of formula IIa, wherein variables R¹, R², R³, A, D, E, G, J, K and L are defined as aforementioned and wherein R⁴ is methyl.

Prodrugs of the compounds of formula III are preferably compounds of formula IIIa

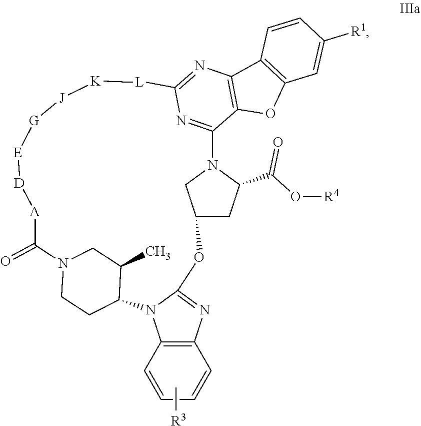

wherein variables R¹, R², R³, A, D, E, G, J, K and L are defined as aforementioned and
wherein R⁴ is C₁₋₄-alkyl, aryl, —CH₂-aryl, NH—SO₂—C₁₋₃-alkyl.

Particularly preferred are the prodrugs of formula IIIa, wherein variables R¹, R², R³, A, D, E, G, J, K and L are defined as aforementioned and wherein R⁴ is methyl.

In another preferred embodiment the invention relates to
a) intermediate compounds of formula (A-I)

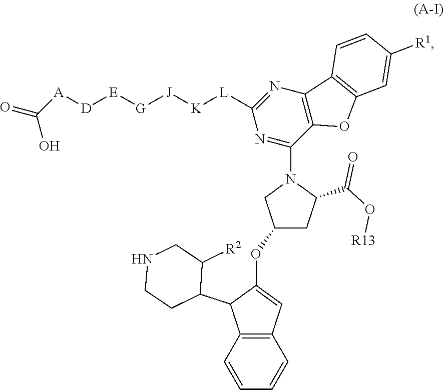

wherein R¹, R², R³, A, D, E, G, J, K and L are defined as above-mentioned and wherein R13 is selected from the group consisting of hydrogen, methyl, ethyl and tert-butyl,
b) intermediate compounds of formula (A-II),

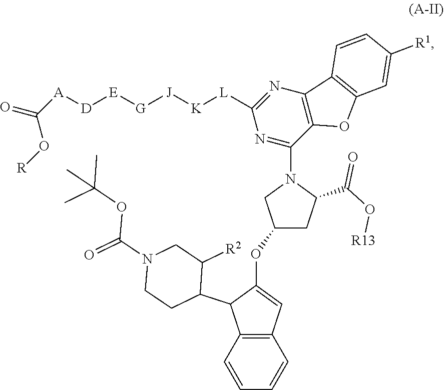

wherein R¹, R², R³, A, D, E, G, J, K and L are defined as above-mentioned and wherein R13 is selected from the group consisting of hydrogen, methyl, ethyl and tert-butyl,
and wherein R is either hydrogen or a protecting group selected from the group consisting of tert-butyl, methyl, ethyl and benzyl,
c) intermediate compounds of formula (B-1)

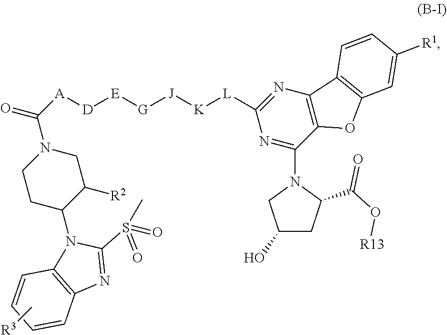

wherein R¹, R², R³, A, D, E, G, J, K and L are defined as above-mentioned and wherein R13 is selected from the group consisting of hydrogen, methyl, ethyl and tert-butyl,
or
d) intermediate compounds of formula (B-II)

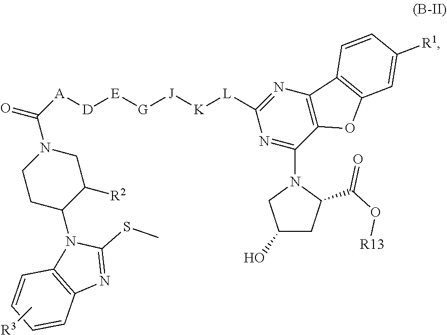

wherein R¹, R², R³, A, D, E, G, J, K and L are defined as above-mentioned and wherein R13 is selected from the group consisting of hydrogen, methyl, ethyl and tert-butyl.

In a further preferred embodiment the invention concerns an above-mentioned compound of formula I, II or III or a prodrug of formula Ia, IIa or IIIa, deuterated analogues and pharmaceutical acceptable salts thereof, for use in the treatment of a disease that can be treated by the inhibition of cGAS.

In a further preferred embodiment the invention relates to an above-mentioned compound of formula I, II or III or a prodrug of formula Ia, IIa or IIIa, deuterated analogues and pharmaceutical acceptable salts thereof, for use in the treatment of a disease selected from the group consisting of systemic lupus erythematosus (SLE), interferonopathies, Aicardi-Goutières syndrome (AGS), COPA syndrome, familial chilblain lupus, age-related macular degeneration (AMD), retinopathy, glaucoma, amyotrophic lateral sclerosis (ALS), diabetes, obesity, inflammatory bowel disease (IBD), chronic obstructive pulmonary disease (COPD), Bloom's syndrome, Sjogren's syndrome, Parkinsons disease, heart failure, cancer, systemic sclerosis (SSc), dermatomyositis, non-alcoholic steatotic hepatitis (NASH), interstitial lung disease (ILD), preferably progressive fibrosing interstitial lung disease (PF-ILD), in particular idiopathic pulmonary fibrosis (IPF), aging, muscle disorders, sepsis, rheumatoid arthritis, osteoarthritis and COVID-19.

In another preferred embodiment the invention relates to an above-mentioned compound of formula I, II or III or a prodrug of formula Ia, IIa or IIIa, deuterated analogues and pharmaceutical acceptable salts thereof, for use in the treatment of a disease selected from the group consisting of systemic lupus erythematosus (SLE), interferonopathies, Aicardi-Goutières syndrome (AGS), COPA syndrome, familial chilblain lupus, dermatomyositis, age-related macular degeneration (AMD), amyotrophic lateral sclerosis (ALS), inflammatory bowel disease (IBD), chronic obstructive pulmonary disease (COPD), Bloom's syndrome, Sjogren's syndrome, rheumatoid arthritis and Parkinsons disease.

In a further preferred embodiment the invention relates to an above-mentioned compound of formula I, II or III or a prodrug of formula Ia, IIa or IIIa, deuterated analogues and pharmaceutical acceptable salts thereof, for use in the treatment of a disease selected from the group consisting of systemic sclerosis (SSc), non-alcoholic steatohepatitis (NASH), interferonopathies, interstitial lung disease (ILD), preferably progressive fibrosing interstitial lung disease (PF-ILD), in particular idiopathic pulmonary fibrosis (IPF).

In another preferred embodiment the invention relates to an above-mentioned compound of formula I, II or III or a prodrug of formula Ia, IIa or IIIa, deuterated analogues and pharmaceutical acceptable salts thereof, for use in the treatment of a disease selected from the group consisting of age-related macular degeneration (AMD), retinopathy, glaucoma, diabetes, obesity, aging, muscle disorders, sepsis, osteoarthritis, heart failure, COVID-19/SARS-CoV-2 infection, renal inflammation, renal fibrosis, dysmetabolism, vascular diseases, cardiovascular diseases and cancer.

In another preferred embodiment the invention relates to a pharmaceutical composition comprising an above-mentioned compound of formula I, II or III or a prodrug of formula Ia, IIa or IIIa, deuterated analogues and pharmaceutical acceptable salts thereof, and optionally one or more pharmaceutically acceptable carriers and/or excipients.

In another preferred embodiment the invention relates to a pharmaceutical composition comprising an above-mentioned compound of formula I, II or III or a prodrug of formula Ia, IIa or IIIa, deuterated analogues and pharmaceutical acceptable salts thereof, in combination with one or more active agents selected from the group consisting of anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics such as interferons or other cytokines/chemokines, cytokine/chemokine receptor modulators, toll-like receptor agonists, immune checkpoint regulators, an anti-TNF antibody such as Humira™, an anti-BAFF antibody such as Belimumab and Etanercept, and optionally one or more pharmaceutically acceptable carriers and/or excipients.

In a further preferred embodiment the invention relates to a pharmaceutical composition comprising an above-mentioned compound of formula I, II or III or a prodrug of formula Ia, IIa or IIIa, deuterated analogues and pharmaceutical acceptable salts thereof, and one or more anti-fibrotic agents selected from the group consisting of Pirfenidon and Nintedanib and optionally one or more pharmaceutically acceptable carriers and/or excipients.

In another preferred embodiment the invention relates to a pharmaceutical composition comprising an above-mentioned compound of formula I, II or III or a prodrug of formula Ia, IIa or IIIa, deuterated analogues and pharmaceutical acceptable salts thereof, and one or more anti-inflammatory agents selected from the group consisting of NSAIDs and corticosteroids and optionally one or more pharmaceutically acceptable carriers and/or excipients.

In a further preferred embodiment the invention relates to a pharmaceutical composition comprising an above-mentioned compound of formula I, II or III or a prodrug of formula Ia, IIa or IIIa, deuterated analogues and pharmaceutical acceptable salts thereof, and one or more active agents selected from the group consisting of bronchodilators, beta 2 agonists/betamimetics, adrenergic agonists and anticholinergic agents and optionally one or more pharmaceutically acceptable carriers and/or excipients.

In another preferred embodiment the invention relates to a pharmaceutical combination comprising an above-mentioned compound of formula I, II or III or a prodrug of formula Ia, IIa or IIIa, deuterated analogues and pharmaceutical acceptable salts thereof, and one or more anti-interleukin antibodies selected from the group consisting of anti-IL-23 such as Risankizumab, anti-IL-17 antibodies, anti-IL-1 antibodies, anti-IL-4 antibodies, anti-IL-13 antibodies, anti-IL-5 antibodies, anti-IL-6 antibodies such as Actemra™, anti-IL-12 antibodies and anti-IL-15 antibodies.

### 3. Terms and Definitions Used

Unless stated otherwise, all the substituents are independent of one another. If for example a number of C₁₋₆-alkyl groups are possible substituents at a group, in the case of three substituents, for example, C₁₋₆-alkyl could represent, independently of one another, a methyl, a n-propyl and a tert-butyl.

A crossed bond like the middle bond in following butyl-molecule

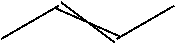

represents a double bond of unknown configuration (either cis, trans or a mixture thereof).

By the term “C₁₋₆-alkyl” (including those which are part of other groups) are meant branched and unbranched alkyl groups with 1 to 6 carbon atoms and by the term “C₁₋₃-alkyl” are meant branched and unbranched alkyl groups with 1 to 3 carbon atoms. “C₁₋₄-alkyl” accordingly denotes branched and unbranched alkyl groups with 1 to 4 carbon atoms. Alkyl groups with 1 to 4 carbon atoms are preferred. Examples of these include methyl, ethyl, n-propyl, iso-propyl, n-butyl, iso-butyl, sec-butyl, tert-butyl, n-pentyl, iso-pentyl, neo-pentyl and hexyl. The abbreviations Me, Et, n-Pr, i-Pr, n-Bu, i-Bu, t-Bu, etc., may also optionally be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₁₋₆-alkylene” (including those which are part of other groups) are meant branched and unbranched alkylene groups with 1 to 6 carbon atoms and by the term “C₁₋₄-alkylene” are meant branched and unbranched alkylene groups with 1 to 4 carbon atoms. Alkylene groups with 1 to 4 carbon atoms are preferred. Examples of these include methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene and hexylene. Unless stated otherwise, the definitions propylene, butylene, pentylene and hexylene include all the possible isomeric forms of the groups in question with the same number of carbons. Thus, for example, propyl includes also 1-methylethylene and butylene includes 1-methylpropylene, 1,1-dimethylethylene, 1,2-dimethylethylene etc.

If the carbon chain is substituted by a group which together with one or two carbon atoms of the alkylene chain forms a carbocyclic ring with 3, 5 or 6 carbon atoms, this includes, inter alia, the following examples of the rings:

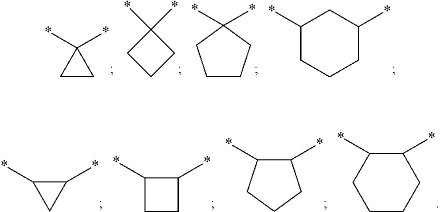

By the term “C₂₋₆-alkenyl” (including those which are part of other groups) are meant branched and unbranched alkenyl groups with 2 to 6 carbon atoms and by the term “C₂₋₄-alkenyl” are meant branched and unbranched alkenyl groups with 2 to 4 carbon atoms, provided that they have at least one double bond. Alkenyl groups with 2 to 4 carbon atoms are preferred. Examples include: ethenyl or vinyl, propenyl, butenyl, pentenyl or hexenyl. Unless stated otherwise, the definitions propenyl, butenyl, pentenyl and hexenyl include all the possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

By the term “C₂-s-alkynyl” (including those which are part of other groups) are meant branched and unbranched alkynyl groups with 2 to 5 carbon atoms and by the term “C₂₋₄-alkynyl” are meant branched and unbranched alkynyl groups with 2 to 4 carbon atoms, provided that they have at least one triple bond. Alkynyl groups with 2 to 4 carbon atoms are preferred.

By the term “C₂₋₆-alkenylene” (including those which are part of other groups) are meant branched and unbranched alkenylene groups with 2 to 6 carbon atoms and by the term “C₂₋₄-alkenylene” are meant branched and unbranched alkylene groups with 2 to 4 carbon atoms. Alkenylene groups with 2 to 4 carbon atoms are preferred. Examples of these include: ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene and hexenylene. Unless stated otherwise, the definitions propenylene, butenylene, pentenylene and hexenylene include all the possible isomeric forms of the groups in question with the same number of carbons. Thus, for example, propenyl also includes 1-methylethenylene and butenylene includes 1-methylpropenylene, 1,1-dimethylethenylene, 1, 2-dimethylethenylene.

By the term “aryl” (including those which are part of other groups) are meant aromatic ring systems with 6 or 10 carbon atoms. Examples include phenyl or naphthyl, the preferred aryl group being phenyl. Unless otherwise stated, the aromatic groups may be substituted by one or more groups selected from among methyl, ethyl, iso-propyl, tert-butyl, hydroxy, fluorine, chlorine, bromine and iodine.

By the term “aryl-C₁₋₆-alkylene” (including those which are part of other groups) are meant branched and unbranched alkylene groups with 1 to 6 carbon atoms, which are substituted by an aromatic ring system with 6 or 10 carbon atoms. Examples include benzyl, 1- or 2-phenylethyl and 1- or 2-naphthylethyl. Unless otherwise stated, the aromatic groups may be substituted by one or more groups selected from among methyl, ethyl, iso-propyl, tert-butyl, hydroxy, fluorine, chlorine, bromine and iodine.

By the term “heteroaryl-C₁₋₆-alkylene” (including those which are part of other groups) are meant—even though they are already included under “aryl-C₁₋₆-alkylene”-branched and unbranched alkylene groups with 1 to 6 carbon atoms, which are substituted by a heteroaryl.

If not specifically defined otherwise, a heteroaryl of this kind includes five- or six-membered heterocyclic aromatic groups or 5-10-membered, bicyclic heteroaryl rings which may contain one, two, three or four heteroatoms selected from among oxygen, sulfur and nitrogen, and contain so many conjugated double bonds that an aromatic system is formed. The following are examples of five- or six-membered heterocyclic aromatic groups and bicyclic heteroaryl rings:

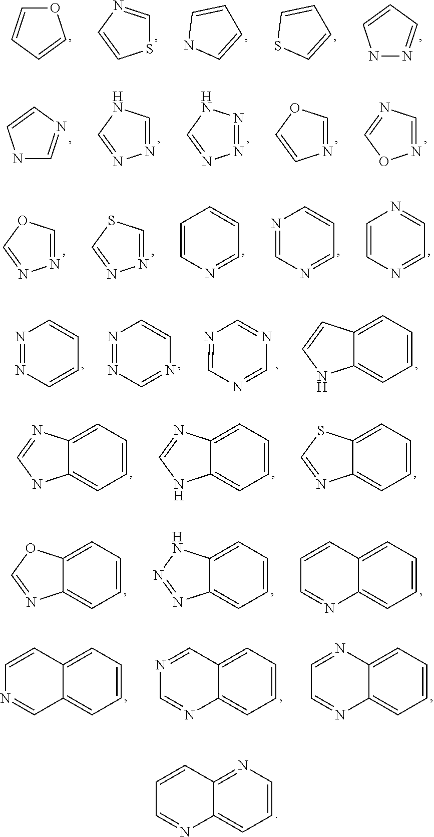

Unless otherwise stated, these heteroaryls may be substituted by one or more groups selected from among methyl, ethyl, iso-propyl, tert-butyl, hydroxy, amino, nitro, alkoxy, fluorine, chlorine, bromine and iodine.

The following are examples of heteroaryl-C₁₋₆-alkylenes:

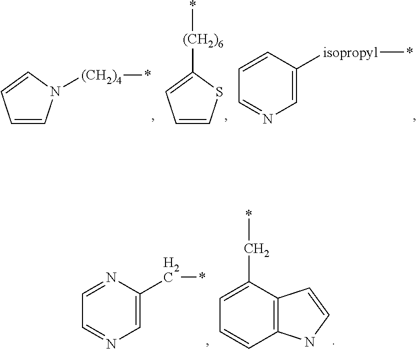

By the term “C₁₋₆-haloalkyl” (including those which are part of other groups) are meant branched and unbranched alkyl groups with 1 to 6 carbon atoms, which are substituted by one or more halogen atoms. By the term “C₁₋₄-haloalkyl” are meant branched and unbranched alkyl groups with 1 to 4 carbon atoms, which are substituted by one or more halogen atoms. Alkyl groups with 1 to 4 carbon atoms are preferred. Examples include: CF₃, CHF₂, CH₂F, CH₂CF₃.

By the term “C₃₋₇-cycloalkyl” (including those which are part of other groups) are meant cyclic alkyl groups with 3 to 7 carbon atoms, if not specifically defined otherwise. Examples include: cyclopropyl, cyclobutyl, cyclopentyl, cyclohexyl and cycloheptyl. Unless otherwise stated, the cyclic alkyl groups may be substituted by one or more groups selected from among methyl, ethyl, iso-propyl, tert-butyl, hydroxy, fluorine, chlorine, bromine and iodine.

If not specifically defined otherwise, by the term “C₃₋₁₀-cycloalkyl” are also meant monocyclic alkyl groups with 3 to 7 carbon atoms and also bicyclic alkyl groups with 7 to 10 carbon atoms, or monocyclic alkyl groups which are bridged by at least one C₁₋₃-carbon bridge.

By the term “heterocyclic rings” or “heterocycle” are meant, unless stated otherwise, five-, six- or seven-membered, saturated, partially saturated or unsaturated heterocyclic rings which may contain one, two or three heteroatoms selected from among oxygen, sulfur and nitrogen, while the ring may be linked to the molecule through a carbon atom or through a nitrogen atom, if there is one. Although included by the term “heterocyclic rings” or “heterocycles”, the term “saturated heterocyclic ring” refers to five-, six- or seven-membered saturated rings. Examples include:

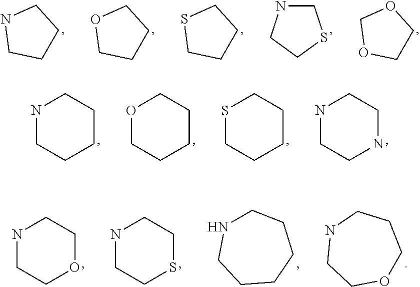

Although included by the term “heterocyclic rings” or “heterocyclic group”, the term “partially saturated heterocyclic group” refers to five-, six- or seven-membered partially saturated rings which contain one or two double bonds, without so many double bonds being produced that an aromatic system is formed, unless specifically defined otherwise. Examples include:

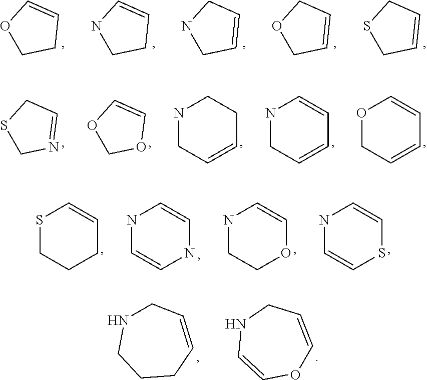

Although included by the term “heterocyclic rings” or “heterocycles”, the term “heterocyclic aromatic rings”, “unsaturated heterocyclic group” or “heteroaryl” refers to five- or six-membered heterocyclic aromatic groups or 5-10-membered, bicyclic heteroaryl rings which may contain one, two, three or four heteroatoms selected from among oxygen, sulfur and nitrogen, and contain so many conjugated double bonds that an aromatic system is formed, unless not specifically defined otherwise. Examples of five- or six-membered heterocyclic aromatic groups include:

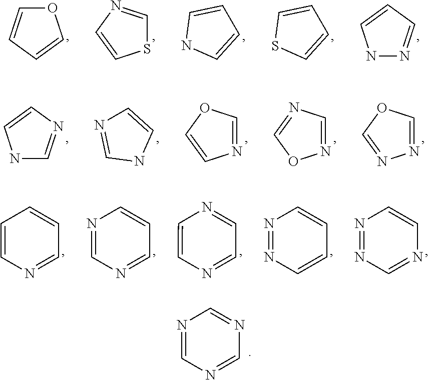

Unless otherwise mentioned, a heterocyclic ring (or heterocycle) may be provided with a keto group.

Examples include:

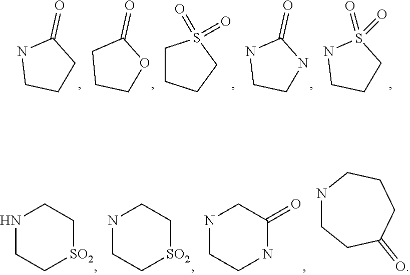

Although covered by the term “cycloalkyl”, the term “bicyclic cycloalkyls” generally denotes eight-, nine- or ten-membered bicyclic carbon rings. Examples include:

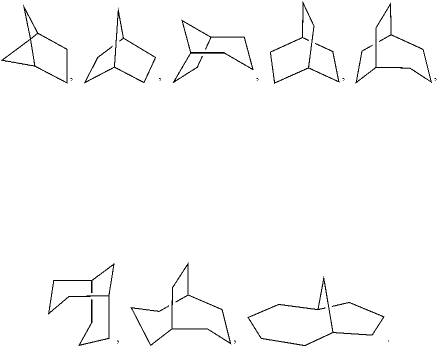

Although already included by the term “heterocycle”, the term “bicyclic heterocycles” generally denotes eight-, nine- or ten-membered bicyclic rings which may contain one or more heteroatoms, preferably 1-4, more preferably 1-3, even more preferably 1-2, particularly one heteroatom, selected from among oxygen, sulfur and nitrogen, unless not specifically defined otherwise. The ring may be linked to the molecule through a carbon atom of the ring or through a nitrogen atom of the ring, if there is one. Examples include:

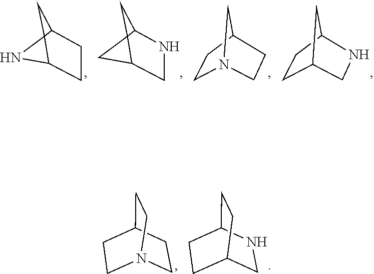

Although already included by the term “aryl”, the term “bicyclic aryl” denotes a 5-10 membered, bicyclic aryl ring which contains sufficient conjugated double bonds to form an aromatic system. One example of a bicyclic aryl is naphthyl.

Although already included under “heteroaryl”, the term “bicyclic heteroaryl” denotes a 5-10 membered, bicyclic heteroaryl ring which may contain one, two, three or four heteroatoms, selected from among oxygen, sulfur and nitrogen, and contains sufficient conjugated double bonds to form an aromatic system, unless specifically defined otherwise.

Although included by the term “bicyclic cycloalkyls” or “bicyclic aryl”, the term “fused cycloalkyl” or “fused aryl” denotes bicyclic rings wherein the bridge separating the rings denotes a direct single bond. The following are examples of a fused, bicyclic cycloalkyl:

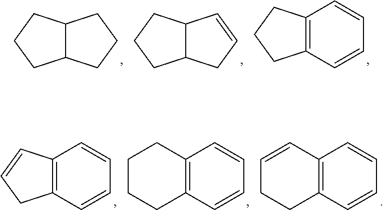

Although included by the term “bicyclic heterocycles” or “bicyclic heteroaryls”, the term “fused bicyclic heterocycles” or “fused bicyclic heteroaryls” denotes bicyclic 5-10 membered heterorings which contain one, two, three or four heteroatoms, selected from among oxygen, sulfur and nitrogen and wherein the bridge separating the rings denotes a direct single bond. The “fused bicyclic heteroaryls” moreover contain sufficient conjugated double bonds to form an aromatic system. Examples include pyrrolizine, indole, indolizine, isoindole, indazole, purine, quinoline, isoquinoline, benzimidazole, benzofuran, benzopyran, benzothiazole, benzothiazole, benzoisothiazole, pyridopyrimidine, pteridine, pyrimidopyrimidine,

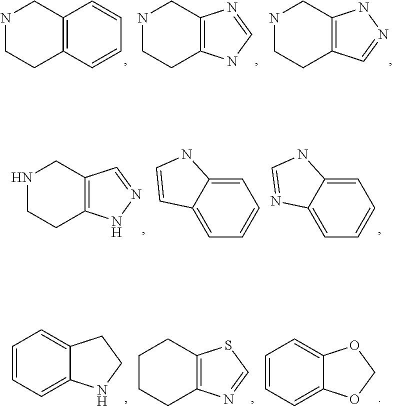

“Halogen” within the scope of the present invention denotes fluorine, chlorine, bromine or iodine. Unless stated to the contrary, fluorine, chlorine and bromine are regarded as preferred halogens.

As mentioned previously, the compounds of formulas I, II or III may be converted into the salts thereof, particularly for pharmaceutical use into the physiologically and pharmacologically acceptable salts thereof. The phrase “pharmaceutically acceptable” is employed herein to refer to those compounds, materials, compositions, and/or dosage forms which are, within the scope of sound medical judgement, suitable for use in contact with the tissue of human beings and animals without excessive toxicity, irritation, allergic response, or other problem or complication, and commensurate with a reasonable benefit/risk ratio. These salts may be present on the one hand as physiologically and pharmacologically acceptable acid addition salts of the compounds of formulas I, II or III with inorganic or organic acids. On the other hand, the compound of formulas I, II or III may be converted by reaction with inorganic bases into physiologically and pharmacologically acceptable salts with alkali or alkaline earth metal cations as counter-ion. The acid addition salts may be prepared for example using hydrochloric acid, hydrobromic acid, sulfuric acid, phosphoric acid, methanesulphonic acid, p-toluenesulfonic acid, acetic acid, fumaric acid, succinic acid, lactic acid, citric acid, tartaric acid or maleic acid. It is also possible to use mixtures of the above-mentioned acids. To prepare the alkali and alkaline earth metal salts of the compounds of formulas I, II or III it is preferable to use the alkali and alkaline earth metal hydroxides and hydrides, of which the hydroxides and hydrides of the alkali metals, particularly sodium, potassium, magnesium, calcium, zinc and diethanolamine, are preferred, while sodium and potassium hydroxide are particularly preferred.

The invention relates to the compounds in question, optionally in the form of the individual optical isomers, diastereomers, mixtures of diastereomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulfonic acid.

The compounds of formula I, II or III according to the invention may optionally be present as mixtures of diastereomeric isomers but may also be obtained as pure diastereoisomers. Preferred are the compounds with the specific stereochemistry of formulas II and III, in particular the compounds with the specific stereochemistry of formula II.

### 4 METHODS OF SYNTHESIS

General Procedure

The following methods are suitable for preparing compounds of general formulas I, II or III. The compounds according to the invention may be obtained using methods of synthesis which is are known to the one skilled in the art and described in the literature of organic synthesis. General methods for functional groups protection and deprotection steps are described e.g. in: Greene, T. W. and Wuts, P. G. M. (eds.): Protective Groups in Organic Synthesis, third edition 1999; John Wiley and Sons, Inc. Preferably the compounds are obtained analogously to the methods of preparation explained more fully hereinafter, in particular as described in the experimental section. Compounds of general formula (I) may be prepared using several alternative synthetic routes, among which the following routes shall serve as examples.

Route A:

Compounds of general formulas I, II and III, especially with A representing —CH₂— or substituted —CH₂—, can be accessed from compounds of formula (A-I) through standard amidation procedures. R13 may thereby represent hydrogen or a protecting group like e.g. tert-butyl which can be removed by standard deprotection methods. Compounds of formula (A-I) can be prepared from compounds of formula (A-II) applying standard deprotection methods. Compounds of formula (A-II) can be prepared by reacting compounds of formula (A-III) with compounds of formula (A-IV) applying a strong base like e.g. sodium hydride. Methods applicable for the preparation of compounds (A-III) become obvious to the one skilled in the art from consulting routes B and C described below and examples described in the experimental section. Compounds of formula (A-IV) can be prepared through methods described hereinafter for the syntheses of intermediates B. R according to formula (A-II) and (A-III) may either represent hydrogen or a protecting group selected from the group consisting of tert-butyl, methyl, ethyl and benzyl.

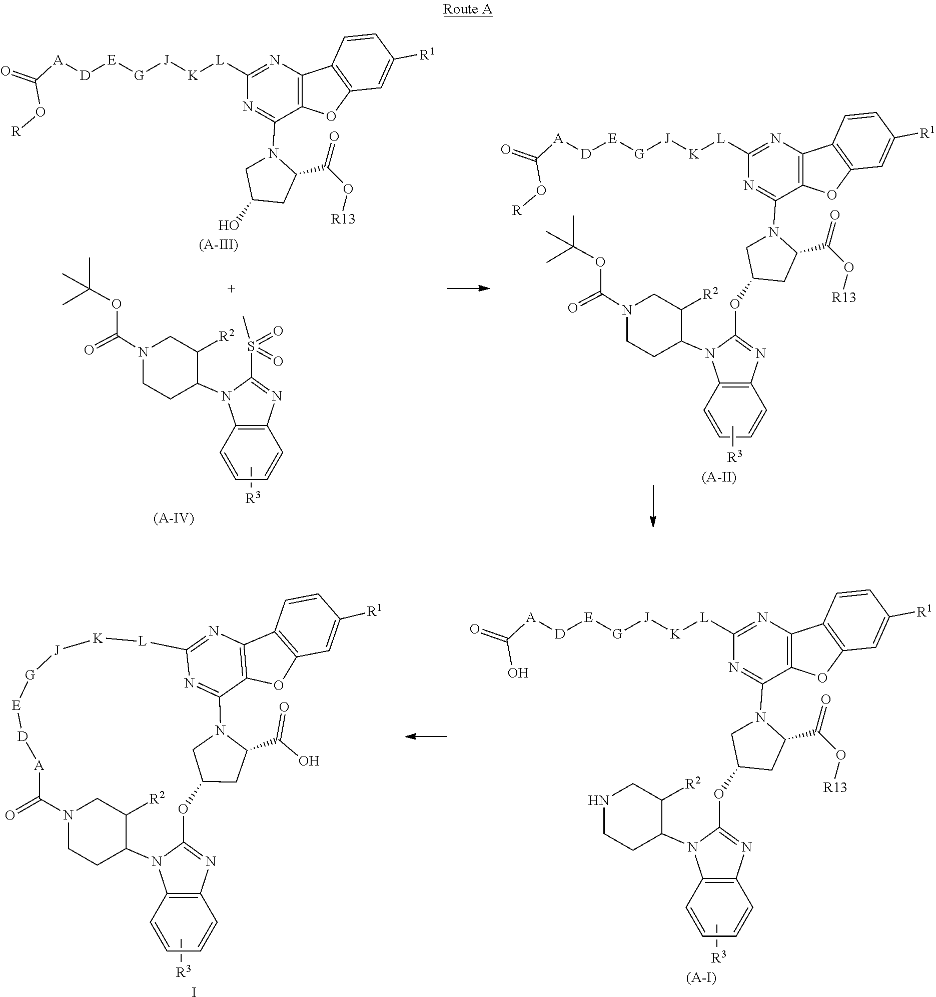

Route B:

Compounds of general formulas I, II and III can be prepared by reacting compounds of general formula (B-I) in the presence of a strong base like e.g. sodium hydride. R13 may thereby represent hydrogen or a protecting group like e.g. tert-butyl which can be removed by standard deprotection methods. Compounds (B-I) can be prepared by oxidizing compounds of general formula (B-II) applying e.g. 3-chloro-perbenzoic acid. Compounds (B-II) can be prepared by reacting compounds of general formula (B-III) with compounds of general formula (B-IV) applying standard amidation conditions. Methods applicable for the preparation of compounds (B-III) become obvious to the one skilled in the art from consulting the synthesis of compounds (B-VI) and route C described below and examples described in the experimental section. Compounds of formula (B-IV) can be prepared through methods described hereinafter for the syntheses of intermediates B. Alternatively, compounds (B-II) can be prepared by reacting compounds of general formula (B-V) with the respective enantiopure hydroxyproline, optionally in the presence of a base. Compounds (B-V) can be obtained through chlorination of compounds of general formula (B-VI) applying phosphoryl chloride. Compounds (B-VI) can be prepared from compounds of general formula (B-VII) applying strongly alkaline conditions, e.g. at elevated temperature. Compounds (B-VII) are accessible by reacting compounds of general formula (B-VIII) with compounds of general formula (B-IX) applying standard amidation procedures. Compounds of general formula (B-VIII) can be prepared through methods exemplified hereinafter or other standard methods of synthesis known to the one skilled in the art.

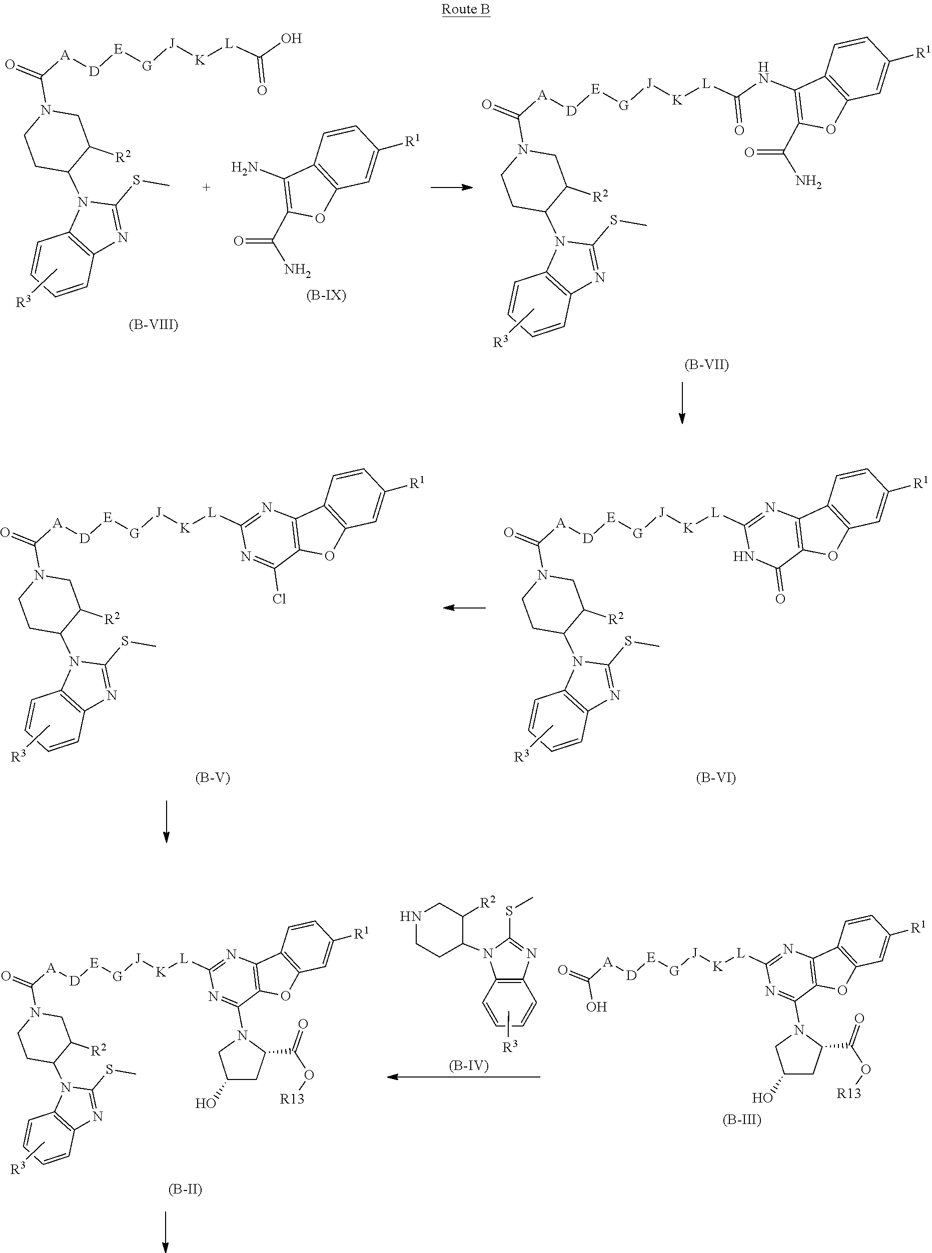

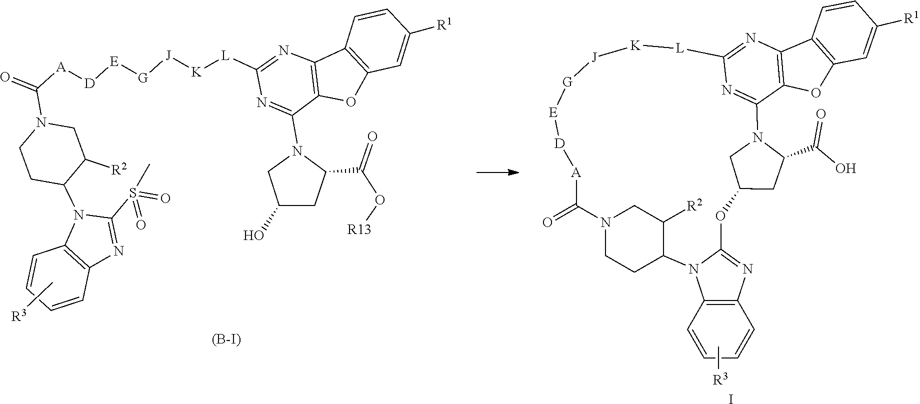

Route C:

Compounds of general formula can be prepared from compounds of general formula (C-I) by various types of ring closing reactions, as exemplified by but not restricted to: Heck type coupling reactions (with R11 representing a bromine or iodine atom and R12 containing a terminal alkene), ring closing metathesis reaction (with both R11 and R12 containing a terminal alkene) followed by hydrogenation of the resulting alkene, amidation (with R11 bearing a carboxylic acid and R12 bearing a primary or secondary amino group or vice versa). R13 may thereby represent hydrogen or a protecting group like e.g. tert-butyl which can be removed by standard deprotection methods. Methods for the synthesis of compounds (C-1) become obvious to the one skilled in the art from consulting the above routes A and B and examples described in the experimental section.

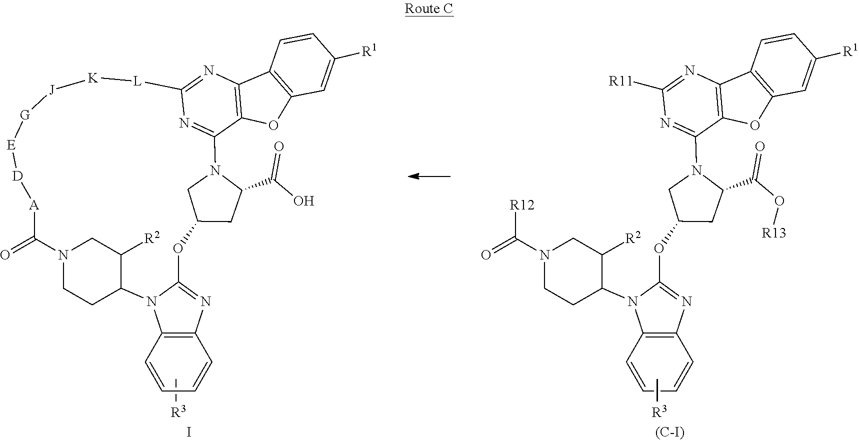

### Synthesis of Intermediates

### Intermediates A

### Intermediate A-01 (Racemic tert-butyl trans-4-amino-3-methylpiperidine-1-carboxylate)

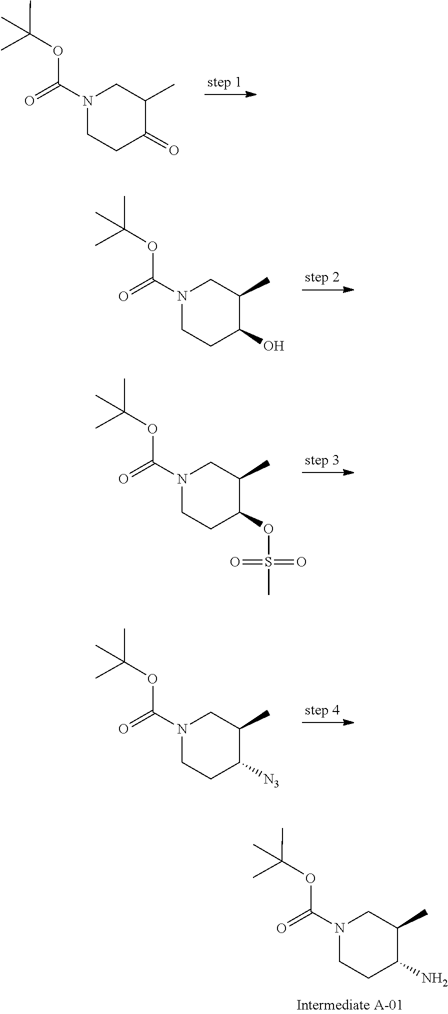

Step 1:

To a solution of tert-butyl 3-methyl-4-oxopiperidine-1-carboxylate (175 g; 0.82 mol) in THF (1.22 L), cooled to 0° C., was added lithium-tri-sec-butyl(hydrido)borate (L-selectride; 1M in THF; 984 mL; 0.98 mol). The mixture was stirred for 4 h while keeping the temperature between 0° C. and 10° C. Aq. sodium hypochlorite solution (10%; 525 mL) was added and the mixture was extracted with EtOAc (700 mL). The organic layer was separated, washed with brine, dried over sodium sulfate and evaporated. The crude product was purified by FC (silica gel; petrol ether/EtOAc 2%->50%).

¹H NMR (400 MHz, CDCl₃) δ ppm 3.84-3.82 (m, 1H), 3.52 (d, J=6.40 Hz, 2H), 3.29 (d, J=5.40 Hz, 2H), 3.06-3.02 (m, 1H), 1.76-1.74 (m, 3H), 1.65 (s, 9H), 1.24-0.89 (m, 3H)

Step 2:

To a solution of the product from step 1 (330 g; 1.53 mol) in THF (1.65 L) was added triethylamine (341 g; 3.37 mol). Methanesulfonic anhydride (507 g; 2.91 mol) was added at RT and the mixture was stirred for 4 h at RT. Water (550 mL) was added and the mixture was extracted with EtOAc (1.65 L). The organic layer was separated, washed with brine, dried over sodium sulfate and concentrated under reduced pressure. The product was taken to the next step without further purification.

¹H NMR (400 MHz, CDCl₃) δ ppm 4.74-4.71 (m, 1H), 4.23-3.95 (m, 1H), 3.17-3.04 (m, 2H), 3.04 (s, 3H), 1.95-1.92 (m, 1H), 1.83 (s, 1H), 1.83-1.82 (m, 1H), 1.32 (s, 9H), 0.91-0.79 (m, 3H)

Step 3:

Reaction under nitrogen atmosphere. To a solution of the product from step 2 (420 g; 1.43 mol) in DMF (2.1 L) was added at RT sodium azide (186 g; 2.86 mol). The mixture was stirred at 100° C. for 4 h, then cooled to 0° C. A saturated solution of sodium carbonate (4.0 L) was added while keeping the temperature below 10° C. The mixture was extracted with EtOAc, the organic layer was separated, washed with brine, dried over sodium sulfate and evaporated under reduced pressure. The product was taken to the next step without further purification.

Step 4:

A mixture of the product from step 3 (200 g; 0.83 mol), palladium on charcoal (60 g) and EtOAc (1.0 L) was stirred under hydrogen pressure (50 psi) at RT for 2 h. The catalyst was filtered off with suction and washed with methanol (2 L). The combined filtrates were evaporated under reduced pressure. The crude product was purified by FC (silica gel; petrol ether/EtOAc 0%->100%) to yield the title compound.

¹H NMR (400 MHz, CDCl₃) δ ppm 7.85 (s, 1H) 3.99-3.74 (m, 2H), 2.81-2.79 (m, 2H), 2.71 (s, 1H), 2.72-2.70 (m, 1H), 2.64-2.52 (m, 1H), 2.25-2.16 (m, 2H), 1.66-1.57 (m, 1H), 1.35-1.30 (m, 2H), 1.30-1.27 (m, 8H), 1.17-1.06 (m, 2H), 0.83-0.78 (m, 3H), 0.75-0.71 (m, 1H)

### Intermediates B

### Intermediate B-01:

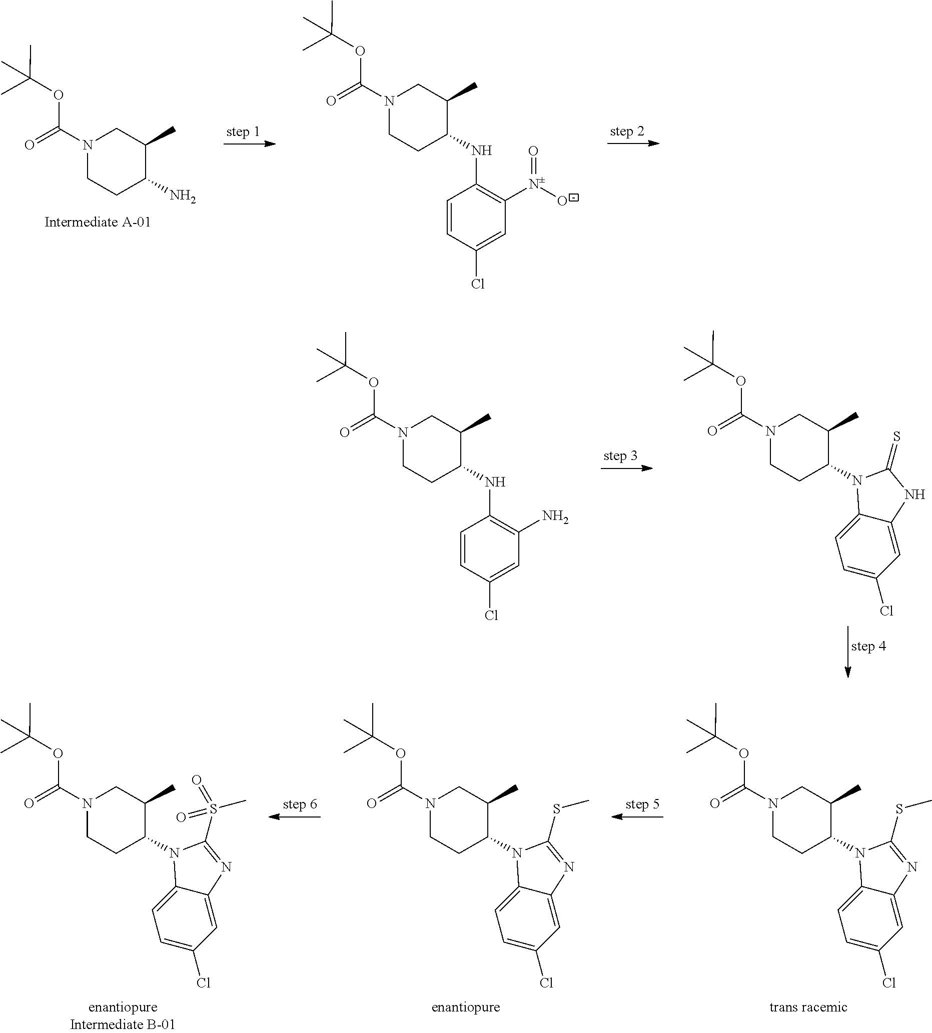

Step 1:

A mixture of intermediate A-01 (2. g; 9.97 mmol), 5-chloro-2-fluoronitrobenzene (1.21 mL; 9.97 mmol), DIPEA (3.43 mL; 19.9 mmol) and DMF (17 mL) was stirred at 60° C. until reaction control by RP HPLC indicated predominant conversion of the starting materials (10 h). The mixture was poured on ice water. Further water was added and the mixture was extracted three time with ethyl acetate. The combined organic layers were washed with water and then with brine, separated, dried over magnesium sulfate, filtered and concentrated. The crude product was purified by FC (silica gel; cyclohexane/EtOAc 20%->100%) to yield tert-butyl (rac-trans)-4-[(4-chloro-2-nitrophenyl)amino]-3-methylpiperidine-1-carboxylate.

ESI-MS: 392 [M+Na]⁺

Rₜ (HPLC): 0.83 min (method A)

Step 2:

The product from step 1 (3.25 g; 7.90 mmol), Raney nickel (410 mg) and THF (60 mL) were shaken in a Parr apparatus under hydrogen pressure (50 psi) for 5 h at RT. Further Raney nickel (160 mg) was added multiple times after further 10-15 h each, until reaction control by RP HPLC indicated high conversion of the starting material (here: catalyst addition 3 times). The catalyst was filtered off and the filtrate was evaporated. The crude product was taken to the next step.

ESI-MS: 340 [M+H]⁺

Rₜ (HPLC): 0.63 min (method A)

Step 3:

A mixture of the product from step 2 (2.16 g; 6.37 mmol), 1-(1H-imidazole-1-carbothioyl)-1H-imidazole (1.47 g; 8.25 mmol) and DMF (34 mL) was stirred at RT for 2 h. Ice water (200 mL) was added slowly to the reaction mixture while stirring and stirring was continued for further 10 min. The precipitate was filtered off, washed with water, dried over night at 50° C. and taken to the next step without further purification.

ESI-MS: 382 [M+H]⁺

Rₜ (HPLC): 0.71 min (method A)

Step 4:

Reaction under argon atmosphere. To a solution of the product from step 3 (2.57 g; 6.26 mmol) in anhydrous DMF was added potassium tert-butylate (1.42 g; 12.7 mmol). The mixture was stirred at RT for 15 min, then iodomethane (597 μL; 9.49 mmol) was added. The mixture was stirred at RT until reaction control by RP HPLC indicated near-complete conversion of the starting material (2 h). Ice water (100 mL) was added and the mixture was kept at 6° C. over night. The precipitate formed was filtered off, taken up in EtOAc, concentrated under reduced pressure and dried by co-evaporation with toluene to yield tert-butyl (racemic trans)-4-[5-chloro-2-(methylsulfanyl)-1H-1,3-benzodiazol-1-yl]-3-methylpiperidine-1-carboxylate.

ESI-MS: 396 [M+H]⁺

Rₜ (HPLC): 0.73 min (method A)

Step 5:

The mixture of enantiomers from step 4 was separated by means of preparative SFC (Instrument: Sepiatec 2 Prep SFC 100; Column: Lux Cellulose-2 (21.2 mm*250 mm, Sum); Mobile phase: A for CO2 and B for IPA; Gradient: B %=20% isocratic elution mode; Flow rate: 60 mL/min; Wavelength: 220 nm; Column temperature: 40 degrees centigrade; System back pressure: 150 bar).

The absolute configuration of the two separated enantiomers was assigned based on a co-crystal structure of example 1.01 with human cGAS protein according to methods described by D. J. Patel et al, PNAS 2019, 11946-11955 (doi.org/10.1073/pnas.1905013116).

¹H NMR (of the isomer eluting first) (400 MHz, DMSO-d₆) δ ppm 7.46-7.69 (m, 1H), 7.10-7.26 (m, 1H), 7.17 (br d, J=6.84 Hz, 1H), 3.87-4.22 (m, 3H), 2.87-3.06 (m, 1H), 2.57-2.79 (m, 4H), 2.31-2.45 (m, 1H), 2.13-2.30 (m, 1H), 1.80 (br d, J=11.79 Hz, 1H), 1.45 (s, 9H), 0.58 (d, J=6.46 Hz, 3H)

The isomer eluting first was taken to the next step. From the isomer eluting second, intermediate B-02 was prepared.

Step 6:

To a solution of the isomer eluting first from step 5 (811 mg; 2.05 mmol) in DCM (11.8 mL) was added 3-chloroperoxybenzoic acid (77%; 0.987 g; 4.40 mmol). The mixture was stirred for 2 h, then diluted with further DCM and washed with aq. potassium carbonate (15%). The aqueous layer was reextracted with DCM and the combined organic layers were washed with water, separated, dried over sodium sulfate, filtered and concentrated under reduced pressure to yield the title compound.

ESI-MS: 428 [M+H]⁺

Rₜ (HPLC): 0.74 min (method A)

The following intermediates were prepared analogously to Intermediate 1.01 described above from the starting materials indicated. Thereby, the reaction temperature in step 1 was adjusted to the reactivity of the respective nitrobenzene starting material. The absolute configuration of selected intermediates was assigned from a co-crystal structure of an example compound prepared from the respective intermediate with human cGAS protein according to methods described by D. J. Patel et al, PNAS 2019, 11946-11955 (doi.org/10.1073/pnas.1905013116). The absolute configuration of other intermediates and examples was assigned based on the assumption that eutomers share the same absolute stereochemistry in all cases.

| | Starting | | | Rₜ (HPLC) | |
|---|---|---|---|---|---|
| Int | materials | Structure | ESI-MS | (method) | Chirality comment |
| B-02 | A-01 and 5- chloro-2-fluoro- nitrobenzene | 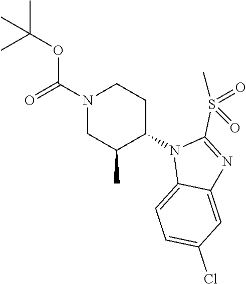 [SMILES: C[C@H]1CN(C(=O)OC(C)(C)C)CC[C@@H]1n1c(S(C)(=O)=O)nc2cc(Cl)ccc21] | 428 [M + H]⁺ | 0.74 min (A) | The isomer eluting second in step 5 was taken to step 6. |
| | | | Chiral SFC: Conditions described in Step 5 | | |
| B-03 | 4-amino-1-BOC- piperidine and 2,5-difluoro- nitrobenzene | 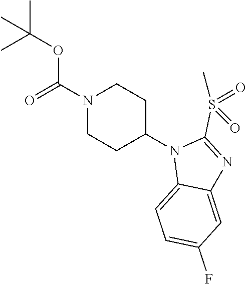 [SMILES: CC(C)(C)OC(=O)N1CCC(n2c(S(C)(=O)=O)nc3cc(F)ccc32)CC1] | 398 [M + H]⁺ | 0.66 min (A) | achiral |
| | | | achiral | | |
| B-04 | 4-amino-1-BOC- piperidine and 2,4-difluoro- nitrobenzene | 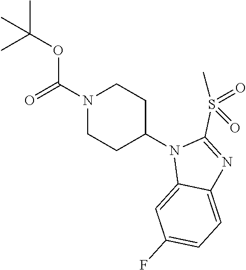 [SMILES: CC(C)(C)OC(=O)N1CCC(n2c(S(C)(=O)=O)nc3ccc(F)cc32)CC1] | 398 [M + H]⁺ | 0.66 min (A) | achiral |
| | | | achiral | | |
| B-05 | A-01 and 2- fluoro- nitrobenzene | 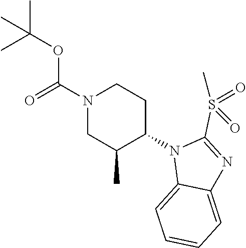 [SMILES: C[C@H]1CN(C(=O)OC(C)(C)C)CC[C@@H]1n1c(S(C)(=O)=O)nc2ccccc21] | 394 [M + H]⁺ | 0.65 min (A) | The isomer eluting first in step 5 was taken to step 6. |
| | | | Chiral SFC: EtOH(20 mM NH3)/CO2 = 10/90; | | |
| | | | column: CHIRAL ART Cellulose-SZ 20 × 250 mm, | | |
| | | | 5 μm; CT: 40° C.; BPR: 150 bar, Flow: 60 mL/min | | |
| B-06 | A-01 and 4- chloro-2-fluoro- nitrobenzene | 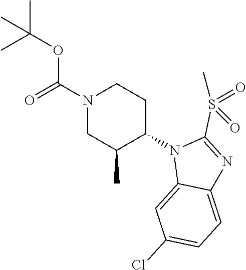 [SMILES: C[C@H]1CN(C(=O)OC(C)(C)C)CC[C@@H]1n1c(S(C)(=O)=O)nc2ccc(Cl)cc21] | 428 [M + H]⁺ | 0.74 min (A) | The isomer eluting first in step 5 was taken to step 6. |
| | | | Chiral SFC: IPA/CO2 = 10/90; column: CHIRAL | | |
| | | | ART Cellulose-SZ 20 × 250 mm, 5um; CT: 40° C.; | | |
| | | | BPR: 150 bar, Flow: 60 mL/min | | |
| B-07 | A-01 and 2,5- difluoro- nitrobenzene | 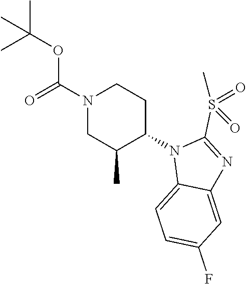 [SMILES: C[C@H]1CN(C(=O)OC(C)(C)C)CC[C@@H]1n1c(S(C)(=O)=O)nc2cc(F)ccc21] | 412 [M + H]⁺ | 0.69 min (A) | The isomers were separated after final step 6. The isomer eluting first is B-07. |
| | | | Chiral HPLC: EtOH/Heptane = 35/65; column: | | |
| | | | DAICEL Chiralpak IG 50 × 250 mm, 10 μm | | |
| B-08 | A-01 and 2- fluoro- nitrobenzene | 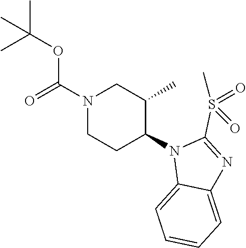 [SMILES: C[C@H]1CN(C(=O)OC(C)(C)C)CC[C@@H]1n1c(S(C)(=O)=O)nc2ccccc21] | 394 [M + H]⁺ | 0.65 min (A) | No chiral separation of enantiomers. Racemate was taken to step 6. |
| | | | racemate, trans | | |
| B-09 | A-01 and 3- chloro-2-fluoro- nitrobenzene | 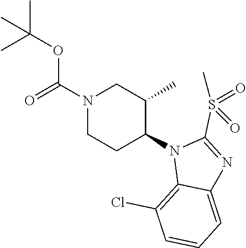 [SMILES: C[C@H]1CN(C(=O)OC(C)(C)C)CC[C@@H]1n1c(S(C)(=O)=O)nc2cccc(Cl)c21] | 428 [M + H]⁺ | 1.09 min (D) | No chiral separation of enantiomers. Racemate was taken to step 6. |
| | | | racemate, trans | | |
| B-10 | 4-amino-1-BOC- piperidine and 2-fluoro- nitrobenzene | 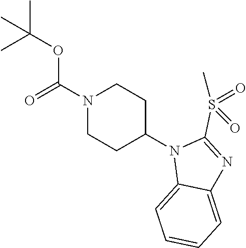 [SMILES: CC(C)(C)OC(=O)N1CCC(n2c(S(C)(=O)=O)nc3ccccc32)CC1] | 380 [M + H]⁺ | 0.62 min (A) | achiral |
| | | | achiral | | |
| B-11 | 4-amino-1-BOC- piperidine and 2,3-difluoro- nitrobenzene | 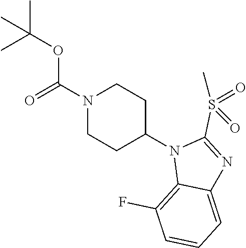 [SMILES: CC(C)(C)OC(=O)N1CCC(n2c(S(C)(=O)=O)nc3cccc(F)c32)CC1] | 398 [M + H]⁺ | 0.65 min (A) | achiral |
| | | | achiral | | |
| B-13 | A-01 and 2- fluoro-6-methyl- nitrobenzene | 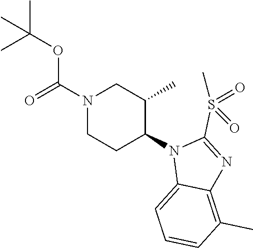 [SMILES: Cc1cccc2c1nc(S(C)(=O)=O)n2[C@H]1CCN(C(=O)OC(C)(C)C)C[C@@H]1C] | 408 [M + H]⁺ | 0.72 min (A) | No chiral separation of enantiomers. Racemate was taken to step 6. |
| | | | racemate, trans | | |

Intermediate B-12

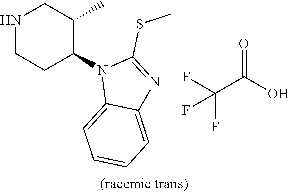

The product from step 4 of the synthesis of intermediate B-08 was BOC-deprotected according to general procedure D.

ESI-MS: 262 [M+H]⁺

Rₜ (HPLC): 0.47 min (method E)

Intermediate B-14

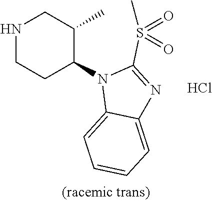 [SMILES: C[C@H]1CNCC[C@@H]1n1c(S(C)(=O)=O)nc2ccccc21 ; Cl]

A mixture of intermediate B-08 (700 mg; 1.78 mmol) and hydrochloric acid in dioxane (4 M; 1.33 mL; 5.45 mmol) was stirred for 2 h. tert-butyl methyl ether (20 mL) was added and the mixture was allowed to stand without stirring for 1 h. The precipitate was filtered off and taken up in methanol. The resulting solution was evaporated under reduced pressure to yield the title compound.

ESI-MS: 294 [M+H]⁺

Rₜ (HPLC): 0.60 min (method E)

Intermediates I

Intermediate 1.01:

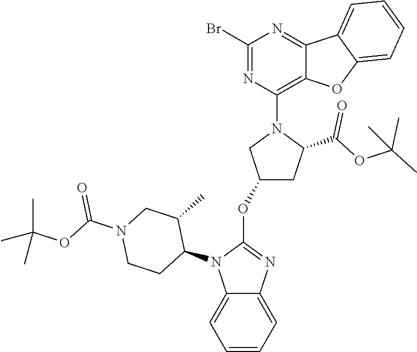 [SMILES: C[C@H]1CN(C(=O)OC(C)(C)C)CC[C@@H]1n1c(O[C@H]2C[C@@H](C(=O)OC(C)(C)C)N(c3nc(Br)nc4c3oc3ccccc34)C2)nc2ccccc21]

Intermediate N-R was reacted with intermediate B-5 according to general procedure A to yield the title compound tert-butyl (3S,4S)-4-(2-{[(3S,5S)-1-{4-bromo-8-oxa-3,5-diazatricyclo[7.4.0.0^{2,7}]trideca-1(13),2,4,6,9,11-hexaen-6-yl}-5-[(tert-butoxy)carbonyl]pyrrolidin-3-yl]oxy}-1H-1,3-benzodiazol-1-yl)-3-methylpiperidine-1-carboxylate.

ESI-MS: 747 [M+H]⁺

Rₜ (HPLC): 1.27 min (method B)

Intermediate 1.02:

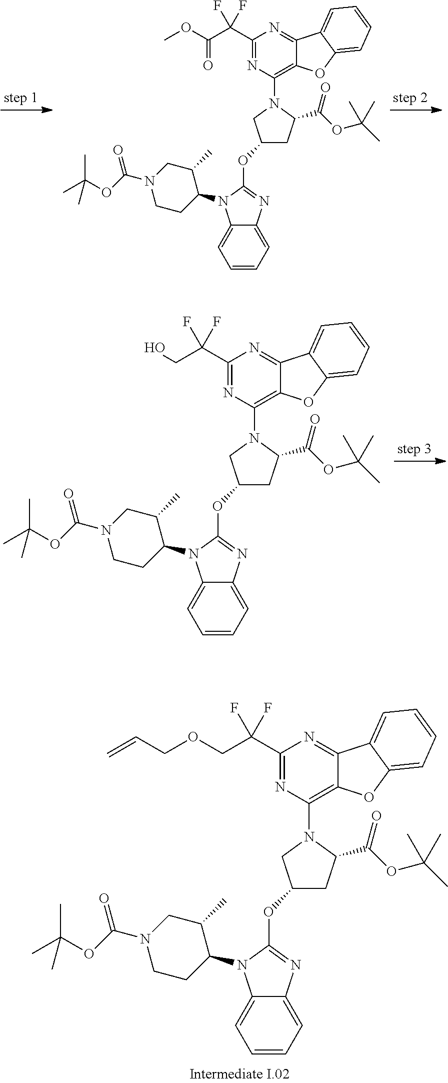

Step 1:

Reaction performed under argon atmosphere, solvent degassed and dried through addition of molecular sieves. To intermediate 1.01 (721 mg; 0.916 mmol) in a round bottomed flask was added DMSO (10 ml), methyl bromodifluoroacetate (266 μL; 2.42 mmol) and copper powder (291 mg; 4.58 mmol). The mixture was stirred overnight, then 1 further eq. of methyl bromodifluoroacetate and two further eq. of copper were added. After further 72 h stirring, the mixture was diluted with EtOAc, a solution of potassium dihydrogen phosphate (1.27 M; 20 mL) was added and the mixture was stirred for another 30 min before filtering. The organic layer of the filtrate was washed with water, concentrated and the crude product was purified by FC (silica gel; CH/EtOAc 10%->100%) to yield tert-butyl (3S,4S)-4-(2-{[(3S,5S)-5-[(tert-butoxy)carbonyl]-1-[4-(1,1-difluoro-2-methoxy-2-oxoethyl)-8-oxa-3,5-diazatricyclo[7.4.0.0^{2,7}]trideca-1(13),2,4,6,9,11-hexaen-6-yl]pyrrolidin-3-yl]oxy}-1H-1,3-benzodiazol-1-yl)-3-methylpiperidine-1-carboxylate.

Step 2:

To the product from step 1 (585 mg; 0.647 mmol) in absolute ethanol (dried over molecular sieves 3 Å) was added in portions within 1 h sodium borohydride (244 mg; 6.40 mmol). The reaction was quenched by addition of water, the mixture was extracted three times with EtOAc, and the combined organic layers were washed with water and then brine, separated and dried over magnesium sulfate, filtered and concentrated under reduced pressure. The crude product was purified by FC (silica gel; CH/EtOAc 20%->100%) to yield tert-butyl (3S,4S)-4-(2-{[(3S,5S)-5-[(tert-butoxy)carbonyl]-1-[4-(1,1-difluoro-2-hydroxyethyl)-8-oxa-3,5-diazatricyclo[7.4.0.0^{2,7}]trideca-1(13),2,4,6,9,11-hexaen-6-yl]pyrrolidin-3-yl]oxy}-1H-1,3-benzodiazol-1-yl)-3-methylpiperidine-1-carboxylate.

Step 3:

To a solution of the product from step 2 (269 mg; 0.359 mmol) and allyl bromide (220 IL; 2.52 mmol) in DMA (1.67 mL) was added sodium hydride (55% in mineral oil; 47 mg; 1.08 mmol). The mixture was stirred for 10 min, then quenched by addition of water. The mixture was extracted three times with EtOAc, and the combined organic layers were washed with water and then brine, separated and dried over magnesium sulfate, filtered and concentrated under reduced pressure. The crude product was purified by FC (silica gel; CH/EtOAc 20%->100%) to yield the title compound tert-butyl (3S,4S)-4-(2-{[(3S,5S)-5-[(tert-butoxy)carbonyl]-1-{4-[1,1-difluoro-2-(prop-2-en-1-yloxy)ethyl]-8-oxa-3,5-diazatricyclo[7.4.0.0^{2,7}]trideca-1(13),2,4,6,9,11-hexaen-6-yl}pyrrolidin-3-yl]oxy}-1H-1,3-benzodiazol-1-yl)-3-methylpiperidine-1-carboxylate.

ESI-MS: 789 [M+H]⁺

Rₜ (HPLC): 1.29 min (method B)

Intermediate 1.03:

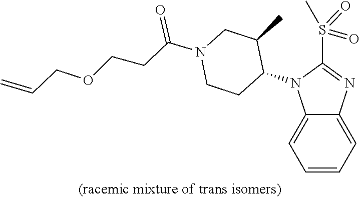 [SMILES: C=CCOCCC(=O)N1CC[C@@H](n2c(S(C)(=O)=O)nc3ccccc32)[C@H](C)C1]

To a solution of 3-allyloxypropionic acid (38 IL; 0.303 mmol) in DMF (500 IL) was added EDC hydrochloride (70 mg; 0.364 mmol) and N,N-diisopropylethylamine (105 μL; 0.606 mmol). The mixture was stirred for 10 min, then intermediate B-14 (100 mg; 0.303 mmol) was added and the mixture was stirred for 90 min, then diluted with methanol (2 mL) and subjected to purification by RP HPLC (Sunfire C18; ACN/water, modifier: TFA).

ESI-MS: 406 [M+H]⁺

Rₜ (HPLC): 0.88 min (method E)

Intermediates N

The syntheses described hereinafter were in part carried out according to the following general procedures indicated.
General procedure Int-A: Hydrogenation with Pd/C catalyst (see: Intermediate N-A, step 4)
General procedure Int-B: Ring closure under basic conditions (see: Intermediate N-A, step 5)
General procedure Int-C: Chlorination applying phosphoroyl trichloride (see: Intermediate N-A, step 7)
General procedure Int-D: S_{N}Ar with hydroxyproline ester (see: Intermediate N-A, step 8)
General procedure Int-E: Ester cleavage applying lithium hydroxide (see: Intermediate N-F, step 2)
General procedure Int-F: Amidation applying PFTU (see: Intermediate N-F, step 3)
General procedure Int-G: Heck type coupling (see: Intermediate N-J, step 1)

Intermediate N-A:

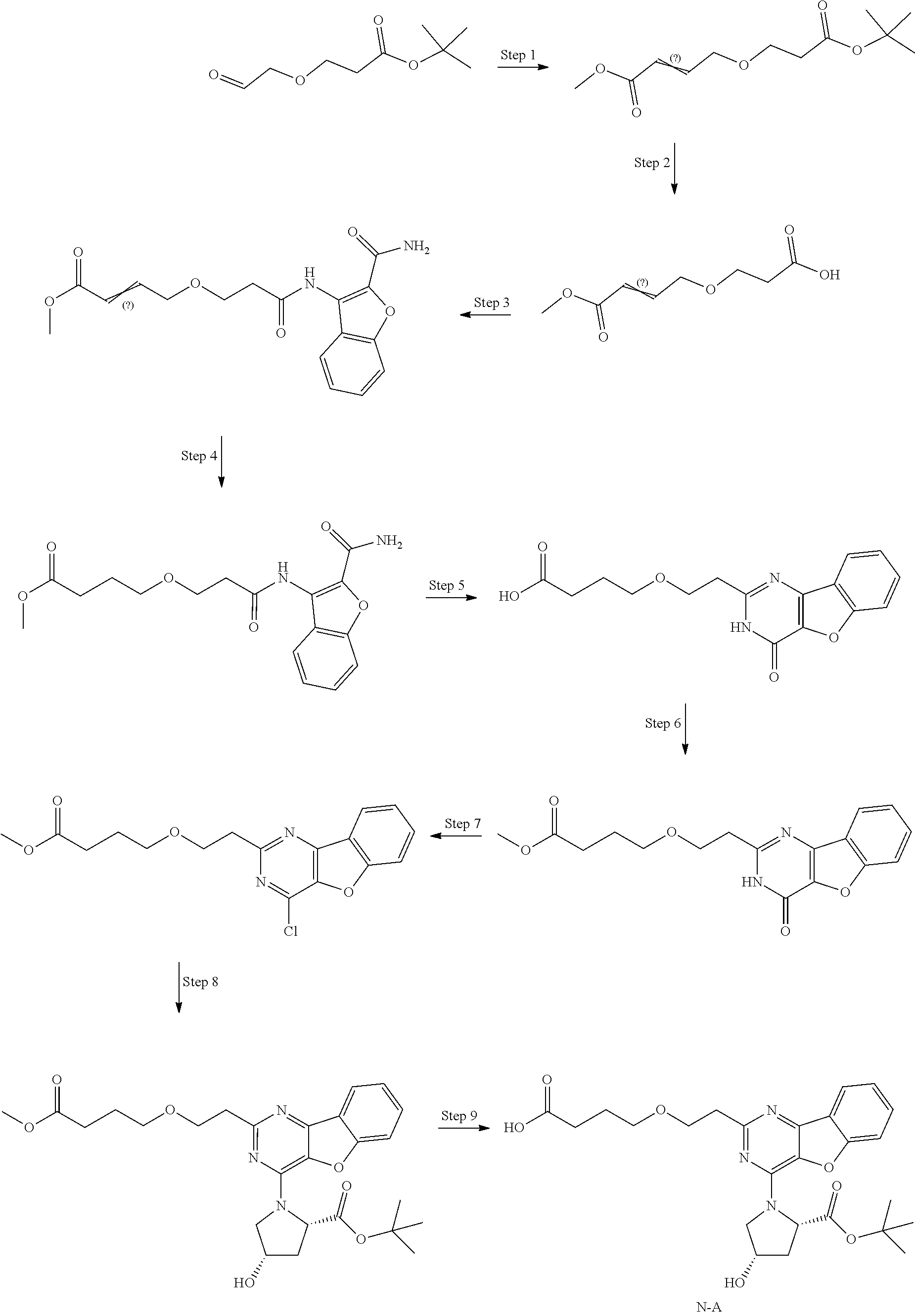

Step 1:

A mixture of tert-butyl 3-(2-oxoethoxy)propanoate (7.25 g; 38.5 mmol) which was prepared as described in EP2409977, and methyl (triphenylphosphoranylidene)acetate (13.1 g; 38.5 mmol) in DCM (200 mL) was stirred at RT over night. The mixture was evaporated under reduced pressure and taken up in CH/EtOAc (3:1). Insolubles were removed by filtration and the filtrate was evaporated. The crude product was purified by FC (silica gel; CH/EtOAc 10%->45%) to yield the product as a mixture of cis- and trans-isomers.

Step 2:

The product from step 1 (1.50 g; 6.14 mmol) was stirred in a mixture of DCM (15 mL) and TFA (10 mL) over night. The mixture was evaporated and taken up in methanol (10 mL). Polymer bound tetraalkylammoniumcarbonate (2 weight equivalents) was added and the mixture was stirred for 90 min. Insolubles were filtered off and the filtrate was evaporated.

Step 3:

To a solution of the product from step 2 (5.00 g; 21.3 mmol) in ACN (140 mL) was added 1-chloro-N,N,2-trimethylpropenylamine (4.45 mL; 33.6 mmol). The mixture was stirred at RT for 10 min, then pyridine (5.10 mL; 63.0 mmol) and 3-aminobenzofuran-2-carboxamide (3.70 g; 21.0 mmol) was added.

The mixture was stirred over night at RT, then water was added. The mixture was extracted with DCM and the organic layer was separated and evaporated. The crude product was purified first by FC (silica gel; petrol ether/EtOAc 40%->80%) and second by RP HPLC (Sunfire C18, ACN/water, modifier: TFA).

ESI-MS: 347 [M+H]⁺

Rₜ (HPLC): 0.80 min (method E)

Step 4: General procedure Int-A:

A mixture of the product from step 3 (3.40 g; 9.73 mmol), palladium on charcoal (10%; 350 mg) and ethanol (500 mL) was shaken under hydrogen pressure (50 psi) until reaction control by RP HPLC indicated conversion of the starting material (here: 90 min). The catalyst was filtered off and the filtrate was evaporated to dryness.

ESI-MS: 349 [M+H]⁺

Rₜ (HPLC): 0.81 min (method E)

Step 5:

General procedure Int-B:

A mixture of the product from step 4 (4.55 g; 13.1 mmol) and aq. sodium hydroxide (4 M; 100 mL; 400 mmol) was stirred at 60° C. until reaction control by RP HPLC indicated conversion of the starting material (here: 60 min). The mixture was allowed to cool to RT and was then acidified by addition of aq. hydrochloric acid (4 M). The precipitated was collected and dried at 60° C.

ESI-MS: 317 [M+H]⁺

Rₜ (HPLC): 0.73 min (method E)

Step 6:

To a mixture of the product from step 5 (3.53 g; 11.2 mmol), DCM (60 mL), and a few drops of DMF was added at RT oxalyl chloride (1.24 mL; 14.5 mmol). The mixture was stirred for 3 h at RT, then methanol was added and stirring was continued for another 60 min. The mixture was extracted with water and the organic layer was separated and evaporated to dryness.

ESI-MS: 331 [M+H]⁺

Rₜ (HPLC): 0.82 min (method E)

Step 7:

General procedure Int-C:

A mixture of the product from step 6 (3.70 g; 11.2 mmol) and phosphoroyl trichloride (70 mL) was stirred at 90° C. for 4 h. Surplus phosphoroyl trichloride was removed by distillation and water was added carefully. The resulting mixture was extracted with EtOAc, the organic layer was separated and evaporated. The crude product was taken to the next step.

ESI-MS: 349 [M+H]⁺

Rₜ (HPLC): 1.02 min (method E)

Step 8:

General procedure Int-D:

A mixture of the product from step 7 (300 mg; 0.896 mmol), tert-butyl (2S,4S)-4-hydroxypyrrolidine-2-carboxylate hydrochloride (253 mg; 1.08 mmol), potassium carbonate (300 mg; 2.06 mmol) and DMF (7.0 mL) was stirred at RT over night. Water was added and the mixture was acidified by addition of aq. hydrochloric acid (1 M). The mixture was extracted with EtOAc, the organic layer was evaporated and the crude product was purified by FC (silica gel; petrol ether/EtOAc 40%->75%)

ESI-MS: 500 [M+H]⁺

Rₜ (HPLC): 0.75 min (method E)

Step 9:

The product from step 8 was reacted according to the general procedure Int-E to afford the ester cleaved title compound.

ESI-MS: 486 [M+H]⁺

Rₜ (HPLC): 0.70 min (method E)

Intermediate N-B:

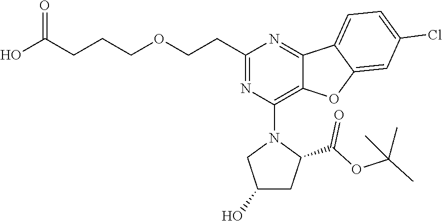 [SMILES: CC(C)(C)OC(=O)[C@@H]1C[C@H](O)CN1c1nc(CCOCCCC(=O)O)nc2c1oc1cc(Cl)ccc12]

Was prepared analogously to the sequence described for the synthesis of intermediate N-A, applying 3-amino-6-chloro-1-benzofuran-2-carboxamide in step 3.

ESI-MS: 520 [M+H]⁺

Rₜ (HPLC): 0.50 min (method A)

Intermediate N-C:

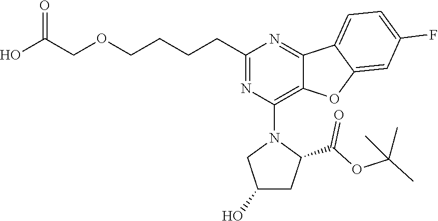 [SMILES: CC(C)(C)OC(=O)[C@@H]1C[C@H](O)CN1c1nc(CCCCOCC(=O)O)nc2c1oc1cc(F)ccc12]

Preparation was performed from intermediate N-V and 2-(but-3-en-1-yloxy)acetic acid applying a two step sequence:
Step 1: according to general procedure Int-G
Step 2: according to general procedure Int-A to yield the title compound.

ESI-MS: 504 [M+H]⁺

Rₜ (HPLC): 0.45 min (method A)

Intermediate N-D:

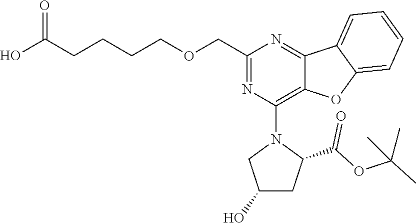 [SMILES: CC(C)(C)OC(=O)[C@@H]1C[C@H](O)CN1c1nc(COCCCCC(=O)O)nc2c1oc1ccccc12]

Was prepared analogously to the sequence described for the synthesis of intermediate N-A, starting from tert-butyl 2-(3-oxopropoxy)acetate which was prepared as described in EP1939201.

ESI-MS: 486 [M+H]⁺

Rₜ (HPLC): 0.71 min (method E)

Intermediate N-E:

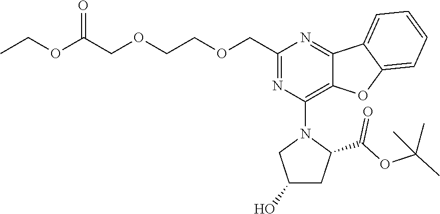 [SMILES: CCOC(=O)COCCOCc1nc(N2C[C@@H](O)C[C@H]2C(=O)OC(C)(C)C)c2oc3ccccc3c2n1]

Step 1:

A mixture of 3-aminobenzofuran-2-carboxamide (2.00 g; 11.4 mmol), ethyl 2-[2-(2-ethoxy-2-oxoethoxy)ethoxy]acetate (5.32 g; 22.7 mmol) and 1H,2H,3H,4H,6H,7H,8H-[1,3]diazino[1,2-a]pyrimidine (6.45 g; 45.4 mmol) was heated to 120° C. for 150 min. After cooling to RT, aq. hydrochloric acid (1M; 70 mL) was added. The precipitate was filtered off with suction, washed with water and dried in vacuo at 60° C. to yield a mixture of ethyl 2-[2-({6-oxo-8-oxa-3,5-diazatricyclo[7.4.0.0^{2,7}]trideca-1(9),2(7),3,10,12-pentaen-4-yl}methoxy)ethoxy]acetate and the respective free acid which was taken to the next step.

Step 2:

For reesterification of the acid, the mixture from step 1 (2.8 g) was dissolved in DCM (200 mL). 2 drops of DMF were added, followed by the addition of oxalyl chloride (392 μL, 4.57 mmol). The mixture was stirred over night, then ethanol (10 mL) was added, and the mixture stirred for further 2 h. The mixture was concentrated under reduced pressure. Methyl tert-butyl ether was added and the precipitate formed was washed with methyl tert-butyl ether and dried at 50° C. to yield ethyl 2-[2-({6-oxo-8-oxa-3,5-diazatricyclo[7.4.0.0^{2,7}]trideca-1(9),2(7),3,10,12-pentaen-4-yl}methoxy)ethoxy]acetate.

The product from step 2 was further reacted in a 2 step sequence according to first general procedure Int-C and then Int-D to yield the title compound.

ESI-MS: 516 [M+H]⁺

Rₜ (HPLC): 0.80 min (method E)

Intermediate N-F:

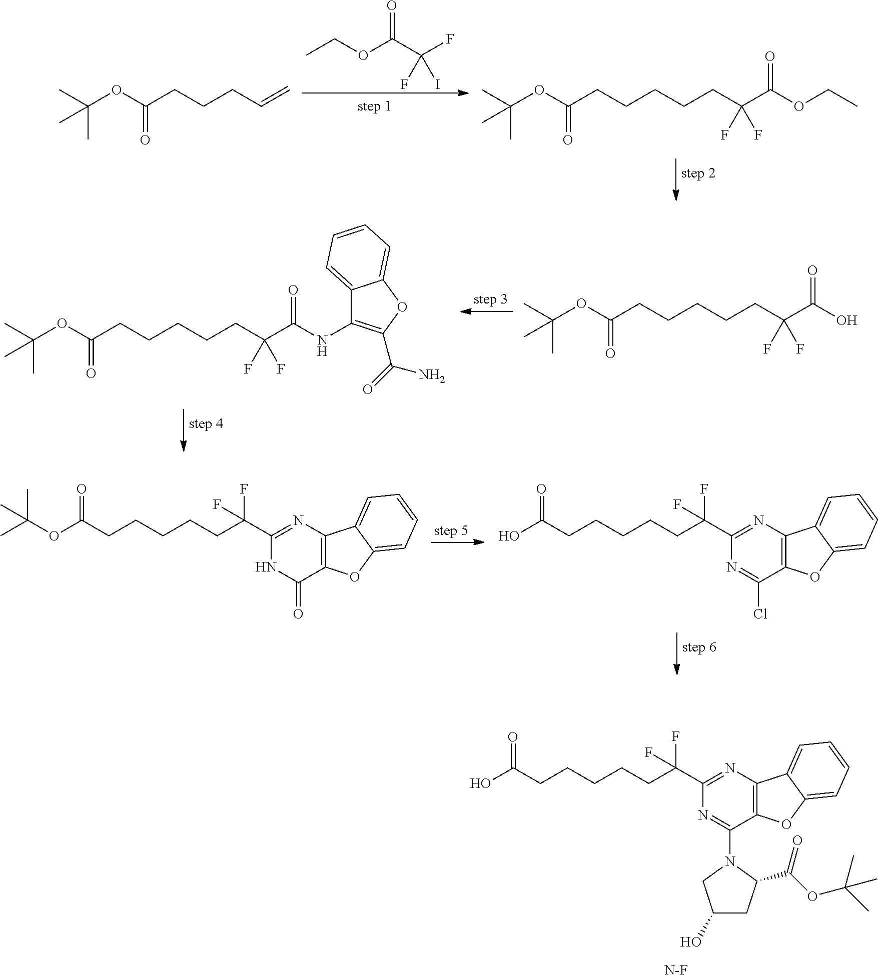

Step 1:

The zinc powder used was washed with 2% aq. hydrochloric acid, water and acetone prior to use. The purified powder was dried in high vacuum and stored under argon. A flask was charged with zinc powder (2.44 g; 36.9 mmol), nickel(II)chloride hexahydrate (0.754 g; 3.14 mmol), THF (35.0 m L) and 3 drops of water and the mixture was stirred for 10 min at RT. Then, tert-butyl hex-5-enoate (4.63 g; 18.5 mmol; prepared as described in WO2010/15447) was added in one portion and ethyl iodofluoroacetate (2.80 mL; 18.5 mmol) was added dropwise (exothermic reaction during addition) while the temperature was kept below 30° C. After completed addition, the reaction mixture was stirred for 4 h at 60° C. The reaction mixture was poured into a mixture of sat. ammonium chloride solution (100 mL) and diethyl ether (100 mL) and stirred for 10 min. Then, the mixture was filtered through a pad of celite and after phase separation, the aqueous phase was extracted with diethyl ether. The combined organic layers were washed with water, dried with sodium sulfate, filtered and evaporated. The crude product was purified by FC (silica gel; CH/DCM 10%->100%).

ESI-MS: 312 [M+NH₄]⁺

Rₜ (HPLC): 0.78 min (method A)

Step 2:

General Procedure Int-E:

Lithium hydroxide (276 mg; 11.0 mmol) was added to a solution of the product from step 1 in 2:1 THF/H₂O and the reaction mixture was stirred at RT until reaction control by RP HPLC indicated consumption of the starting material (here: 2.5 h). Volatiles were removed in vacuo, the residue was acidified to pH=1 by addition of 5.50 mL of 1N aq. hydrochloric acid and the mixture was extracted with EtOAc thrice. The combined organic phase was dried with sodium sulfate, filtered and evaporated.

ESI-MS: 211 [M-isobutene+H]⁺

Rₜ (HPLC): 0.59 min (method A)

Step 3:

General Procedure Int-F:

At RT, PFTU (2.40 g; 5.60 mmol) was added to a stirred solution of the product from step 2 (1.40 g; 5.09 mmol) and DIPEA (970 μL; 5.60 mmol) in DMF (21.0 mL) and the mixture was stirred for 30 min.

Then, 3-aminobenzofuran-2-carboxamide (1.01 g; 5.60 mmol) and further DIPEA (970 μL; 5.60 mmol) were added and the mixture was stirred for 10 min at RT. Then, the reaction mixture was heated to 50° C. and stirred for 16 h at this temperature. As reaction control by RP HPLC indicated incomplete consumption of starting materials, additional DIPEA (441 μL; 2.80 mmol) and PFTU (0.86 g; 2.04 mmol) were added and stirring at 50° C. was continued for an additional 2.5 h. The reaction mixture was diluted with water, acidified with TFA, filtered and purified by means of RP HPLC (XBridge C18; ACN/water; modifier: TFA).

ESI-MS: 425 [M+H]⁺

Rₜ (HPLC): 0.72 min (method A)

Step 4:

At RT, chlorotrimethylsilane (4.05 mL; 30.3 mmol) was added slowly to a solution of the product from step 3 (950 mg; 2.13 mmol and TEA (13.0 mL; 92.4 mmol) in 1,2-dichloroethane (28.5 mL). After completed addition, the reaction mixture was heated to 85° C. and stirred for 24 h at this temperature. The reaction mixture was poured into 30 mL 4 M hydrochloric acid (pH=1) and extracted with DCM twice. The combined organic phase was washed with water, dried with sodium sulfate, filtered and evaporated to afford tert-butyl 7,7-difluoro-7-{6-oxo-8-oxa-3,5-diazatricyclo[7.4.0.0^{2,7}]trideca-1(13),2(7),3,9,11-pentaen-4-yl}heptanoate.

ESI-MS: 405 [M−H]⁻

Rₜ (HPLC): 0.44 min (method F)

Step 5:

The product from step 4 was converted to the chlorinated product applying general procedure Int-C, followed by purification by RP-HPLC (XBridge C18; ACN/water; modifier: TFA)

ESI-MS: 367 [M−H]⁻

Rₜ (HPLC): 0.64 min (method A)

Step 6:

The product from step 6 was reacted according to general procedure Int-D to yield the title compound.

ESI-MS: 520 [M+H]⁺

Rₜ (HPLC): 0.61 min (method A)

Intermediate N-G:

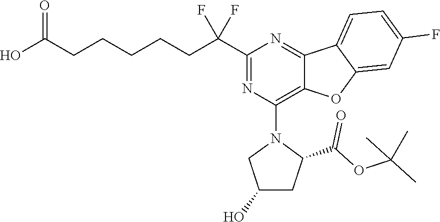 [SMILES: CC(C)(C)OC(=O)[C@@H]1C[C@H](O)CN1c1nc(C(F)(F)CCCCCC(=O)O)nc2c1oc1cc(F)ccc12]

Preparation analogously to the reaction sequence described for the synthesis of intermediate N-F, applying 3-amino-6-fluorobenzofuran-2-carboxamide in step 3.

Intermediate N-H:

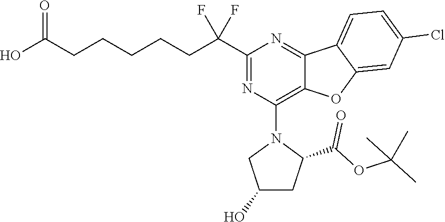 [SMILES: CC(C)(C)OC(=O)[C@@H]1C[C@H](O)CN1c1nc(C(F)(F)CCCCCC(=O)O)nc2c1oc1cc(Cl)ccc12]

Preparation analogously to the reaction sequence described for the synthesis of intermediate N-F, applying 3-amino-6-chlorobenzofuran-2-carboxamide in step 3.

Intermediate N-I:

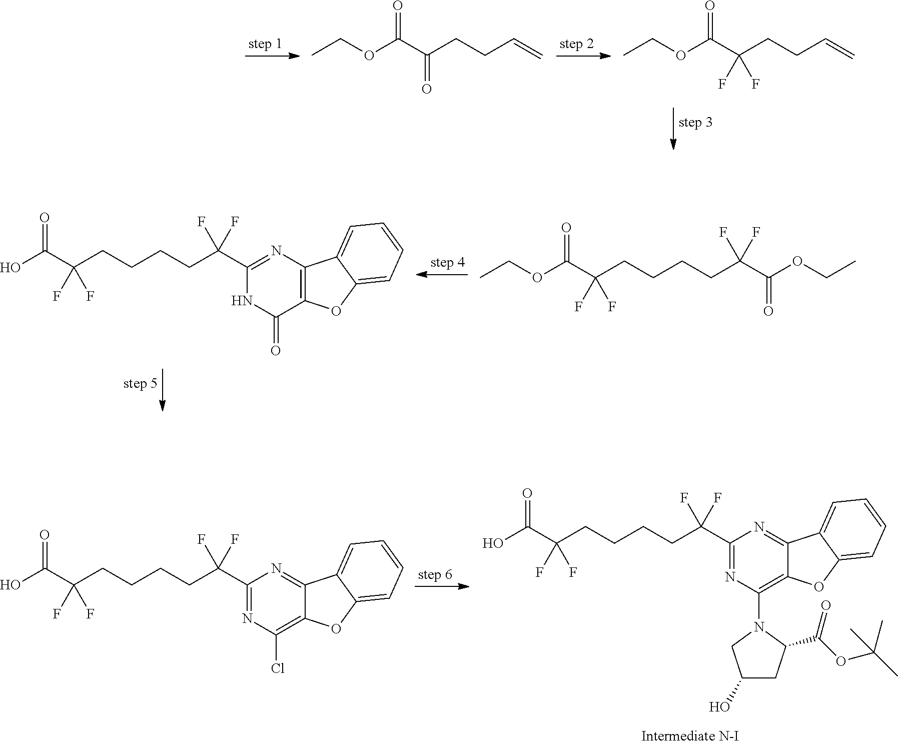 [SMILES: C=CCCC(=O)C(=O)OCC ; C=CCCC(F)(F)C(=O)OCC ; O=C(O)C(F)(F)CCCCC(F)(F)c1nc2c(oc3ccccc32)c(=O)[nH]1 ; CCOC(=O)C(F)(F)CCCCC(F)(F)C(=O)OCC ; O=C(O)C(F)(F)CCCCC(F)(F)c1nc(Cl)c2oc3ccccc3c2n1 ; CC(C)(C)OC(=O)[C@@H]1C[C@H](O)CN1c1nc(C(F)(F)CCCCC(F)(F)C(=O)O)nc2c1oc1ccccc12]

Step 1:

To a solution of magnesium powder (45.0 g; 1.85 mol) in THF (285 mL), was added Iodine (1.00 g; 3.94 mmol), followed by dropwise addition of a solution of 1-bromo-3-butene (111 g; 821 mmol) in THE (850 mL). The temperature was thereby kept below 50° C. The resulting mixture was cooled to −75° C. and a solution of diethyl oxalate (100 g; 684 mmol; 93.5 mL) in THF (1.89 L) was dropwise added at −75° C. The mixture was stirred at −75° C. for further 4 h. The reaction mixture was quenched by addition saturated aq. ammonium chloride solution (900 mL) at 0° C., then the pH was adjusted to pH3 by addition of aq. hydrochloric acid (1 M). The mixture was extracted three times with EtOAc (500 mL). The combined organic layers were washed with brine (900 mL), dried over sodium sulfate, filtered and concentrated under reduced pressure to give a residue which was purified by FC (silica gel; Petroleum ether/Ethyl acetate 0%->100%).

¹H NMR: (400 MHz, CDCl₃) δ=5.85-5.72 (m, 1H), 5.11-4.92 (m, 2H), 4.37-4.24 (m, 2H), 2.92 (t, J=7.3 Hz, 2H), 2.36 (q, J=7.1 Hz, 2H), 1.24-1.21 (m, 3H) Step 2:

To a solution the product from step 1 (50.0 g, 320 mmol) in DCM (1000 mL) was added Bis-(2-methoxyethyl)aminosulfor trifluoride (Deoxofluor) (120 g, 544 mmol, 119 mL) and ethanol (2.95 g, 64.0 mmol) at 0° C. The mixture was stirred at 25° C. for 12 h. The reaction mixture was quenched by addition of 500 mL saturated aq. sodium bicarbonate and then extracted three times with DCM (500 mL). The combined organic layers were washed with aq. hydrochloric acid (1 M; 200 mL) and brine (200 mL), dried over sodium sulfate, filtered and concentrated under reduced pressure to give a residue. The crude product was distilled in vacuo (30° C., −0.09 MPa/oil pump).

¹H NMR: (400 MHz, CDCl₃): δ=5.80 (br dd, J=10.3, 16.9 Hz, 1H), 5.13-5.00 (m, 2H), 4.39-4.28 (m, 2H), 2.30-2.12 (m, 4H), 1.43-1.34 (m, 3H) Step 3:

To a mixture of zinc powder (8.79 g, 134 mmol) and THF (30.0 mL) was added nickel(II)chloride hexahydrate (804 mg, 3.38 mmol). The mixture was stirred at −65° C. for 5 min. Then was added dropwise ethyl difluoroiodoacetate (12.0 g, 48.0 mmol) and the product from step 2 (6.00 g, 33.7 mmol) at −65° C. The mixture was stirred at 25° C. for 12 h. The reaction mixture was quenched by addition saturated aq. ammonium chloride (60.0 mL) at 0° C., and then extracted three times with DCM (60.0 mL). The combined organic layers were washed with brine (60.0 mL), dried over sodium sulfate, filtered and concentrated under reduced pressure.

¹H NMR: (400 MHz, chloroform-d): δ=4.33 (q, J=7.2 Hz, 4H), 2.18-1.98 (m, 4H), 1.56 (td, J=3.8, 8.0 Hz, 4H), 1.36 (t, J=7.2 Hz, 6H) Step 4:

To a solution of the product from step 3 (11.0 g, 36.4 mmol) in dioxane (30.0 mL) was added 1,5,7-Triazabicyclo[4.4.0]dec-5-ene (14.2 g, 102 mmol) and 3-amino-1-benzofuran-2-carboxamide (4.50 g, 25.5 mmol). The mixture was stirred at 110° C. for 2 hrs and then was diluted with water (30.0 mL) and extracted three times with EtOAc (30.0 mL). The combined organic layers were washed with brine (30.0 mL), dried over sodium sulfate, filtered and concentrated under reduced pressure. The crude product was purified by RP HPLC (ACN/water, modifier: TFA).

¹H NMR: (400 MHz, DMSO-d): δ=8.09 (d, J=7.7 Hz, 1H), 7.88 (d, J=8.4 Hz, 1H), 7.72 (t, J=7.8 Hz, 1H), 7.53 (t, J=7.5 Hz, 1H), 2.40-2.37 (m, 2H), 2.12-2.07 (m, 2H), 1.54-1.48 (m, 4H).

Step 5:

The product from step 4 was chlorinated applying general procedure Int-C.

Step 6:

The product from step 5 was reacted applying general procedure Int-D to yield the title compound.

ESI-MS: 556 [M+H]⁺

Rₜ (HPLC): 0.64 min (method A)

Intermediate N-J:

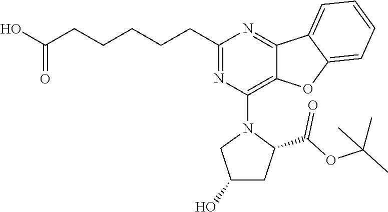 [SMILES: CC(C)(C)OC(=O)[C@@H]1C[C@H](O)CN1c1nc(CCCCCC(=O)O)nc2c1oc1ccccc12]

Preparation was performed from intermediate N-R applying the following 3 step sequence:

Step 1:

General procedure Int-G:

Intermediate N-R (300 mg; 0.69 mmol) and 5-hexenoic acid methyl ester (298 IL; 2.07 mmol) were dissolved in DMF (10 mL; 123 mmol). Triethylamine (0.39 mL; 2.76 mmol) was added and the mixture was degassed with argon. Palladium(II)-acetate (31 mg; 0.14 mmol) and tri-o-tolylphosphine (84 mg; 0.28 mmol) were added under argon. The sealed vial was stirred at 95° C. over night. The mixture was diluted with ACN/water, acidified with TFA, filtered over a syringe-filter. Purification by means of prep. RP HPLC (Sunfire C18, ACN/water; modifier: TFA) yielded tert-butyl (2S,4S)-4-hydroxy-1-{4-[6-methoxy-6-oxohex-1-en-1-yl]-8-oxa-3,5-diazatricyclo[7.4.0.0^{2,7}]trideca-1(13),2,4,6,9,11-hexaen-6-yl}pyrrolidine-2-carboxylate.

Step 2:

The product from step 1 was hydrogenated according to general procedure Int-A to yield tert-butyl (2S,4S)-4-hydroxy-1-[4-(6-methoxy-6-oxohexyl)-8-oxa-3,5-diazatricyclo[7.4.0.0^{2,7}]trideca-1(13),2,4,6,9,11-hexaen-6-yl]pyrrolidine-2-carboxylate.

ESI-MS: 484 [M+H]⁺

Rₜ (HPLC): 0.65 min (method B)

Step 3:

The product from step 2 was reacted according general procedure Int-E to afford the ester cleaved title compound.

Intermediate N-K:

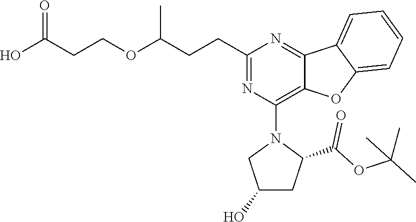 [SMILES: CC(CCc1nc(N2C[C@@H](O)C[C@H]2C(=O)OC(C)(C)C)c2oc3ccccc3c2n1)OCCC(=O)O]

Preparation was performed from intermediate N-R and methyl 3-(but-3-en-2-yloxy)propanoate applying the following three step sequence (The starting material methyl 3-(but-3-en-2-yloxy)propanoate was prepared from the respective tert-butyl ester by acidic ester cleavage followed by methyl ester formation (methanol, thionyl chloride):
Step 1: Heck type coupling according to general procedure Int-G
Step 2: Hydrogenation according to general procedure Int-A
Step 3: Ester cleavage according to general procedure Int-E to yield the title compound.

ESI-MS: 500 [M+H]⁺

Rₜ (HPLC): 1.82 min (method G)

Intermediate N-L:

### 2-(4-{6-[(2S,4S)-2-[(tert-butoxy)carbonyl]-4-hydroxypyrrolidin-1-yl]-8-oxa-3,5-diazatricyclo[7.4.0.0^{2,7}]trideca-1(13),2,4,6,9,11-hexaen-4-yl}-4,4-difluorobutoxy)acetic acid

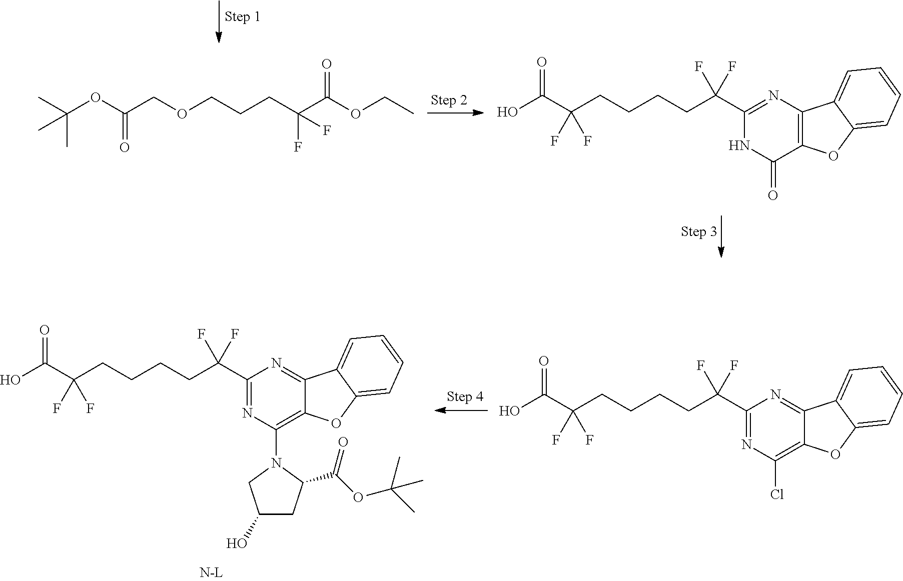 [SMILES: CCOC(=O)C(F)(F)CCCOCC(=O)OC(C)(C)C ; O=C(O)C(F)(F)CCCCC(F)(F)c1nc2c(oc3ccccc32)c(=O)[nH]1 ; CC(C)(C)OC(=O)[C@@H]1C[C@H](O)CN1c1nc(C(F)(F)CCCCC(F)(F)C(=O)O)nc2c1oc1ccccc12 ; O=C(O)C(F)(F)CCCCC(F)(F)c1nc(Cl)c2oc3ccccc3c2n1]

Step 1:

A dry reaction vessel was equipped with a magnetic stir bar and charged with tert-butyl 2-allyloxyacetate (1.00 g; 5.81 mmol), anhydrous nickel(II)chloride (0.038 g; 0.290 mmol) and sodium carbonate (0.61 g; 5.81 mmol). The reaction vessel was then briefly evacuated and backfilled with argon (this sequence was repeated a total of three times). Anhydrous DMF (40 mL), ethyl bromodifluoroacetate (1.5 mL, 11.6 mmol) and phenylsilane (2.9 mL; 23.2 mmol) were added to the reaction vessel via syringe sequentially. The vessel was heated at 70° C. in an oil-bath anf stirred until TLC monitoring indicated consumption of the starting material (here: over night). The reaction mixture was diluted with 30 mL of EtOAc, and the organic layer was washed with 80 mL of saturated aqueous sodium chloride solution. After that, the organic layer was dried over sulfate and concentrated under reduced pressure and purified further by FC (silica gel; hexane/EtOAc) to yield ethyl 5-(2-tert-butoxy-2-oxo-ethoxy)-2,2-difluoro-pentanoate.

Step 2:

At RT, 1,5,7-Triazabicyclo[4.4.0]dec-5-ene (0.79 g; 5.68 mmol) was added to a solution of 3-aminobenzofuran-2-carboxamide (0.25 g; 1.42 mmol) in 1,4-dioxane (1 mL). Then, the product from step 1 (0.84 g; 2.84 mmol) was added, the temperature was increased to 120° C. and stirring was continued until TLC indicated almost completed conversion (here: 18 h). The reaction mixture was diluted with water (15 mL) and extracted with DCM (3×7 mL). The pH of the resulting aq layer was adjusted in between 4 and 5 and the precipitated solid filtered off.

ESI-MS: 353 [M+H]⁺

Rₜ (HPLC): 1.61 min (method H)

Step 3:

The product from step 2 was reacted according to general procedure Int-C.

ESI-MS: 371 [M+H]⁺

Rₜ (HPLC): 1.82 min (method H)

Step 4:

The product from step 2 was reacted according to general procedure Int-D to yield the title compound.

ESI-MS: 522.6 [M+H]⁺

Rₜ (HPLC): 2.96 min (method G)

Intermediate N-M:

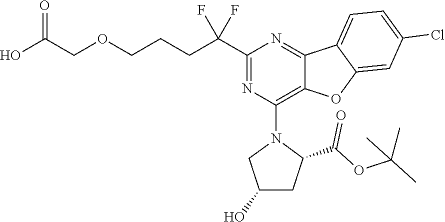 [SMILES: CC(C)(C)OC(=O)[C@@H]1C[C@H](O)CN1c1nc(C(F)(F)CCCOCC(=O)O)nc2c1oc1cc(Cl)ccc12]

Preparation was performed analogously to the procedure described for the synthesis of intermediate N-L applying 3-amino-6-chloro-1-benzofuran-2-carboxamide (prepared as described in EP1710233) as starting material in step 2.

Intermediate N-N:

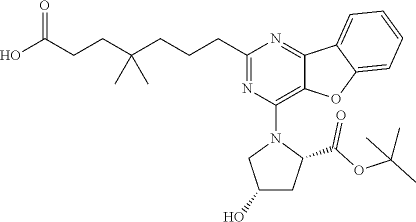 [SMILES: CC(C)(CCCc1nc(N2C[C@@H](O)C[C@H]2C(=O)OC(C)(C)C)c2oc3ccccc3c2n1)CCC(=O)O]

Preparation from intermediate N-R and methyl 4,4-dimethylhept-6-enoate analogously to the reaction sequence described for the synthesis of intermediate N-J.

Intermediate N-O:

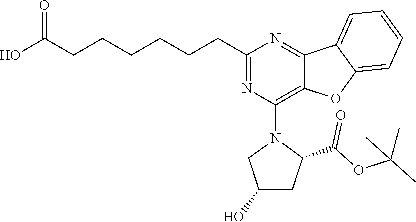 [SMILES: CC(C)(C)OC(=O)[C@@H]1C[C@H](O)CN1c1nc(CCCCCCC(=O)O)nc2c1oc1ccccc12]

Preparation from intermediate N-R and ethyl 6-heptenoate analogously to the reaction sequence described for the synthesis of intermediate N-J.

Intermediate N-P:

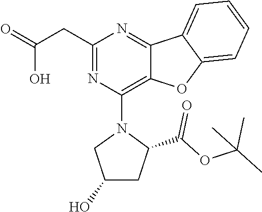 [SMILES: CC(C)(C)OC(=O)[C@@H]1C[C@H](O)CN1c1nc(CC(=O)O)nc2c1oc1ccccc12]

Intermediate N-U was reacted according to general procedure Int-E to yield the title compound.

ESI-MS: 414 [M+H]⁺

Rₜ (HPLC): 0.55 min (method B)

Intermediate N-Q:

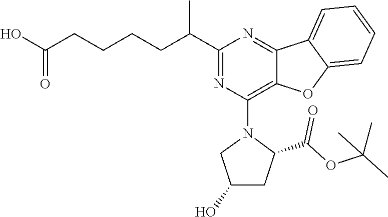 [SMILES: CC(CCCCC(=O)O)c1nc(N2C[C@@H](O)C[C@H]2C(=O)OC(C)(C)C)c2oc3ccccc3c2n1]

tert-butyl (2S,4S)-1-[4-(7-ethoxy-7-oxohept-1-en-2-yl)-8-oxa-3,5-diazatricyclo[7.4.0.0^{2,7}]trideca-1(13),2,4,6,9,11-hexaen-6-yl]-4-hydroxypyrrolidine-2-carboxylate which was formed as a side product in step 1 of the synthesis of intermediate N-O was isolated by RP HPLC and further reacted according to the reaction sequence described for the preparation of intermediate N-O to yield the title compound.

Intermediate N-R:

### tert-Butyl (2S,4S)-1-{4-bromo-8-oxa-3,5-diazatricyclo[7.4.0.0^{2,7}]trideca-1(13),2(7),3,5,9,11-hexaen-6-yl}-4-hydroxypyrrolidine-2-carboxylate

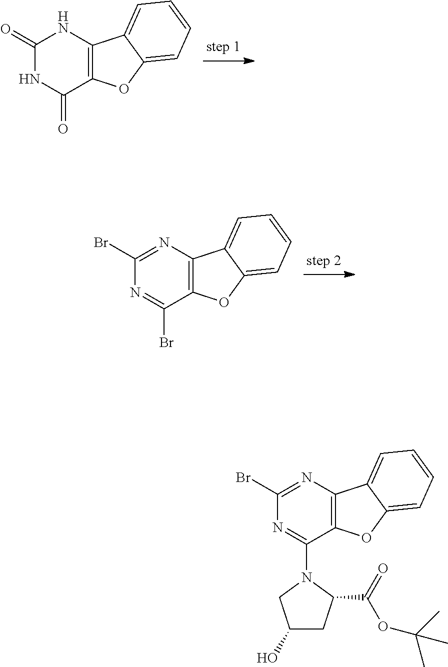 [SMILES: O=c1[nH]c(=O)c2oc3ccccc3c2[nH]1 ; Brc1nc(Br)c2oc3ccccc3c2n1 ; CC(C)(C)OC(=O)[C@@H]1C[C@H](O)CN1c1nc(Br)nc2c1oc1ccccc12]

### Step 1: 4,6-Dibromo-8-oxa-3,5-diazatricyclo[7.4.0.0^{2,7}]trideca-1(13),2(7),3,5,9,11-hexaene

The mixture of 1H-Benzo[4,5]furo[3,2-d]pyrimidine-2,4-dione (11.6 g; 0.0573 mol) and phosphoryl tribromide (40.8 mL, 0.402 mol) was heated at 150° C. for 3 h. The mixture was cooled to RT, the pH was adjusted to pH=7 using aq. Sat. sodium bicarbonate solution while cooling at 0° C., and the mixture was extracted with EtOAc (3×50 mL). The organic layers were combined, dried over sodium sulfate, and evaporated under reduced pressure. The remaining residue was purified by dissolving in a mixture of DCM (3 V to the weight of the crude) and EtOAc (3 V to the weight of the crude) while stirring. After stirring at RT for 30 min, the mixture was filtered and the supernatant was evaporated under reduced pressure.

ESI-MS: 327/329/331 [M+H]⁺

Rₜ (HPLC): 0.67 min (method A)

### Step 2: tert-Butyl (2S,4S)-1-{4-bromo-8-oxa-3,5-diazatricyclo[7.4.0.0^{2,7}]trideca-1(13),2(7),3,5,9,11-hexaen-6-yl}-4-hydroxypyrrolidine-2-carboxylate

To a mixture of 4,6-Dibromo-8-oxa-3,5-diazatricyclo[7.4.0.02,7]trideca-1(13),2(7),3,5,9,11-hexaene (5.56 g, 16.9 mmol) in 93 mL DMF was added tert-butyl (2S,4S)-4-hydroxypyrrolidine-2-carboxylate hydrochloride (4.17 g, 16.6 mmol) and potassium carbonate (7.03 g, 50.8 mmol). After stirring at RT over night, the reaction mixture was poured into water and neutralized with 4M aq. HCl solution. The precipitate was collected by filtration and dried under vacuum.

ESI-MS: 434/436 [M+H]⁺

Rₜ (HPLC): 0.64 min (method A)

Intermediate N-S:

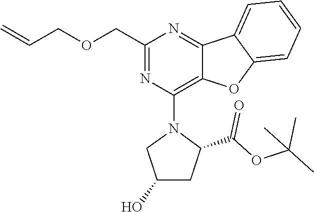 [SMILES: C=CCOCc1nc(N2C[C@@H](O)C[C@H]2C(=O)OC(C)(C)C)c2oc3ccccc3c2n1]

Step 1:

2-Allyloxyacetic acid (9.02 g; 73.8 mmol) was dissolved in DCM with a drop of DMF and cooled to 0° C. Oxalyl chloride (23.4 g; 184 mmol) was added dropwise. The reaction mixture was stirred at 0° C. for 2 h. Then the solvent was evaporated under reduced pressure. The residue was dissolved in DMF and added to a stirred solution of 3-aminobenzofuran-2-carboxamide (13.0 g; 73.8 mmol) in DMF. After 2 h, the mixture was poured into 100 mL of water and stirred for 5 min. The solid formed was collected, dissolved in DCM and dried over magnesium sulfate. Volatiles were evaporated and the solid was triturated with tert-butyl methyl ether to yield 3-[2-(prop-2-en-1-yloxy)acetamido]-1-benzofuran-2-carboxamide.

Step 2:

A suspension of the product from step 1 in 4 M aq. sodium hydroxide was stirred for 2 h at 70° C. The mixture was acidified by addition of aq. hydrochlorid acid and the formed precipitate was collected and dried to afford 4-[(prop-2-en-1-yloxy)methyl]-8-oxa-3,5-diazatricyclo[7.4.0.0^{2,7}]trideca-1(9),2(7),3,10,12-pentaen-6-one.

The product from step 2 was further reacted in a 2 step sequence according to general procedure Int-C and then Int-D to yield the title compound.

ESI-MS: 426 [M+H]⁺

Rₜ (HPLC): 0.49 min (method A)

Intermediate N-T:

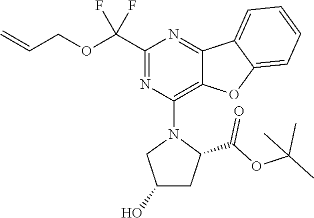 [SMILES: C=CCOC(F)(F)c1nc(N2C[C@@H](O)C[C@H]2C(=O)OC(C)(C)C)c2oc3ccccc3c2n1]

Step 1:

Sodium hydride (60% in mineral oil; 1.73 g, 44.2 mmol) was added portion-wise to sodium 2-chloro-2,2-difluoroacetate (4.5 g, 29.5 mmol) and prop-2-en-1-ol (2.14 g, 36.8 mmol) in THF (30 mL) at 0° C. over a period of 2 minutes under nitrogen. The resulting suspension was stirred at 65° C. for 16 hours. The reaction mixture was cooled and diluted with aq. hydrochloric acid (2 M) until pH=5˜6 was reached and then the aqueous layer was extracted twice with DCM (30 mL). The combined organic layers were washed with brine, dried with sodium sulfate, filtered and evaporated to afford crude product. The crude product was purified by FC (silica gel; petrol ether/EtOAc 0%->30% to afford 2-(allyloxy)-2,2-difluoroacetic acid.

¹H NMR (400 MHz, CDCl₃) δ 9.34 (s, 1H), 6.01-5.91 (m, 1H), 5.45-5.36 (m, 1H), 5.33-5.26 (m, 1H), 4.50 (dt, J=5.8, 1.2 Hz, 2H)

Step 2:

To a solution of the product from step 1 (5 g, 33 mmol) in pyridine (100 mL) was added 3-amino-1-benzofuran-2-carboxamide (4.63 g, 26 mmol), followed by the dropwise addition of phosphoryl trichloride (15.3 g, 0.1 mol) at 0° C. under nitrogen atmosphere. The resulting mixture was stirred overnight at RT. The mixture was diluted with water (200 mL) and extracted three times with EtOAc (200 mL). The combined organic layers were washed with brine, dried over sodium sulfate, and concentrated. The residue was purified by FC (silica gel; petrol ether/EtOAc 15%) to afford 3-(2-(allyloxy)-2,2-difluoroacetamido)benzofuran-2-carboxamide.

ESI-MS: 311 [M+H]⁺

Step 3:

The product from step 2 (3 g, 9.7 mmole) was added into aq. sodium hydroxide (14.5 mL, 4 M, 58.2 mmol), then THF (1.5 mL) was added. The reaction mixture was stirred for 4 h at 70° C. The cooled (RT) reaction mixture was acidified with aq. hydrochloric acid (2 M) to pH=5˜6 and the formed precipitate was collected and dried to afford crude 2-((allyloxy)difluoromethyl)benzofuro[3,2-d]pyrimidin-4(3H)-one which was taken to the next step without further purification.

Step 4:

A 0.5 L 3-neck flask was charged with DMF (1.36 g, 18.6 mmol) and DCM (250 mL) equipped with a thermometer and a nitrogen balloon. The flask was cooled to 0° C., then was added dropwise a solution of oxalyl chloride (3.54 g, 27.9 mmol) in DCM (5 mL) over 5 min, while the temperature was maintained at 0 to 5° C. The mixture was stirred at ambient temperature for 0.5 h. The reaction was cooled in an ice-water bath to 0° C. and the product from step 3 (1.8 g, 6.2 mmol) was added in portions. The reaction was stirred at RT for 15 min, then at 40° C. for 2 h. After cooling to RT, the reaction was poured into ice, neutralized with aq. sodium bicarbonate, extracted twice with DCM (100 mL). The combined organic phase was washed with water, dried with sodium sulfate and concentrated. The crude product was purified by FC (silica gel; petrol ether/EtOAc 2%->10%) to afford the title compound 2-((allyloxy)difluoromethyl)-4-chlorobenzofuro[3,2-d]pyrimidine.

¹H NMR (400 MHz, DMSO-d6) δ 8.41-8.31 (m, 1H), 7.79 (m, 2H), 7.60-7.54 (m, 1H), 7.26 (s, 1H), 6.06 (dq, J=10.8, 6.0 Hz, 1H), 5.45 (dd, J=17.2, 1.2 Hz, 1H), 5.30 (dd, J=10.4, 1.0 Hz, 1H), 4.70 (d, J=6.0 Hz, 2H)

Step 5:

The product from step 4 was reacted according to general procedure Int-D to yield the title compound.

ESI-MS: 462 [M+H]⁺

Rₜ (HPLC): 0.68 min (method A)

Intermediate N-U:

### tert-Butyl (2S,4S)-1-[4-(2-ethoxy-2-oxoethyl)-8-oxa-3,5-diazatricyclo[7.4.0.0^{2,7}]trideca-1(9),2(7),3,5,10,12-hexaen-6-yl]-4-hydroxypyrrolidine-2-carboxylate

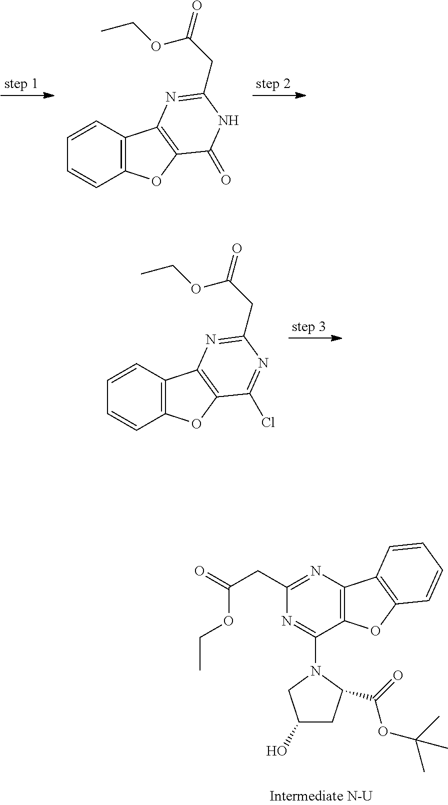 [SMILES: CCOC(=O)Cc1nc2c(oc3ccccc32)c(=O)[nH]1 ; CCOC(=O)Cc1nc(Cl)c2oc3ccccc3c2n1 ; CCOC(=O)Cc1nc(N2C[C@@H](O)C[C@H]2C(=O)OC(C)(C)C)c2oc3ccccc3c2n1]

### Step 1: Ethyl 2-{6-oxo-8-oxa-3,5-diazatricyclo[7.4.0.0^{2,7}]trideca-1(9),2(7),3,10,12-pentaen-4-yl}acetate

To a mixture of ethyl-3-aminobenzofuran-2-carboxylate (5.00 g; 24.4 mmol).) in 4 M aq. hydrochloric acid (50.00 mL; 200 mmol) was added ethyl cyanoacetate (5.19 mL; 48.7 mmol) at RT. The mixture was heated at 100° C. for 4 h. After cooling to RT, another 2.6 mL (24.4 mmol) of ethyl cyanoacetate were added and heating was continued for 48 h at 100° C. The solvent was evaporated and the crude residue was diluted with 100 mL MeOH, filtered and dried. The crude product was taken directly to the next step.

ESI-MS: 273 [M+H]⁺

Rₜ (HPLC): 0.63 min (method B)

### Step 2: Ethyl 2-{6-chloro-8-oxa-3,5-diazatricyclo[7.4.0.0^{2,7}]trideca-1(9),2(7),3,5,10,12-hexaen-4-yl}acetate

A mixture of Ethyl 2-{6-oxo-8-oxa-3,5-diazatricyclo[7.4.0.0^{2,7}]trideca-1(9),2(7),3,10,12-pentaen-4-yl}acetate (3.35 g; 12.3 mmol) in phosphoryl trichloride (50.0 mL; 547 mmol) was heated at 110° C. for 1.5 h. The reaction mixture was cooled to RT and added dropwise to an ice bath (500 mL) under stirring over 30 min. Ethyl acetate was added and the layers were separated. To the organic layer, sat. bicarbonate solution was added slowly, and the phases were separated. The org. layer was washed with water and brine, dried over sodium sulfate, filtered, and concentrated in vacuo.

ESI-MS: 291 [M+H]⁺

Rₜ (HPLC): 0.43 min (method A)

### Step 3: tert-Butyl (2S,4S)-1-[4-(2-ethoxy-2-oxoethyl)-8-oxa-3,5-diazatricyclo[7.4.0.0^{2,7}]trideca-1(9),2(7),3,5,10,12-hexaen-6-yl]-4-hydroxypyrrolidine-2-carboxylate

To Ethyl 2-{6-chloro-8-oxa-3,5-diazatricyclo[7.4.0.0²-7]trideca-1(9),2(7),3,5,10,12-hexaen-4-yl}acetate (8.17 mmol; 1.00 eq., 2.50 g) in 30 mL NMP was added tert-butyl (2S,4S)-4-hydroxypyrrolidine-2-carboxylate hydrochloride (8.99 mmol; 2.01 g) and DIPEA (27.0 mmol; 4.64 mL), and the resulting mixture was stirred at 70° C. for 1.5 h. The reaction mixture was cooled to RT and added slowly to 300 mL of ice-water. The precipitate was filtered, washed several times with water and dried.

ESI-MS: 442 [M+H]⁺

Rₜ (HPLC): 0.67 min (method B)

Intermediate N-V:

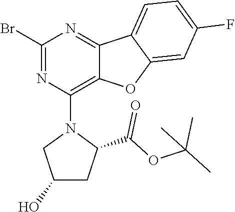 [SMILES: CC(C)(C)OC(=O)[C@@H]1C[C@H](O)CN1c1nc(Br)nc2c1oc1cc(F)ccc12]

Preparation from 11-fluoro-8-oxa-3,5-diazatricyclo[7.4.0.0^{2,7}]trideca-1(13),2(7),9,11-tetraene-4,6-dione analogously to the reaction sequence described for the synthesis of intermediate N-R. The starting material 11-fluoro-8-oxa-3,5-diazatricyclo[7.4.0.0^{2,7}]trideca-1(13),2(7),9,11-tetraene-4,6-dione was prepared analogously to the synthesis of 11-chloro-8-oxa-3,5-diazatricyclo[7.4.0.0^{2,7}]trideca-1(13),2(7),9,11-tetraene-4,6-dione described in WO2019059577.

Preparation of Final Compounds

The absolute configuration of the piperidine moiety of compounds with R2 being a methyl group has in part (e.g. for example 1.01) been assigned from a co-crystal structure with human cGAS protein according to methods described by D. J. Patel et al, PNAS 2019, 11946-11955 (doi.org/10.1073/pnas.1905013116). In other cases, absolute configuration has been assigned based on the assumption, that the more potent diastereoisomer always has (S,S,S,S)-configuration.

### Example 1.01 (General Procedures A, B, C, D)

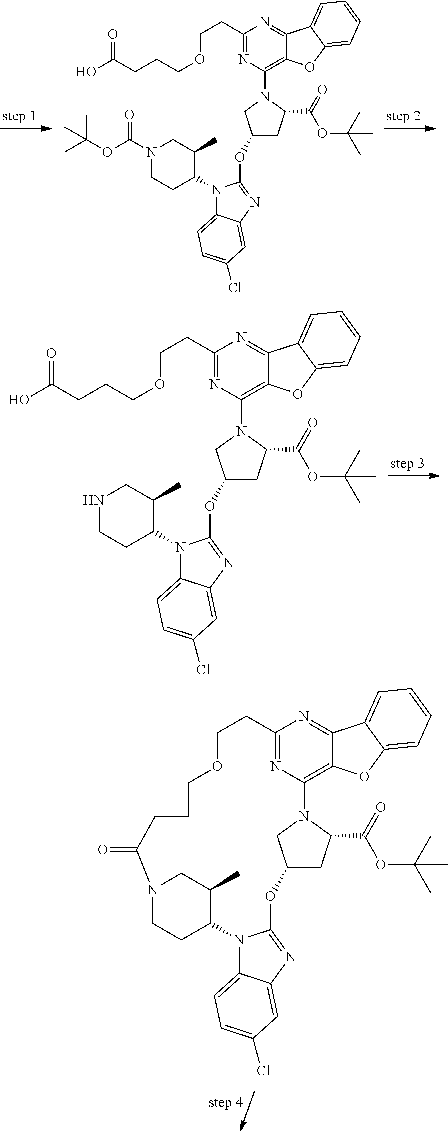

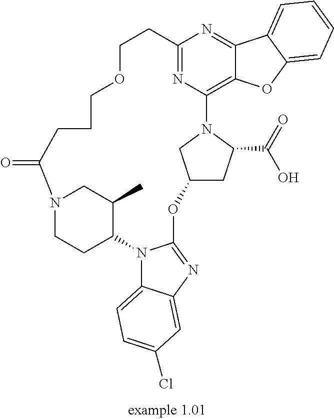

Step 1 (General Procedure A):

In oven-dried glassware under argon atmosphere, to intermediate N-A (216 mg; 0.418 mmol) and intermediate B-01 (188 mg; 0.439 mmol), dissolved in DMA (4.05 ml), was added sodium hydride (55% in mineral oil; 73.0 mg; 1.67 mmol). After bubbling had subsided, the reaction was warmed to 35° C. and stirred for further 25 min. The reaction was then quenched by addition of ice water, diluted with EtOAc, and acidified by addition of aq. hydrochloric acid (1 M; 1.7 mL). The mixture was extracted three times with EtOAc, the combined organic layers were washed subsequently with water and brine, and evaporated to dryness. The crude reaction mixture was purified by FC (silica gel; cyclohexane/EtOAc 25%->100%).

ESI-MS: 833 [M+H]⁺

Rₜ (HPLC): 0.92 min (method B)

Step 2 (General Procedure B):

To a mixture of the intermediate from step 1 (272 mg; 0.326 mmol) and ACN (10 mL) was added TosOH (124 mg; 0.653 mmol) and the mixture was stirred for 3 days at RT in a closed vial. As reaction control by HPLC indicated low conversion, further TosOH (45.0 mg; 0.237 mmol) was added and the mixture was further heated at 40° C. until reaction control by HPLC indicated high conversion to the desired product. DMF and a drop of water were added, the mixture was then filtered and subjected to HPLC purification (Sunfire C18; ACN/water with TFA as modifier).

ESI-MS: 733 [M+H]⁺

Rₜ (HPLC): 0.62 min (method B)

Step 3 (General Procedure C):

In oven-dried glassware under argon, to a solution of HATU (81.2 mg; 0.214 mmol) in DMF (4 mL) was added slowly via a syringe pump a solution of the intermediate from step 2 (145 mg; 0.198 mmol) in DMF (12 mL) under vigorous stirring. The mixture was stirred further until reaction control by HPLC indicated high conversion to the desired product. The reaction was then quenched by addition of water, extracted three times with EtOAc, the combined organic layers were washed subsequently with water and brine, and evaporated to dryness. The crude reaction mixture was purified by FC (cyclohexane/EtOAc 50%->100%, then EtOAc/MeOH 0%->10%).

ESI-MS: 715 [M+H]⁺

Rₜ (HPLC): 0.80 min (method B)

Step 4 (General Procedure D):

A mixture of the intermediate from step 3 (79.0 mg; 0.110 mmol), DCM (1.06 ml) and TFA (852 IL) was stirred at RT for 90 min. As reaction control by HPLC indicated low conversion, temperature was increased to 37° C. and stirring was continued for 3 h. As reaction control by HPLC indicated still low conversion, temperature was increased to 45° C. and stirring was continued until reaction control by HPLC indicated high conversion to the desired product. The mixture was evaporated and the crude product was purified by preparative HPLC (Sunfire C18; ACN/water with TFA as modifier).

ESI-MS: 659 [M+H]⁺

Rₜ (HPLC): 0.83 min (method E)

The following compounds were prepared analogously to example 1.01 described above, following the general procedures A, B, C, and D:

| | | | | Rₜ (HPLC) | |
|---|---|---|---|---|---|
| | Starting | | | [min] | Synthesis |
| Ex. | materials | Structure | ESI-MS | (method) | comment |
| 1.02 | Int N-A + Int. B-02 | 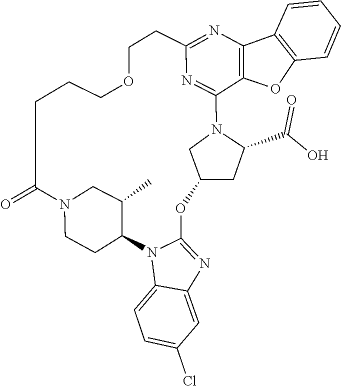 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)CCOCCCC2=O] | 659 [M + H]⁺ | 0.70 (B) | |
| 1.03 | Int N-A + Int. B-03 | 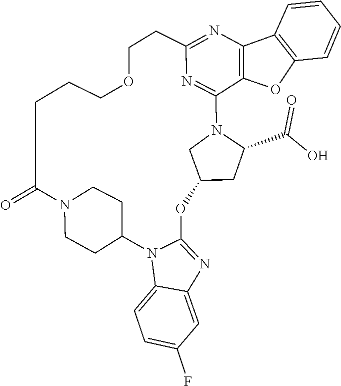 [SMILES: O=C(O)[C@@H]1C[C@H]2CN1c1nc(nc3c1oc1ccccc13)CCOCCCC(=O)N1CCC(CC1)n1c(nc3cc(F)ccc31)O2] | 629 [M + H]⁺ | 0.51 (A) | |
| 1.04 | Int N-A + Int. B-04 | 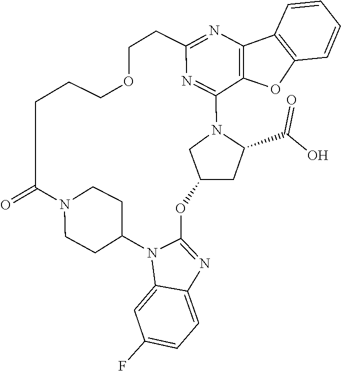 [SMILES: O=C(O)[C@@H]1C[C@H]2CN1c1nc(nc3c1oc1ccccc13)CCOCCCC(=O)N1CCC(CC1)n1c(nc3ccc(F)cc31)O2] | 629 [M + H]⁺ | 0.51 (A) | |
| 1.05 | Int N-A + Int. B-05 | 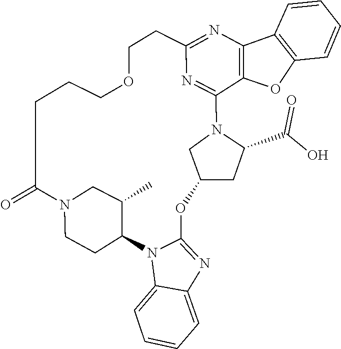 [SMILES: C[C@H]1CN2CC[C@@H]1n1c(nc3ccccc31)O[C@H]1C[C@@H](C(=O)O)N(C1)c1nc(nc3c1oc1ccccc13)CCOCCCC2=O] | 625 [M + H]⁺ | 0.75 (E) | Step 2 applying 4M aq. hydrochloric acid in dioxane at RT |
| 1.06 | Int N-A + Int. B-06 | 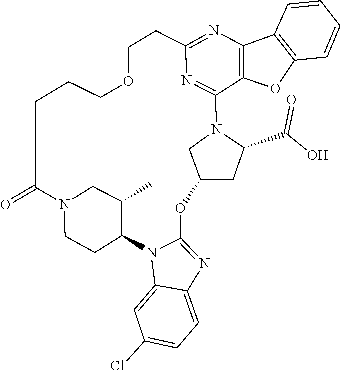 [SMILES: C[C@H]1CN2CC[C@@H]1n1c(nc3ccc(Cl)cc31)O[C@H]1C[C@@H](C(=O)O)N(C1)c1nc(nc3c1oc1ccccc13)CCOCCCC2=O] | 659 [M + H]⁺ | 0.71 (B) | |
| 1.07 | Int N-B + Int. B-05 | 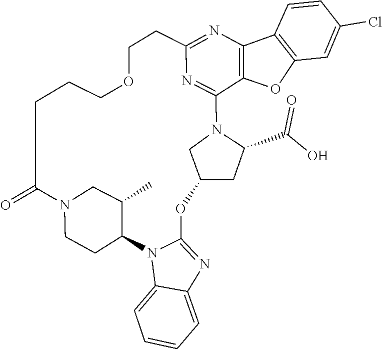 [SMILES: C[C@H]1CN2CC[C@@H]1n1c(nc3ccccc31)O[C@H]1C[C@@H](C(=O)O)N(C1)c1nc(nc3c1oc1cc(Cl)ccc13)CCOCCCC2=O] | 659 [M + H]⁺ | 0.55 (A) | |
| 1.08 | Int N-B + Int. B-02 | 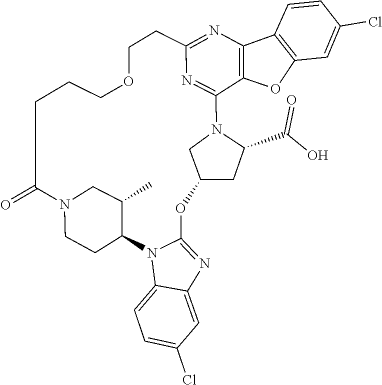 [SMILES: C[C@H]1CN2CC[C@@H]1n1c(nc3cc(Cl)ccc31)O[C@H]1C[C@@H](C(=O)O)N(C1)c1nc(nc3c1oc1cc(Cl)ccc13)CCOCCCC2=O] | 693 [M + H]⁺ | 0.61 (A) | |
| 1.09 | Int N-C + Int. B-02 | 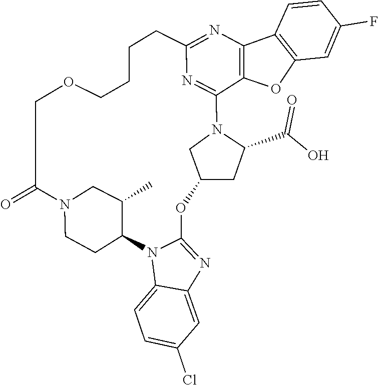 [SMILES: C[C@H]1CN2CC[C@@H]1n1c(nc3cc(Cl)ccc31)O[C@H]1C[C@@H](C(=O)O)N(C1)c1nc(nc3c1oc1cc(F)ccc13)CCCCOCC2=O] | 677 [M + H]⁺ | 0.56 (A) | |
| 1.10 | Int N-D + Int. B-06 | 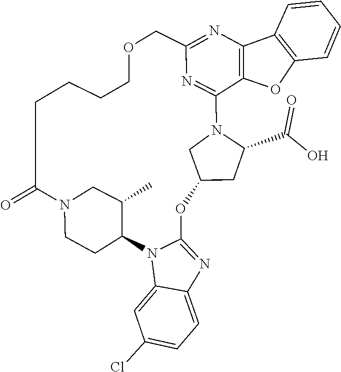 [SMILES: C[C@H]1CN2CC[C@@H]1n1c(nc3ccc(Cl)cc31)O[C@H]1C[C@@H](C(=O)O)N(C1)c1nc(nc3c1oc1ccccc13)COCCCCC2=O] | 659 [M + H]⁺ | 0.59 (A) | |
| 1.11 | Int N-E + Int. B-02 | 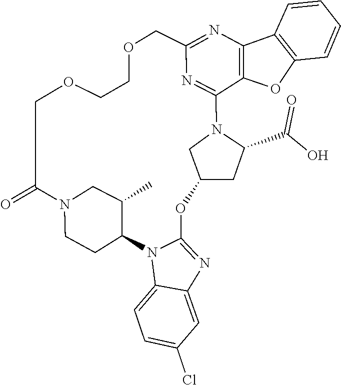 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)COCCOCC2=O] | 661 [M + H]⁺ | 0.82 (C) | Step 3 applying EDC hydrochloride in DCM as coupling reagent |
| 1.12 | Int N-F + Int. B-02 | 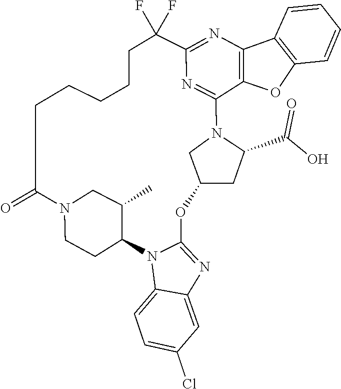 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)CCCCCC2=O] | 693 [M + H]⁺ | 0.79 (A) | |
| 1.13 | Int N-F + Int. B-06 | 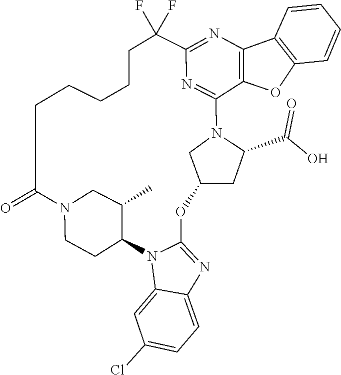 [SMILES: C[C@H]1CN2CC[C@@H]1n1c(nc3ccc(Cl)cc31)O[C@H]1C[C@@H](C(=O)O)N(C1)c1nc(nc3c1oc1ccccc13)C(F)(F)CCCCCC2=O] | 693 [M + H]⁺ | 1.13 (B) | |
| 1.14 | Int N-G + Int. B-05 | 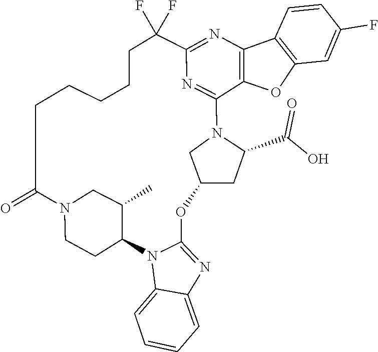 [SMILES: C[C@H]1CN2CC[C@@H]1n1c(nc3ccccc31)O[C@H]1C[C@@H](C(=O)O)N(C1)c1nc(nc3c1oc1cc(F)ccc13)C(F)(F)CCCCCC2=O] | 677 [M + H]⁺ | 0.70 (A) | |
| 1.15 | Int N-G + Int. B-07 | 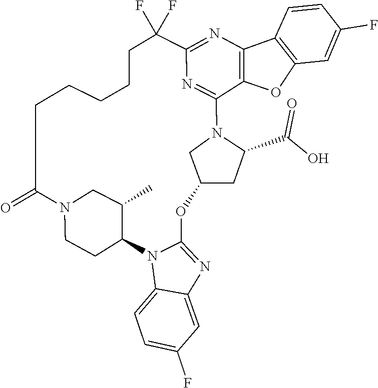 [SMILES: C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O] | 695 [M + H]⁺ | 0.73 (A) | |
| 1.16 | Int N-H + Int. B-05 | 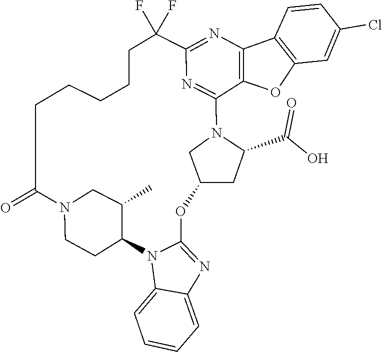 [SMILES: C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O] | 693 [M + H]⁺ | 0.74 (A) | |
| 1.17 | Int N-I + Int. B-05 | 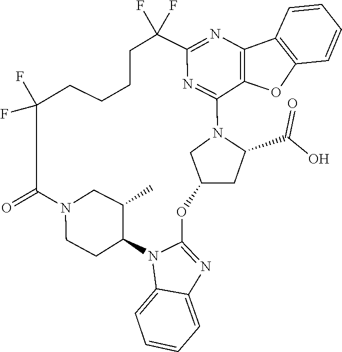 [SMILES: C[C@H]1CN2CC[C@@H]1n1c(nc3ccccc31)O[C@H]1C[C@@H](C(=O)O)N(C1)c1nc(nc3c1oc1ccccc13)C(F)(F)CCCCC(F)(F)C2=O] | 695 [M + H]⁺ | 0.70 (A) | |
| 1.18 | Int N-I + Int. B-07 | 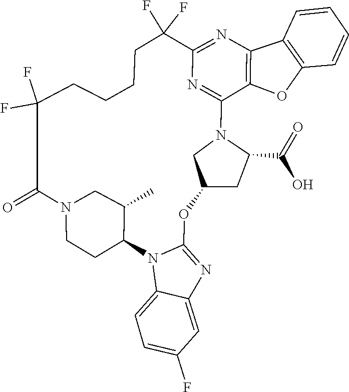 | 713 [M + H]⁺ | 0.73 (A) | |
| 1.19 | Int N-J + Int. B-05 | 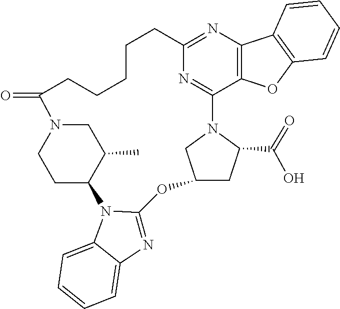 [SMILES: C[C@H]1CN2CC[C@@H]1n1c(nc3ccccc31)O[C@H]1C[C@@H](C(=O)O)N(C1)c1nc(nc3c1oc1ccccc13)CCCCCC2=O] | 609 [M + H]⁺ | 0.59 (B) | |
| 1.20 | Int N-K + Int. B-02 | 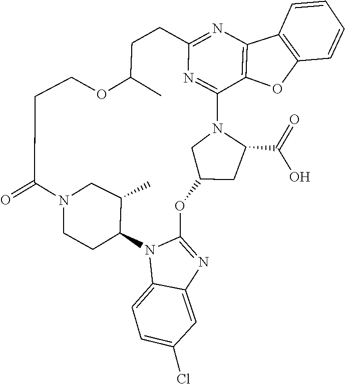 | 673 [M + H]⁺ | 0.55 (A) | |
| 1.21 | Int N-L + Int. B-02 | 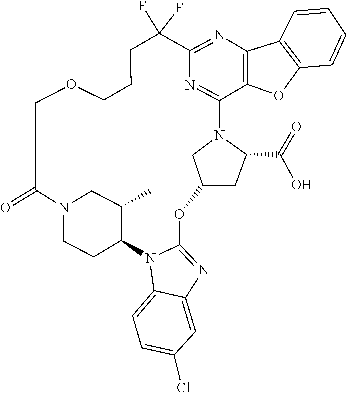 | 695 [M + H]⁺ | 0.69 (A) | |
| 1.22 | Int N-M + Int. B-05 | 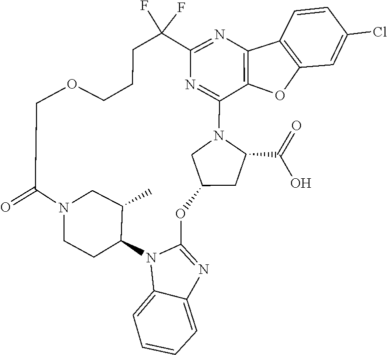 [SMILES: C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCOCC2=O] | 695 [M + H]⁺ | 0.68 (A) | |
| 1.23 | Int N-N + Int. B-05 | 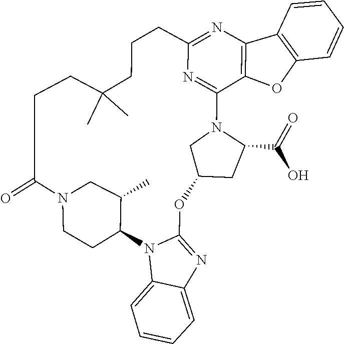 | 651 [M + H]⁺ | 0.59 (A) | |
| 1.24 | Int N-O + Int. B-08 | Mixture of diastereoisomers: racemic trans configuration at piperidine 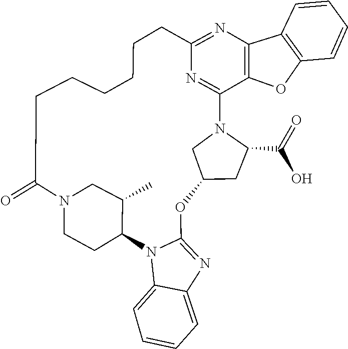 | 623 [M + H]⁺ | 0.84 (E) | |
| 1.25 | Int N-Q + Int. B-10 | 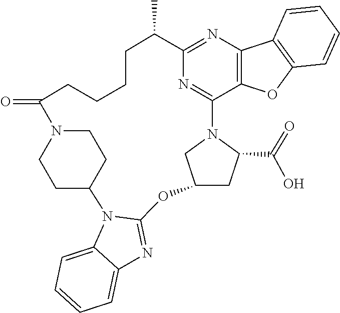 [SMILES: C[C@H]1CCCCC(=O)N2CCC(CC2)n2c(nc3ccccc32)O[C@H]2C[C@@H](C(=O)O)N(C2)c2nc1nc1c2oc2ccccc21] | 609 [M + H]⁺ | 0.60 (B) | Pure diastereomer. Mix of diastereomers resulting from step 3 was separated by RP HPLC (Sunfire C18, ACN/water, modifier: TFA). Diastereomer eluting first was taken to step 4. |
| 1.26 | Int N-Q + Int. B-10 | 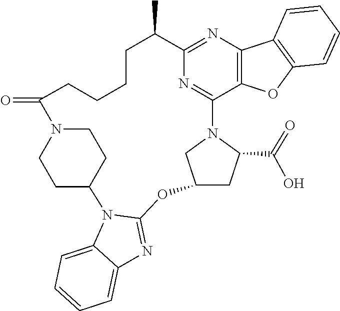 | 609 [M + H]⁺ | 0.60 (B) | Pure diastereomer. Mix of diastereomers resulting from step 3 was separated by RP HPLC (Sunfire C18, ACN/water, modifier: TFA). Diastereomer eluting second was taken to step 4. |
| 1.27 | Int N-F + Int. B-13 | 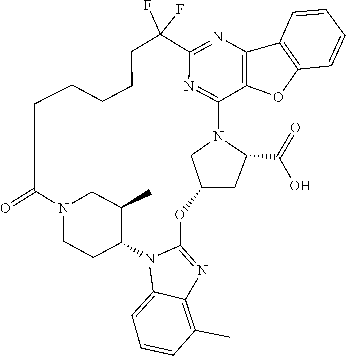 [SMILES: Cc1cccc2c1nc1n2[C@@H]2CCN(C[C@H]2C)C(=O)CCCCCC(F)(F)c2nc(c3oc4ccccc4c3n2)N2C[C@H](C[C@H]2C(=O)O)O1] | 673 [M + H]⁺ | 0.95 (A) | Diastereomer eluting first in purification of final step through preparative RP HPLC (XBridge C18; ACN/H2O; modifier: TFA) |
| 1.28 | Int N-F + Int. B-13 | 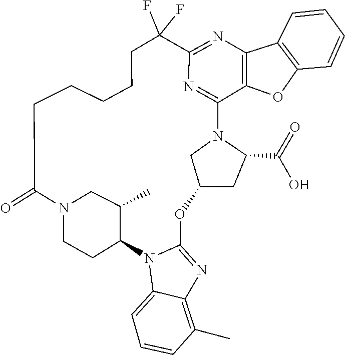 [SMILES: Cc1cccc2c1nc1n2[C@H]2CCN(C[C@@H]2C)C(=O)CCCCCC(F)(F)c2nc(c3oc4ccccc4c3n2)N2C[C@H](C[C@H]2C(=O)O)O1] | 673 [M + H]⁺ | 0.96 (A) | Diastereomer eluting second in purification of final step through preparative RP HPLC (XBridge C18; ACN/H2O; modifier: TFA) |

### Example 2.01

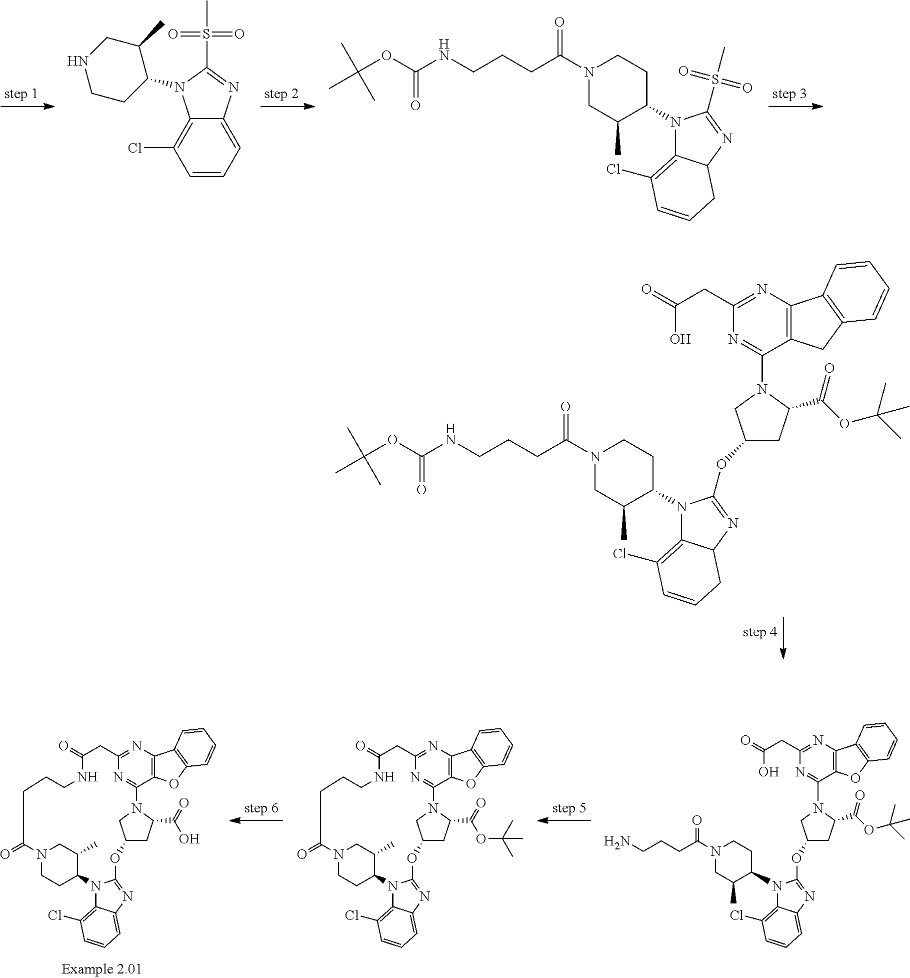

Step 1:

Intermediate B-09 was reacted according to general procedure D to yield the TEA salt of rac-trans-7-chloro-2-methanesulfonyl-1-[3-methylpiperidin-4-yl]-1H-1,3-benzodiazole

Step 2:

The product of step 1 was reacted with 1.2 eq. of 4-{[(tert-butoxy)carbonyl]amino}butanoic acid according to general procedure C.

Step 3:

The product of step 2 was reacted with intermediate N-P according to general procedure A to yield 2-{6-[(2S,4S)-2-[(tert-butoxy)carbonyl]-4-({1-[1-(4-{[(tert-butoxy)carbonyl]amino}butanoyl)-3-methylpiperidin-4-yl]-7-chloro-1H-1,3-benzodiazol-2-yl}oxy)pyrrolidin-1-yl]-8-oxa-3,5-diazatricyclo[7.4.0.0^{2,7}]trideca-1(9),2(7),3,5,10,12-hexaen-4-yl}acetic acid (with racemic trans configuration at the piperidine moiety).

Step 4:

The product of step 3 was BOC-deprotected by reaction with 1.5 eq. hydrochloric acid (4 M in dioxane) in dioxane at RT over night. The crude product was purified by RP HPLC (Sunfire C18; ACN/water/modifier: TFA) to yield a TFA salt of 2-{6-[(2S,4S)-4-({1-[1-(4-aminobutanoyl)-3-methylpiperidin-4-yl]-7-chloro-1H-1,3-benzodiazol-2-yl}oxy)-2-[(tert-butoxy)carbonyl]pyrrolidin-1-yl]-8-oxa-3,5-diazatricyclo[7.4.0.0^{2,7}]trideca-1(9),2(7),3,5,10,12-hexaen-4-yl}acetic acid (with racemic trans configuration at the piperidine moiety).

Step 5:

The product of step 4 was reacted according to general procedure C to yield tert-butyl (12S,14S)-4-chloro-37-methyl-29,34-dioxo-11,18-dioxa-2,9,15,26,30,35,40-heptaazaoctacyclo[33.2.2.1^{12,15}.1^{16,27}.0^{2,10}.0^{3,8}.0^{17,25}.0^{19,24}]hentetraconta-3,5,7,9,16(40),17(25),19(24),20,22,26-decaene-14-carboxylate (with racemic trans configuration at the piperidine moiety).

Step 6:

The product of step 5 was reacted according to general procedure D to yield the title compound (12S,14S)-4-chloro-37-methyl-29,34-dioxo-11,18-dioxa-2,9,15,26,30,35,40-heptaazaoctacyclo[33.2.2.1^{12,15}.1^{16,27}.0^{2,10}.0^{3,8}.0^{17,25}.0^{19,24}]hentetraconta-3,5,7,9,16(40),17(25),19(24),20,22,26-decaene-14-carboxylic acid (with racemic trans configuration at the piperidine moiety).

ESI-MS: 672 [M+H]⁺

Rₜ (HPLC): 0.88 min (method E)

### Example 3.01

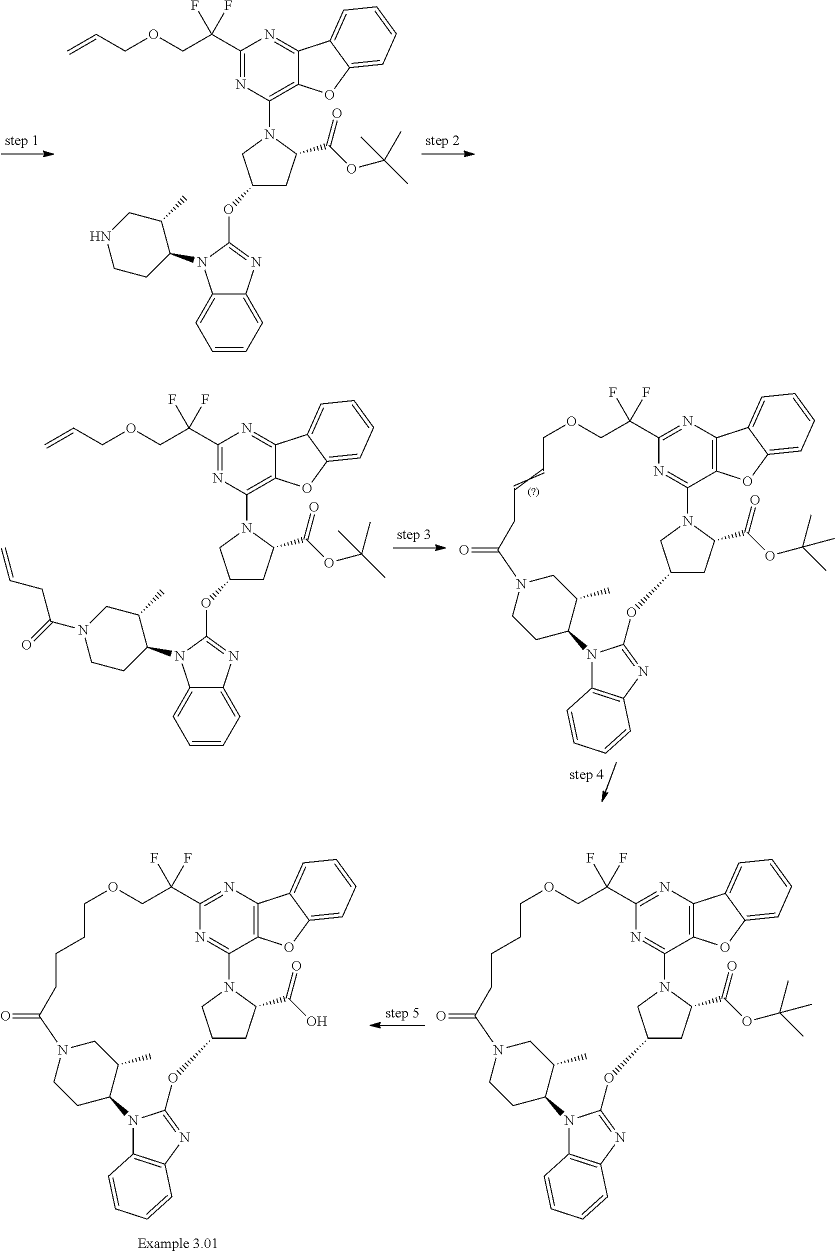

Step 1:

Intermediate 1.02 was BOC deprotected according to general procedure B to yield the TFA salt of tert-butyl (2S,4S)-1-{4-[1,1-difluoro-2-(prop-2-en-1-yloxy)ethyl]-8-oxa-3,5-diazatricyclo[7.4.0.0^{2,7}]trideca-1(13),2,4,6,9,11-hexaen-6-yl}-4-({1-[(3S,4S)-3-methylpiperidin-4-yl]-1H-1,3-benzodiazol-2-yl}oxy)pyrrolidine-2-carboxylate.

ESI-MS: 689 [M+H]⁺

Rₜ (HPLC): 0.62 min (method A)

Step 2:

General procedure G:

To a solution of the product from step 1 (105 mg; 0.127 mmol), vinylacetic acid (14.5 μl; 0.165 mmol) and DMAP (2.0 mg; 0.016 mmol) in DCM (1.05 mL) at 0° C. was added dropwise DCC (1 M; 165 IL; 0.165 mmol). While stirring, the mixture was allowed to warm to RT and then stirred for another hour. The mixture was evaporated to dryness and the residue was subjected to FC (CH/EtOAc 40%->100%). The resulting impure product was further purified by RP HPLC (Sunfire C18; ACN/water, modifier: TFA) to yield tert-butyl (2S,4S)-4-({1-[(3S,4S)-1-(but-3-enoyl)-3-methylpiperidin-4-yl]-1H-1,3-benzodiazol-2-yl}oxy)-1-{4-[1,1-difluoro-2-(prop-2-en-1-yloxy)ethyl]-8-oxa-3,5-diazatricyclo[7.4.0.0^{2,7}]trideca-1(13),2,4,6,9,11-hexaen-6-yl}pyrrolidine-2-carboxylate.

ESI-MS: 757 [M+H]⁺

Rₜ (HPLC): 0.77 min (method A)

Step 3:

General Procedure H:

To a solution of the product from step 2 (dried by azeotropic codistillation with toluene; 77.0 mg; 0.102 mmol) in 1,2-dichloroethane (degassed; 9.0 ml) in an oven-dried glass vessel was added the catalyst Grubbs II (5.0 mg). The mixture was stirred for 24 h at 80° C., then the same amount of catalyst was added again and the mixture was stirred for another 24 h. The reaction was quenched by addition of imidazole (10 mg), then allowed to cool to RT. The mixture was concentrated and subjected to FC (silica gel; CH/EtOAc 20%->100%) to yield tert-butyl (1S,12S,14S,32E,38S)-28,28-difluoro-38-methyl-35-oxo-11,18,30-trioxa-2,9,15,26,36,41-hexaazaoctacyclo[34.2.2.1^{12,15}.1^{16,27}.0^{2,10}.0^{3,8}.0^{17,25}.0^{19,24}]dotetraconta-3(8),4,6,9,16(41),17(25),19,21,23,26,32-undecaene-14-carboxylate.

ESI-MS: 729 [M+H]⁺

Rₜ (HPLC): 0.73 min (method A)

Step 4:

A mixture of the product from step 3 (22.0 mg; 0.0302 mmol), ethanol (1.5 mL) and Pd/C 10% was kept under hydrogen (5 psi) in a Parr apparatus over night. The mixture was filtrated, evaporated and taken to the next step.

ESI-MS: 731 [M+H]⁺

Rₜ (HPLC): 1.09 min (method E)

Step 5:

The product from step 4 (22.1 mg) was reacted according to general procedure D. The product was further purified by RP HPLC (Sunfire C18, ACN/water, modifier: TFA).

ESI-MS: 675 [M+H]⁺

Rₜ (HPLC): 0.60 min (method A)

### Example 4.01

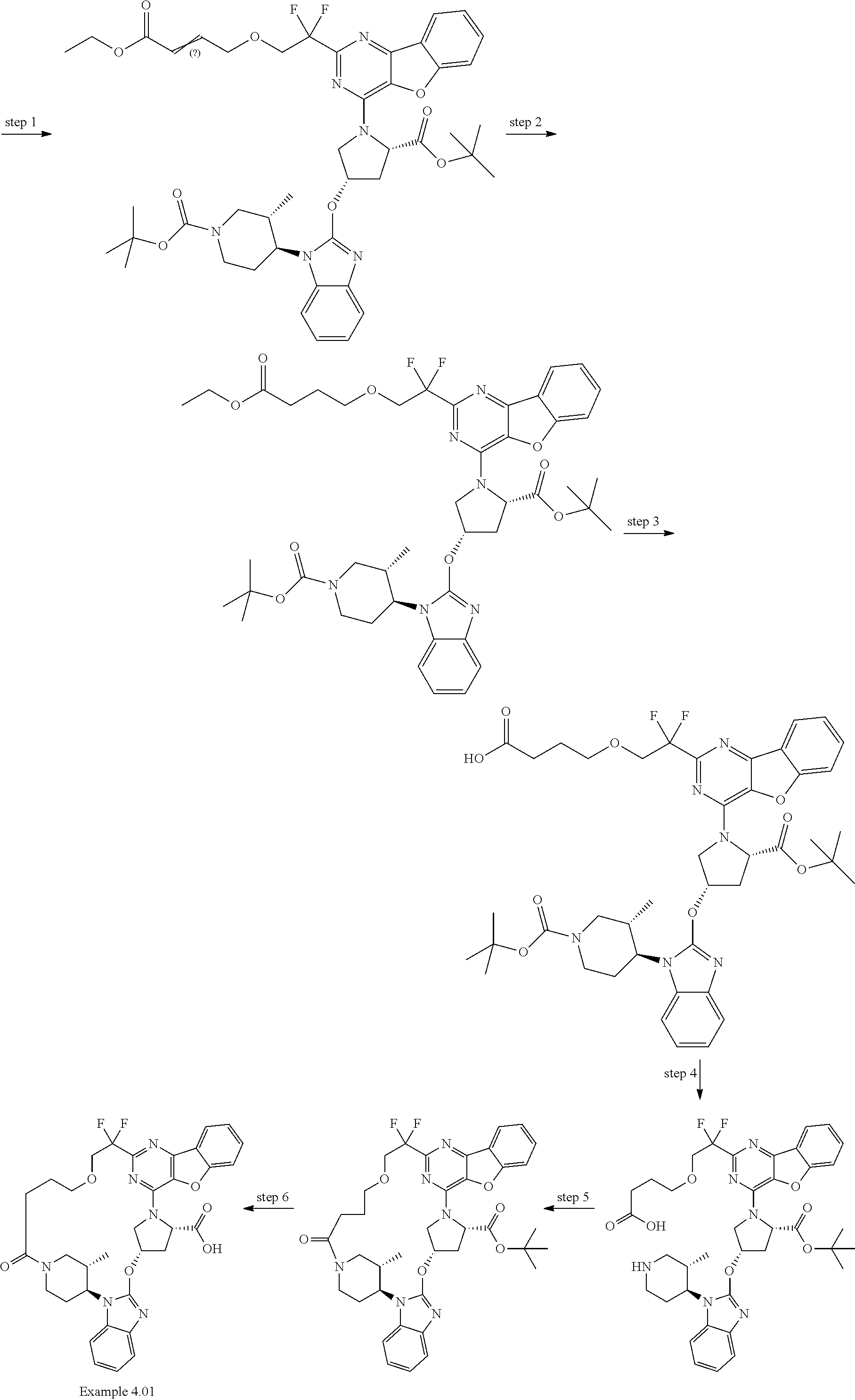

Step 1:

To a degassed solution of intermediate 1.02 (150 mg; 0.190 mmol) in ethyl acrylate (1.56 mL; 14.3 mmol) was added Grubbs II catalyst (8.1 mg; 0.0095 mmol). The mixture was stirred for 24 h, then evaporated and subjected to FC (CH/EtOAc 30%->100%) to yield tert-butyl (3S,4S)-4-(2-{[(3S,5S)-5-[(tert-butoxy)carbonyl]-1-[4-(2-{[4-ethoxy-4-oxobut-2-en-1-yl]oxy}-1,1-difluoroethyl)-8-oxa-3,5-diazatricyclo[7.4.0.0^{2,7}]trideca-1(13),2,4,6,9,11-hexaen-6-yl]pyrrolidin-3-yl]oxy}-1H-1,3-benzodiazol-1-yl)-3-methylpiperidine-1-carboxylate.

ESI-MS: 861 [M+H]⁺

Rₜ (HPLC): 1.28 min (method B)

Step 2:

General Procedure E

A mixture of the product form step 1 (140 mg; 0.163 mmol), Pd/C 10% (28 mg) and ethanol (2.0 mL) was reacted in a Parr apparatus at RT under hydrogen (50 psi) until HPLC control indicated conversion of the starting material (4 hours). The mixture was filtered, concentrated under reduced pressure and subjected to purification by RP HPLC (XBridge C18, ACN/water, modifier: TFA) to yield tert-butyl (3S,4S)-4-(2-{[(3S,5S)-5-[(tert-butoxy)carbonyl]-1-{4-[2-(4-ethoxy-4-oxobutoxy)-1,1-difluoroethyl]-8-oxa-3,5-diazatricyclo[7.4.0.0^{2,7}]trideca-1(13),2,4,6,9,11-hexaen-6-yl}pyrrolidin-3-yl]oxy}-1H-1,3-benzodiazol-1-yl)-3-methylpiperidine-1-carboxylate.

ESI-MS: 863 [M+H]⁺

Rₜ (HPLC): 1.28 min (method B)

Step 3:

General Procedure F

The product from step 2 (118 mg; 0.137 mmol) was taken up in a mixture of aq. Lithium hydroxide (2 M; 153 μL; 0.306 mmol), MeOH (0.80 mL) and THF (4.0 mL). The mixture was stirred at 40° C. until HPLC analytics indicated conversion (7 h), concentrated under reduced pressure, diluted with water and acidified with an equimolar amount of aq. hydrochloric acid. A drop of methanol was added and the mixture was extracted with DCM. The organic layer was separated, concentrated under reduced pressure and taken to the next step.

Step 4:

The product from step 3 was reacted according to general procedure B to yield 4-(2-{6-[(2S,4S)-2-[(tert-butoxy)carbonyl]-4-({1-[(3S,4S)-3-methylpiperidin-4-yl]-1H-1,3-benzodiazol-2-yl}oxy)pyrrolidin-1-yl]-8-oxa-3,5-diazatricyclo[7.4.0.0^{2,7}]trideca-1(13),2,4,6,9,11-hexaen-4-yl}-2,2-difluoroethoxy)butanoic acid.

Step 5:

The product from step 4 was reacted according to general procedure C to yield tert-butyl (1S,12S,14S,37S)-28,28-difluoro-37-methyl-34-oxo-11,18,30-trioxa-2,9,15,26,35,40-hexaazaoctacyclo[33.2.2.1^{12,15}.1^{16,27}.0^{2,10}.0^{3,8}.0^{17,25}.0¹⁹²⁴′]hentetraconta-3,5,7,9,16,19,21,23,25,27(40)-decaene-14-carboxylate.

Step 6:

The product from step 5 was reacted according to general procedure D to yield the title compound

ESI-MS: 661 [M+H]⁺

Rₜ (HPLC): 0.90 min (method B)

### Example 5.01

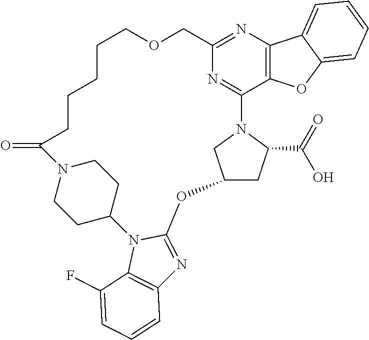 [SMILES: O=C(O)[C@@H]1C[C@H]2CN1c1nc(nc3c1oc1ccccc13)COCCCCCC(=O)N1CCC(CC1)n1c(nc3cccc(F)c31)O2]

Synthesis was performed starting from intermediates N-S and B-11 applying the following reaction sequence:
Step 1: Nucleophilic aromatic substitution applying general procedure A (reagents: N—S and B-11)
Step 2: BOC-deprotection applying general procedure B
Step 3: Reaction with 4-pentenoic acid applying general procedure G
Step 4: Metathesis applying general procedure H
Step 5: Hydrogenation applying general procedure E
Step 6: tert butyl ester deprotection applying general procedure D

ESI-MS: 643 [M+H]⁺

Rₜ (HPLC): 0.58 min (method A)

### Example 6.01

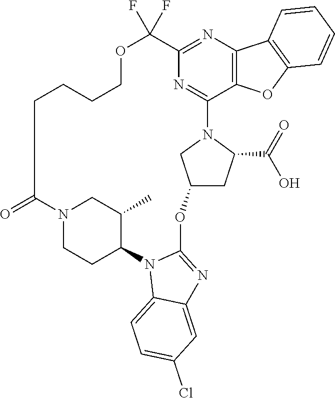 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O]

Synthesis was performed starting from intermediates N-T and B-02 applying the following reaction sequence:
Step 1: Nucleophilic aromatic substitution applying general procedure A (reagents: N-T and B-02)
Step 2: Metathesis with methyl 3-butenoate as second olefin applying a modification of general procedure H (reaction in DCM at RT, applying 15 eq. Of methyl 3-butenoate and after 24 h addition of further 15 eq. of methyl 3-butenoate and further 0.3 eq. of the catalyst in portions.
Step 3: Hydrogenation applying general procedure E (solvent: EtOAc)
Step 4: Ester saponification applying general procedure F (aq. lithium hydroxide/THF, 50° C., 5 h)
Step 5: BOC-deprotection applying general procedure B
Step 6: Ring closing amidation applying general procedure C (solvent: DMA)
Step 7: tert butyl ester deprotection applying general procedure D

ESI-MS: 695 [M+H]⁺

Rₜ (HPLC): 0.72 min (method A)

### Example 7.01

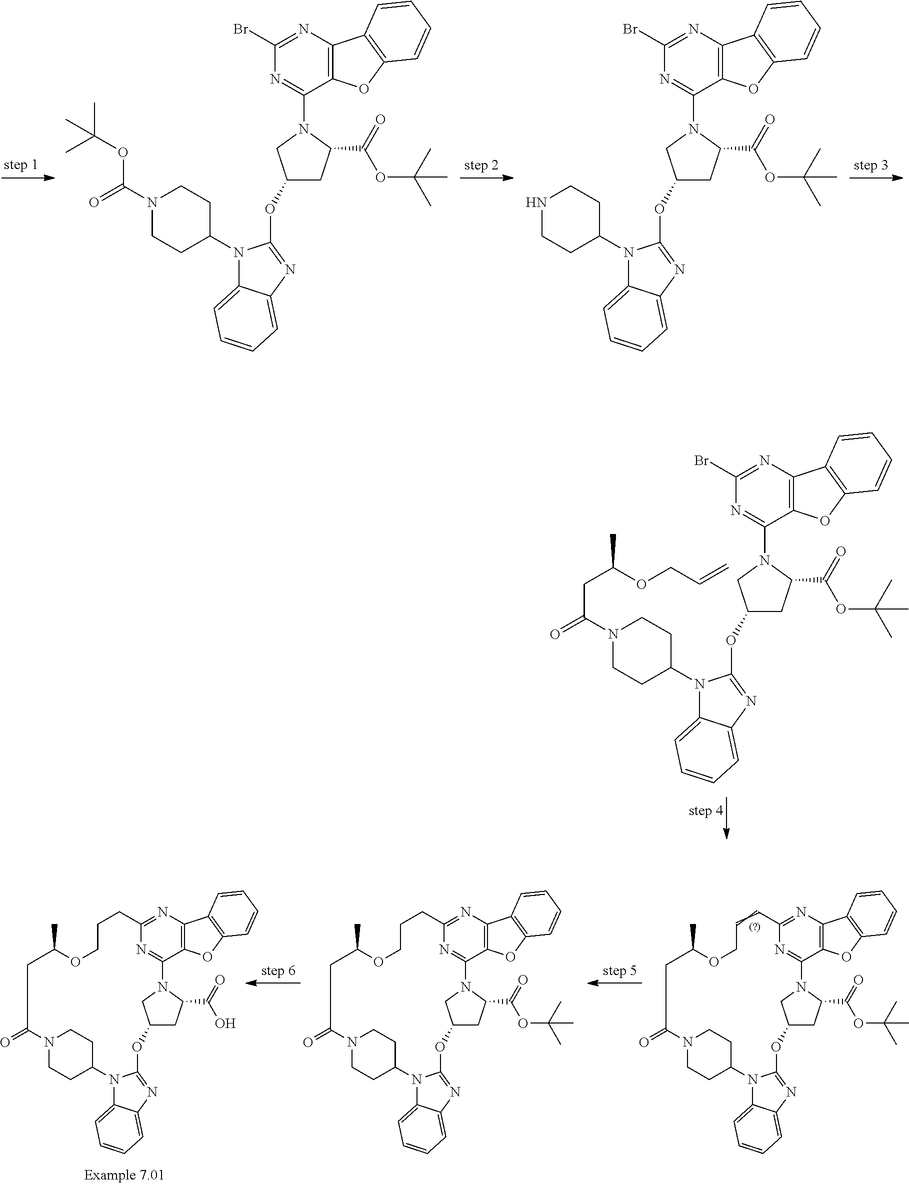

Synthesis was performed starting from intermediates N-R and B-10 applying the following reaction sequence:
Step 1: Nucleophilic aromatic substitution applying general procedure A (reagents: N—R and B-1)
Step 2: BOC-deprotection applying general procedure B
Step 3: Amidation with (3R)-3-prop-3-en-1-yloxy)butanoic acid applying general procedure C

Step 4:

Ta a degassed solution (under argon) of the product from step 3 (162 mg; 0.213 mmol), cesium carbonate (174 mg; 0.533 mmol) and silver iodide (55.1 mg; 0.235 mmol) in dioxane was added the catalyst [1,1′-BIS(DI-TERT-BUTYLPHOSPHINO)FERROCENE]DICHLOROPALLADIUM(II) (21.3 mg; 0.0320 mmol). The mixture was stirred at 120° C. for 24 h, then allowed to cool to RT. The mixture was diluted with ACN/methanol filtered and purified by preparative RP HPLC (XBridge C18, ACN/water, modifier: TFA) to yield tert-butyl (12S,14S,32R)-32-methyl-34-oxo-11,18,31-trioxa-2,9,15,26,35,40-hexaazaoctacyclo[33.2.2.1^{12,15}.1^{16,27}.0^{2,10}.0^{3,8}.0^{17,25}.0^{19,14}]hentetraconta-3(8),4,6,9,16(40),17(25),19(24),20,22,26,28-undecaene-14-carboxylate.

ESI-MS: 679 [M+H]⁺

Rₜ (HPLC): 0.60 min (method A)
Step 5: Hydrogenation applying general procedure E.
Step 6: tert butyl ester deprotection applying general procedure D

ESI-MS: 625 [M+H]⁺

Rₜ (HPLC): 0.47 min (method A)

### Example 8.01

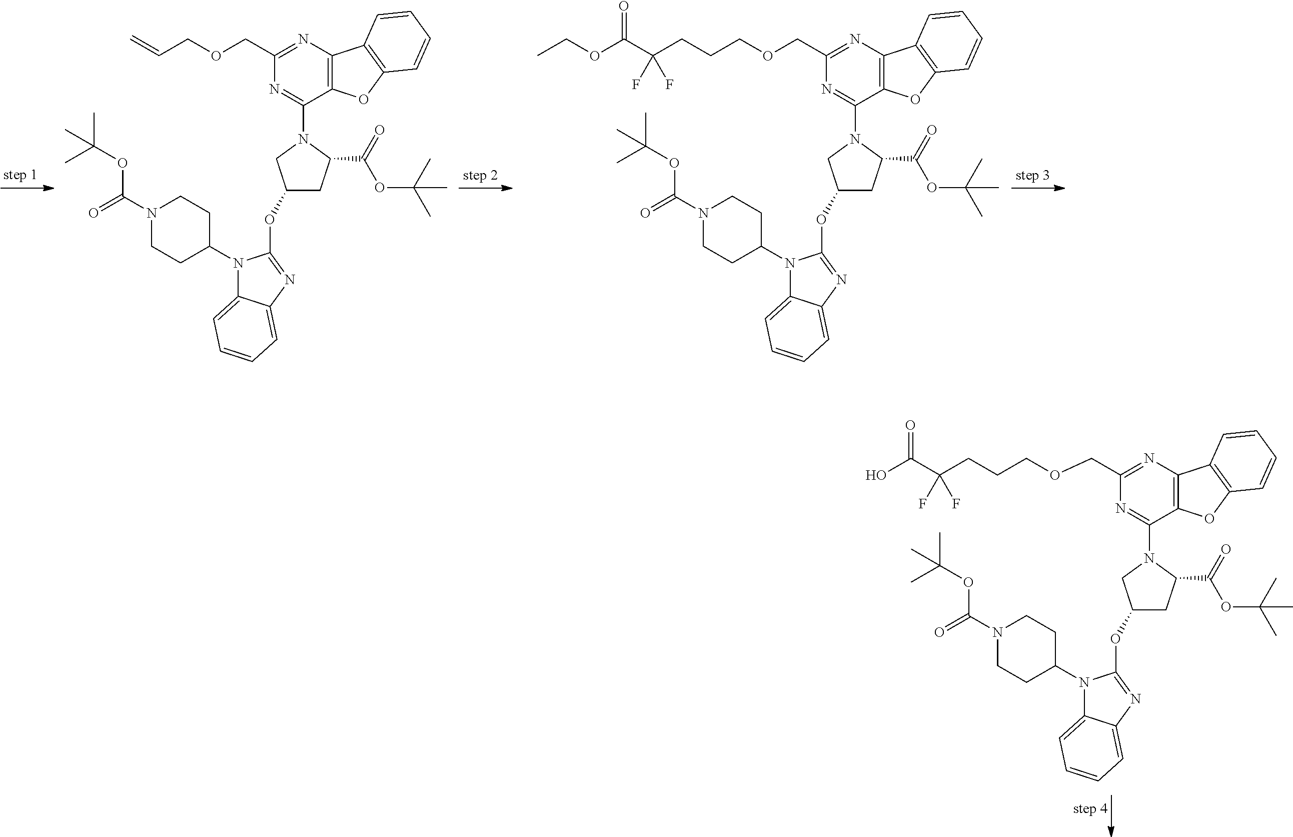 [SMILES: C=CCOCc1nc(N2C[C@@H](Oc3nc4ccccc4n3C3CCN(C(=O)OC(C)(C)C)CC3)C[C@H]2C(=O)OC(C)(C)C)c2oc3ccccc3c2n1 ; CCOC(=O)C(F)(F)CCCOCc1nc(N2C[C@@H](Oc3nc4ccccc4n3C3CCN(C(=O)OC(C)(C)C)CC3)C[C@H]2C(=O)OC(C)(C)C)c2oc3ccccc3c2n1 ; CC(C)(C)OC(=O)[C@@H]1C[C@H](Oc2nc3ccccc3n2C2CCN(C(=O)OC(C)(C)C)CC2)CN1c1nc(COCCCC(F)(F)C(=O)O)nc2c1oc1ccccc12]

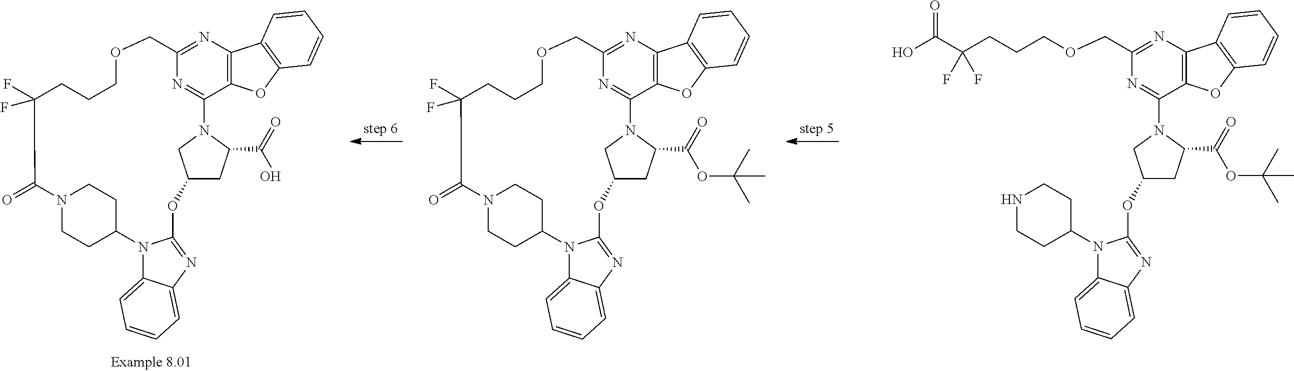 [SMILES: O=C(O)[C@@H]1C[C@H]2CN1c1nc(nc3c1oc1ccccc13)COCCCC(F)(F)C(=O)N1CCC(CC1)n1c(nc3ccccc31)O2 ; CC(C)(C)OC(=O)[C@@H]1C[C@H]2CN1c1nc(nc3c1oc1ccccc13)COCCCC(F)(F)C(=O)N1CCC(CC1)n1c(nc3ccccc31)O2 ; CC(C)(C)OC(=O)[C@@H]1C[C@H](Oc2nc3ccccc3n2C2CCNCC2)CN1c1nc(COCCCC(F)(F)C(=O)O)nc2c1oc1ccccc12]

Synthesis was performed starting from intermediates N-S and B-10 applying the following reaction sequence:

Step 1: Nucleophilic aromatic substitution applying general procedure A (reagents: N—S and B-10)

Step 2:

Reaction under argon. To a degassed mixture of the product from step 1 (381 mg; 0.500 mmol), nickel(II)chloride (3.3 mg; 0.025 mmol), sodium carbonate (53 mg; 0.50 mmol) and anhydrous DMF (4.77 ml) was added at RT ethyl bromodifluoroacetate (209 mg; 1.00 mmol) and phenylsilane (223 mg; 2.00 mmol). The mixture was stirred in a sealed vessel for 14 h at 70° C. the mixture was poured into saturated brine and the resulting mixture was extracted with EtOAc twice. The combined organic layers were dried with sodium sulfate, filtered and evaporated. The crude product was purified by preparative RP HPLC (XBridge C18; ACN/water, modifier: TFA) to yield tert-butyl 4-(2-{[(3S,5S)-5-[(tert-butoxy)carbonyl]-1-(4-{[(5-ethoxy-4,4-difluoro-5-oxopentyl)oxy]methyl}-8-oxa-3,5-diazatricyclo[7.4.0.0^{2,7}]trideca-1(13),2,4,6,9,11-hexaen-6-yl)pyrrolidin-3-yl]oxy}-1H-1,3-benzodiazol-1-yl)piperidine-1-carboxylate.

ESI-MS: 649 [M+H]⁺

Rₜ (HPLC): 0.80 min (method A)
Step 3: Ester saponification applying general procedure F (aq. lithium hydroxide/THF, RT, 2.5 h)
Step 4: BOC-deprotection applying general procedure B
Step 5: Ring closing amidation applying HATU in DMF applying general procedure C
Step 6: tert-butyl ester deprotection applying general procedure D

ESI-MS: 647 [M+H]⁺

Rₜ (HPLC): 0.53 min (method A)

### Example 9.01

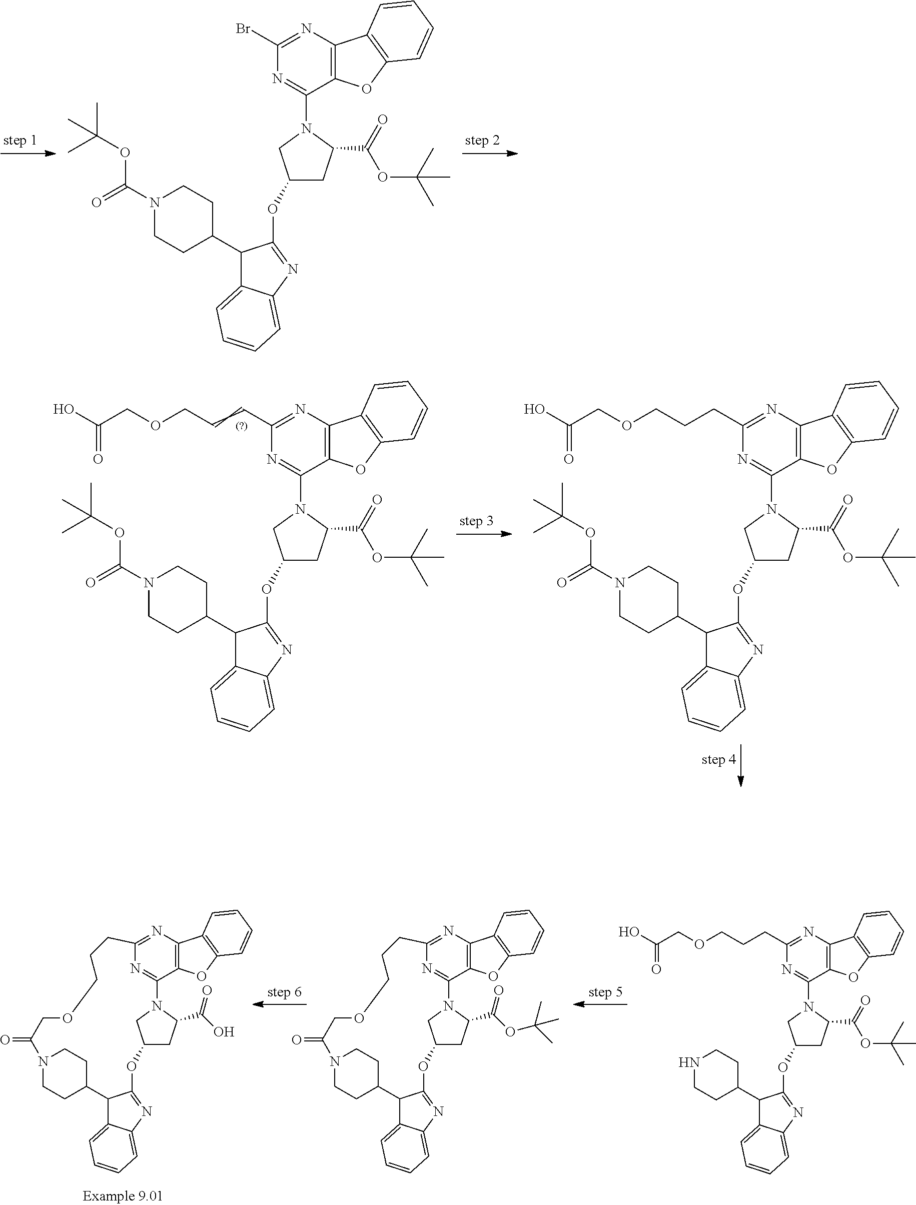

Synthesis was performed starting from intermediates N-R and B-10 applying the following reaction sequence:

Step 1: Nucleophilic aromatic substitution applying general procedure A (reagents: N—R and B-10)

Step 2:

General Procedure I:

Under argon, palladium(II)-acetate (9.0 mg; 0.040 mmol) and Tri-o-tolylphosphine (24.4 mg; 0.080 mmol) were added to a degassed solution of the product from step 1 (326 mg; 0.400 mmol), allyloxy-acetic acid (133 μL; 1.20 mmol) and triethylamine (112 μL; 0.800 mmol) in DMF (6.52 mL). The vial was sealed and the reaction mixture was stirred for 2.5 h at 115° C. The reaction mixture was diluted with ACN/H2O, filtered and purified directly by means of preparative RP HPLC (XBridge C18; 30-100% ACN/H2O, modifier: ammonia) to yield 2-{[(3-{6-[(2S,4S)-2-[(tert-butoxy)carbonyl]-4-[(1-{1-[(tert-butoxy)carbonyl]piperidin-4-yl}-1H-1,3-benzodiazol-2-yl)oxy]pyrrolidin-1-yl]-8-oxa-3,5-diazatricyclo[7.4.0.0^{2,7}]trideca-1(13),2,4,6,9,11-hexaen-4-yl}prop-2-en-1-yl]oxy}acetic acid as a mixture of E/Z isomers.

ESI-MS: 769 [M+H]⁺

Rₜ (HPLC): 0.57/0.58 min (method F)
Step 3: Hydrogenation applying general procedure E (solvent: EtOAc)
Step 4: BOC-deprotection applying general procedure B
Step 5: Ring closing amidation applying HATU in DMF applying general procedure C
Step 6: tert butyl ester deprotection applying general procedure D

ESI-MS: 597 [M+H]⁺

Rₜ (HPLC): 0.42 min (method A)

### Example 10.01 and Example 10.02

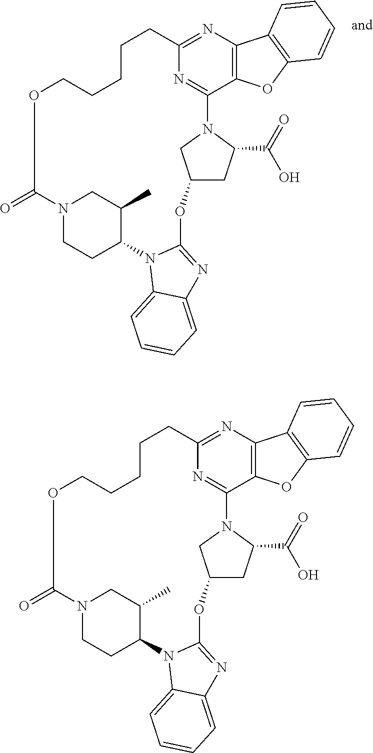 [SMILES: C[C@@H]1CN2CC[C@H]1n1c(nc3ccccc31)O[C@H]1C[C@@H](C(=O)O)N(C1)c1nc(nc3c1oc1ccccc13)CCCCCOC2=O ; C[C@H]1CN2CC[C@@H]1n1c(nc3ccccc31)O[C@H]1C[C@@H](C(=O)O)N(C1)c1nc(nc3c1oc1ccccc13)CCCCCOC2=O]

Synthesis was performed starting from intermediates N-R and 1.03 applying the following reaction sequence:
Step 1: Nucleophilic aromatic substitution applying general procedure A (reagents: N—R and 1.03)
Step 2: Intramolecular Heck coupling applying general procedure I (reaction time 17 h; 100° C.)
Step 3: Hydrogenation applying general procedure E (catalyst: Raney-Ni; solvent: methanol)
Step 4: tert butyl ester deprotection applying general procedure D

Step 5:

The resulting diastereomeric mixture was separated by means of preparative SFC (Instrument: Sepiatec PrepSFC50; Column: CHIRAL ART Cellulose-SC (10 mm*250 mm, 5 um); Mobile phase: A for CO2 and B for IPA [+20 mM NH3]; Gradient: B %=25% isocratic elution mode; Flow rate: 15 mL/min; Wavelength: 220 nm; Column temperature: 40 degrees centigrade; System back pressure: 150 bar).

### Example 10.01 (Eluting First)

ESI-MS: 625 [M+H]⁺

Rₜ (HPLC): 0.804 min (method E)

and example 10.02 (eluting second).

ESI-MS: 625 [M+H]⁺

Rₜ (HPLC): 0.814 min (method E)

### Example 11.01

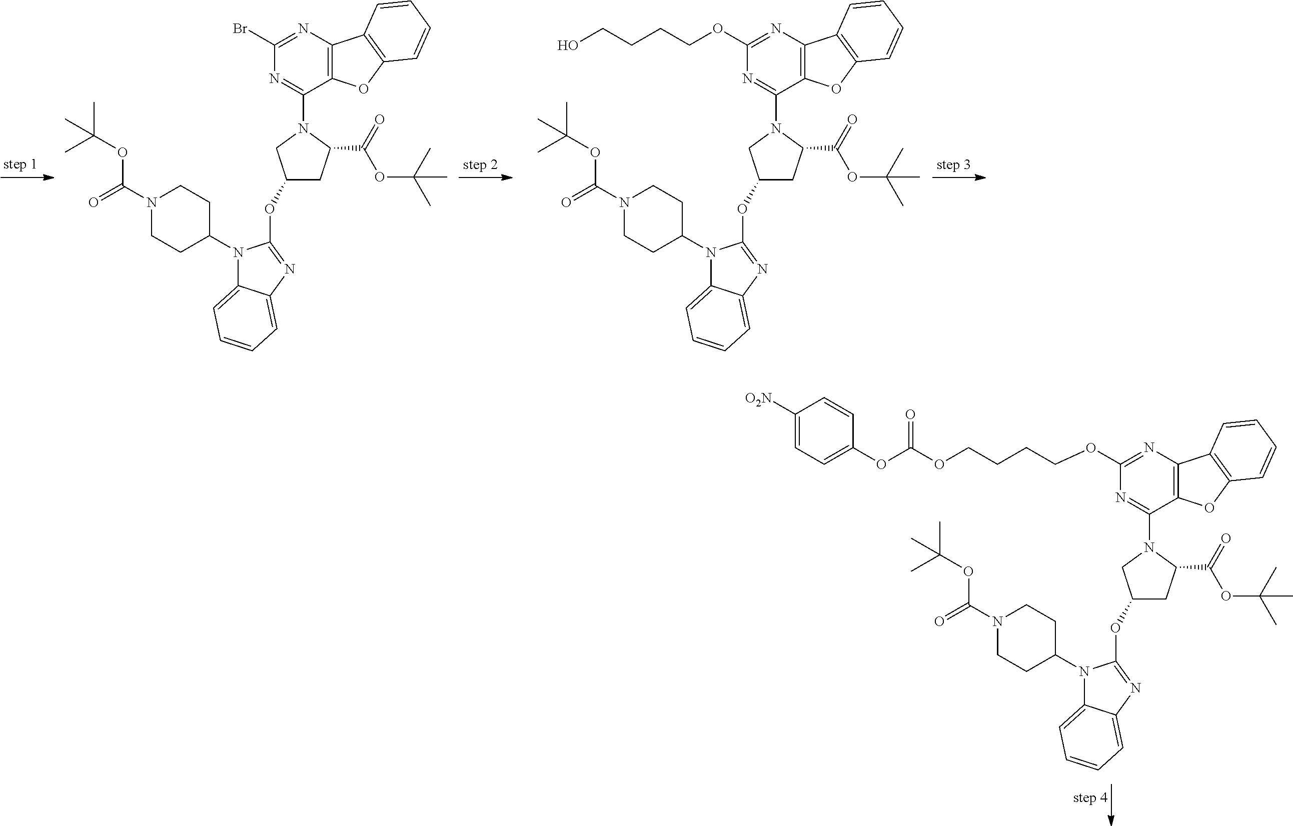 [SMILES: CC(C)(C)OC(=O)[C@@H]1C[C@H](Oc2nc3ccccc3n2C2CCN(C(=O)OC(C)(C)C)CC2)CN1c1nc(Br)nc2c1oc1ccccc12 ; CC(C)(C)OC(=O)[C@@H]1C[C@H](Oc2nc3ccccc3n2C2CCN(C(=O)OC(C)(C)C)CC2)CN1c1nc(OCCCCO)nc2c1oc1ccccc12 ; CC(C)(C)OC(=O)[C@@H]1C[C@H](Oc2nc3ccccc3n2C2CCN(C(=O)OC(C)(C)C)CC2)CN1c1nc(OCCCCOC(=O)Oc2ccc([N+](=O)[O-])cc2)nc2c1oc1ccccc12]

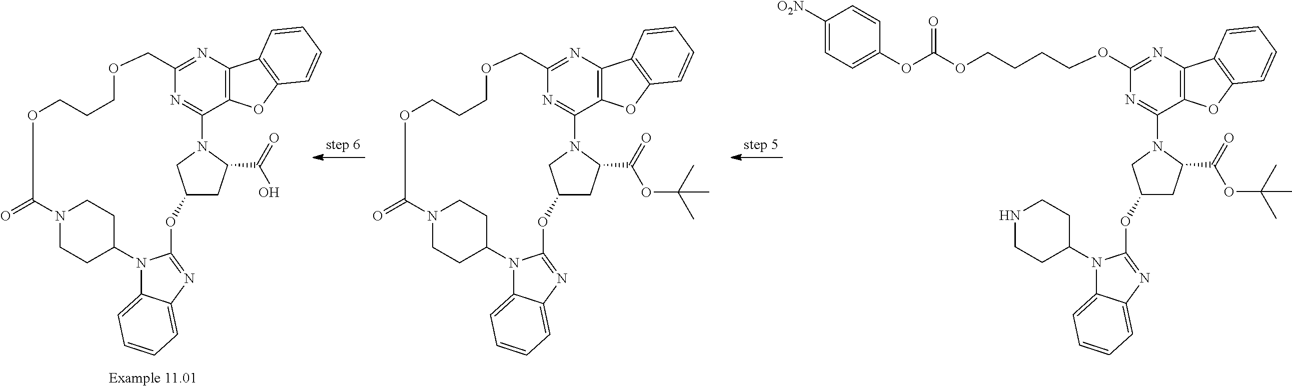 [SMILES: O=C(O)[C@@H]1C[C@H]2CN1c1nc(nc3c1oc1ccccc13)COCCCOC(=O)N1CCC(CC1)n1c(nc3ccccc31)O2 ; CC(C)(C)OC(=O)[C@@H]1C[C@H]2CN1c1nc(nc3c1oc1ccccc13)COCCCOC(=O)N1CCC(CC1)n1c(nc3ccccc31)O2 ; CC(C)(C)OC(=O)[C@@H]1C[C@H](Oc2nc3ccccc3n2C2CCNCC2)CN1c1nc(OCCCCOC(=O)Oc2ccc([N+](=O)[O-])cc2)nc2c1oc1ccccc12]

Synthesis was performed starting from intermediates N-R and B-10 applying the following reaction sequence:

Step 1: Nucleophilic aromatic substitution applying general procedure A (reagents: N—R and B-10)

Step 2:

A mixture of the product from step 1 (500 mg; 0.682 mmol), 1,4-butanediol (1.22 mL; 13.6 mmol), potassium tert-butylate (229 mg; 2.04 mmol) and ACN (10 mL; dried over molecular sieves) was stirred in a sealed vial at 80° C. over night. The mixture was evaporated and the residue was subjected to purification by RP HPLC (Sunfire C18, ACN/water, modifier: TEA) to yield tert-butyl 4-(2-{[(3S,5S)-5-[(tert-butoxy)carbonyl]-1-[4-(4-hydroxybutoxy)-8-oxa-3,5-diazatricyclo[7.4.0.0²′]trideca-1(13),2,4,6,9,11-hexaen-6-yl]pyrrolidin-3-yl]oxy}-1H-1,3-benzodiazol-1-yl)piperidine-1-carboxylate.

ESI-MS: 743 [M+H]⁺

Rₜ (HPLC): 0.70 min (method A)

Step 3:

A mixture of the product from step 2 (143 mg; 0.193 mmol), 4-nitrophenyl chloroformate (87.1 mg; 0.424 mmol), pyridine (103 μL; 1.27 mmol) and DCM (6.0 mL) was stirred at RT for 4 h. Volatiles were evaporated and the crude product was taken to the next step without further purification.

Step 4: Acidic BOC-deprotection was performed applying the conditions of general procedure D.

Step 5:

For carbamate forming ring closure, the crude product from step 4 was taken up in excess DIPEA and stirred for 90 min at 70° C. the mixture was evaporated and taken to the next step without purification.

Step 6: Step 4: tert butyl ester deprotection applying general procedure D

ESI-MS: 613 [M+H]⁺

Rₜ (HPLC): 0.50 min (method A)

### Example 12.01 (Racemic Mixture of Trans Isomers)

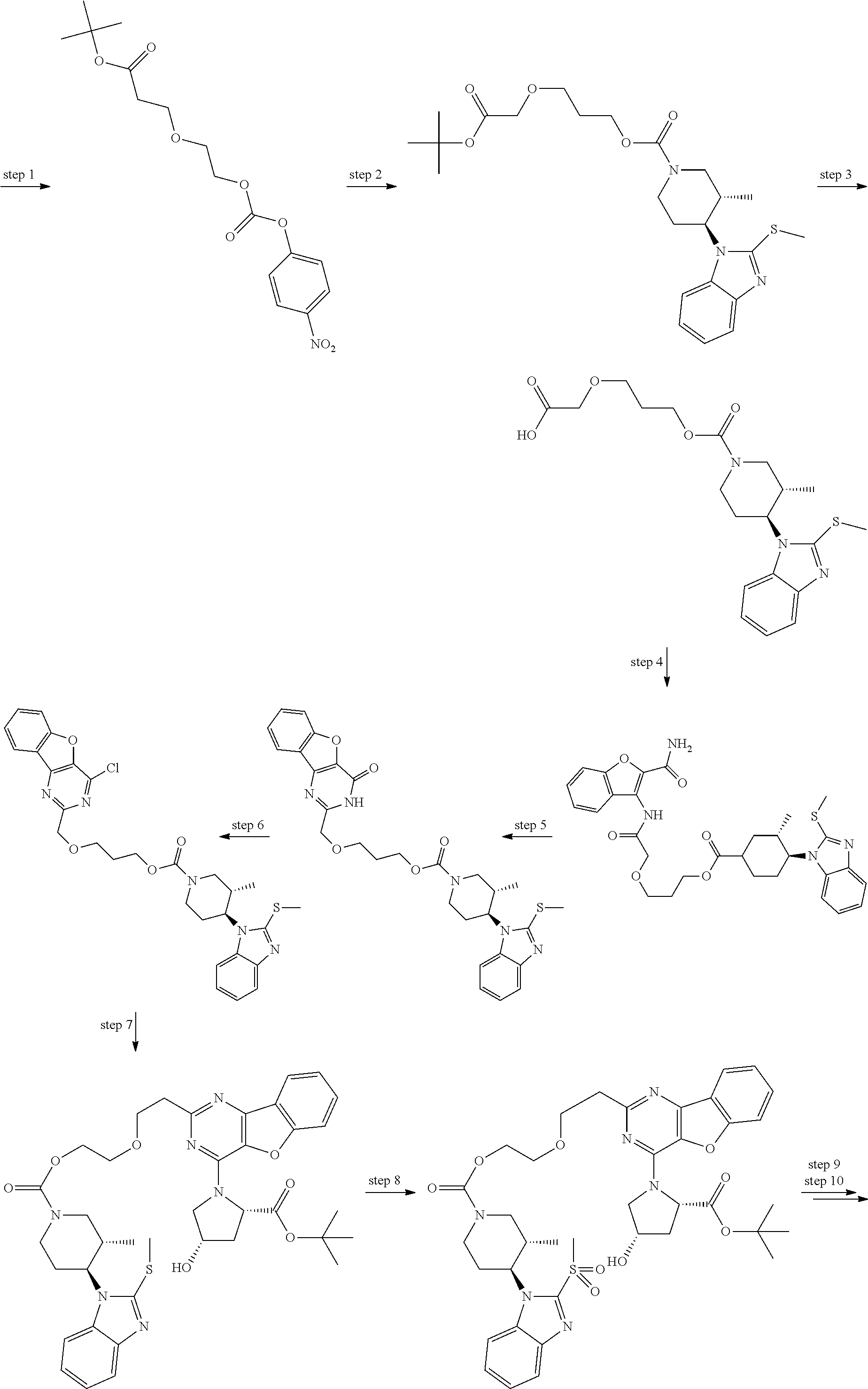

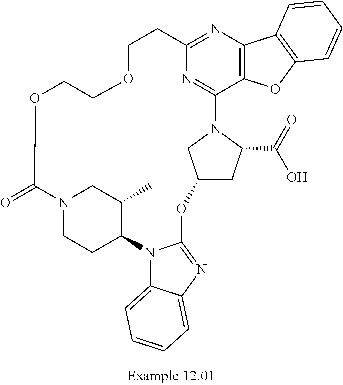 [SMILES: C[C@H]1CN2CC[C@@H]1n1c(nc3ccccc31)O[C@H]1C[C@@H](C(=O)O)N(C1)c1nc(nc3c1oc1ccccc13)CCOCCOC2=O]

Step 1:

To a mixture of tert-butyl-3-(2-hydroxyethoxy)propanoate (1.45 g; 7.62 mmol), pyridine (663 mg 8.38 mmol) and DCM (15 mL) cooled to 0° C., was added dropwise a solution of 4-nitrophenyl-chloroformate (1.54 g; 7.62 mmol) in DCM (15 mL). The mixture was stirred at RT over night, then diluted with DCM and extracted with water. The organic layer was evaporated and the crude product taken to the next step.

Step 2:

A mixture of crude product from step 1 (686 mg), intermediate B-12 (500 mg), diisopropyl-ethylamine (742 μL) and THF (8.0 mL) was refluxed for 2 h. The mixture was evaporated, taken up in EtOAc and extracted twice with aq. sodium hydroxide (1 M), then washed with water and brine. The organic layer was evaporated to dryness and the crude reaction product was purified by FC (silica gel; petrol ether/EtOAc 5%->35%) to yield racemic trans tert-butyl 2-{3-[3-methyl-4-[2-(methylsulfanyl)-1H-1,3-benzodiazol-1-yl]piperidine-1-carbonyloxy]propoxy}acetate.

ESI-MS: 478 [M+H]⁺

Rₜ (HPLC): 0.85 min (method E)

Step 3: Acidic ester cleavage applying general procedure D without chromatographic purification.

Step 4:

To a solution of the product from step 3 (579 mg) in ACN (10 mL) was added 1-chloro-N,N,2-trimethylpropenylamine (259 μL; 3.92 mmol) at RT. The mixture was stirred over night, evaporated, taken up in DCM and extracted with water. The organic layer was evaporated and subjected to FC (silica gel; petrol ether/EtOAc 45%->95%).

ESI-MS: 580 [M+H]⁺

Rₜ (HPLC): 0.81 min (method E) Step 5:

The product from step 4 (650 mg; 1.12 mmol) was suspended in aq. sodium hydroxide (4 M; 15 mL). The mixture was stirred at 60° C. for 1 h, then allowed to cool to RT, acidified by addition of aq. hydrochloric acid (4 M). The supernatant was decanted and the solid was subjected to purification by RP HPLC (Sunfire C18, ACN/water, modifier: TFA).

ESI-MS: 562 [M+H]⁺

Rₜ (HPLC): 0.76 min (method E)

Step 6:

The product from step 5 (390 mg; 0.694 mmol) was suspended in phosphoryl trichloride (6 mL). The mixture was stirred at 90° C. for 1 h, then evaporated. To the residue was added carefully water and the resulting mixture was extracted with EtOAc. The organic layer was separated and evaporated to dryness to yield racemic trans 3-({6-chloro-8-oxa-3,5-diazatricyclo[7.4.0.0^{2,7}]trideca-1(9),2(7),3,5,10,12-hexaen-4-yl}methoxy)propyl-3-methyl-4-[2-(methylsulfanyl)-1H-1,3-benzodiazol-1-yl]piperidine-1-carboxylate.

ESI-MS: 580, 582 [M+H]⁺

Rₜ (HPLC): 0.89 min (method E)

Step 7:

A mixture of the product from step 6 (300 mg; 0.517 mmol), tert-butyl (2S,4S)-4-hydroxypyrrolidine-2-carboxylate hydrochloride (142 mg; 0.621 mmol), potassium carbonate (173 mg; 1.19 mmol) and DMF (7.0 mL) was stirred at RT over night. Water was added and the precipitate was collected and purified by RP HPLC (Sunfire C18, ACN/water, modifier: TFA) to yield 3-({6-[(2S,4S)-2-[(tert-butoxy)carbonyl]-4-hydroxypyrrolidin-1-yl]-8-oxa-3,5-diazatricyclo[7.4.0.0^{2,7}]trideca-1(9),2(7),3,5,10,12-hexaen-4-yl}methoxy)propyl-3-methyl-4-[2-(methylsulfanyl)-1H-1,3-benzodiazol-1-yl]piperidine-1-carboxylate (with racemic trans-configuration of the piperidine moiety).

ESI-MS: 731 [M+H]⁺

Rₜ (HPLC): 0.77 min (method E)

Step 8:

The product from step 7 (239 mg; 0.327 mmol) was dissolved in DCM (8.0 mL). m-Chloroperbenzoic acid (77%; 161 mg; 0.719 mmol) was added and the mixture was stirred for 2 h, then washed with sodium bicarbonate solution. The organic layer was evaporated to yield 3-({6-[(2S,4S)-2-[(tert-butoxy)carbonyl]-4-hydroxypyrrolidin-1-yl]-8-oxa-3,5-diazatricyclo[7.4.0.0^{2,7}]trideca-1(9),2(7),3,5,10,12-hexaen-4-yl}methoxy)propyl-4-(2-methanesulfonyl-1H-1,3-benzodiazol-1-yl)-3-methylpiperidine-1-carboxylate (with racemic trans-configuration of the piperidine moiety).

ESI-MS: 763 [M+H]⁺

Rₜ (HPLC): 0.85 min (method E)

Step 9: Cyclization through aromatic nucleophilic substitution applying general procedure A.

Step 10: Acidic tert butylester cleavage applying general procedure D to yield the title compound.

ESI-MS: 627 [M+H]⁺

Rₜ (HPLC): 0.83 min (method E)

### Example 13.01

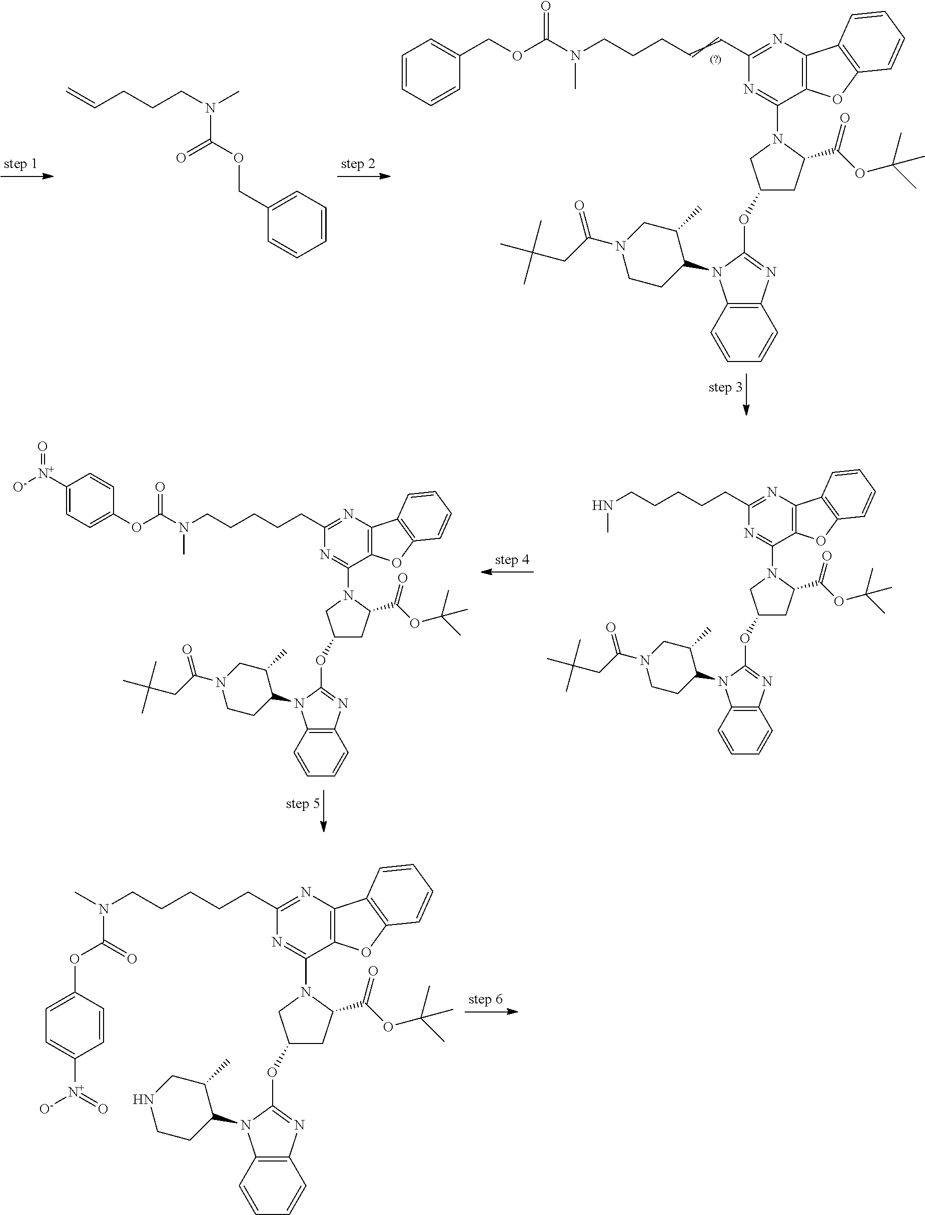

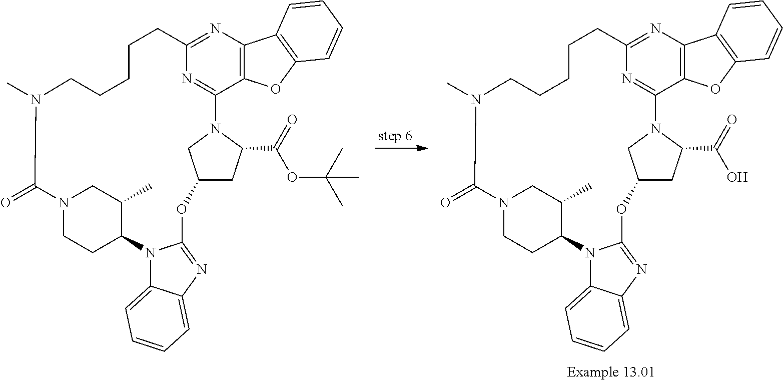 [SMILES: C[C@H]1CN2CC[C@@H]1n1c(nc3ccccc31)O[C@H]1C[C@@H](C(=O)OC(C)(C)C)N(C1)c1nc(nc3c1oc1ccccc13)CCCCCN(C)C2=O ; C[C@H]1CN2CC[C@@H]1n1c(nc3ccccc31)O[C@H]1C[C@@H](C(=O)O)N(C1)c1nc(nc3c1oc1ccccc13)CCCCCN(C)C2=O]

Step 1:

To an ice cold solution of N-methyl-(pent-4-en-1-yl)amine (500 mg; 4.79 mmol) in aq. sodium carbonate (2 M; 4.79 mL; 9.58 mmol) was added dropwise benzyl chloroformate (774 μL; 5.27 mmol). The mixture was stirred for 1 h at 0° C., then diluted with water and extracted three times with EtOAc. The combined organic layers were washed with water and then brine, dried over magnesium sulfated, filtered and concentrated under reduced pressure. The crude product was purified by FC (silica gel; cyclohexane/EtOAc 25%->100%).

ESI-MS: 234 [M+H]⁺

Rₜ (HPLC): 0.67 min (method A)

Step 2:

Reaction under argon in oven-dried glassware. To a mixture of the product from step 1 (191 mg; 0.778 mmol), intermediate 1.01 (200 mg; 0.259 mmol), 1,1′-bis(di-tert-butylphosphino)ferrocene palladium dichloride (20.2 mg; 0.0311 mmol) and previously degassed DMA (2.4 mL) was added triethylamine (180 μL; 1.30 mmol). The reaction mixture was degassed and then heated to 115° C. in a sealed vial for 10 h. The mixture was evaporated and the residue was subjected to purification by preparative RP HPLC (XBridge C18, ACN/water, modifier: ammonia) to yield tert-butyl (3S,4S)-4-(2-{[(3S,5S)-1-{4-[5-{[(benzyloxy)carbonyl](methyl)amino}pent-1-en-1-yl]-8-oxa-3,5-diazatricyclo[7.4.0.0^{2,7}]trideca-1(9),2(7),3,5,10,12-hexaen-6-yl}-5-[(tert-butoxy)carbonyl]pyrrolidin-3-yl]oxy}-1H-1,3-benzodiazol-1-yl)-3-methylpiperidine-1-carboxylate.

Step 3: Hydrogenation (including Cbz-deprotection) applying general procedure E without chromatographic purification.

Step 4:

To a solution of the crude product from step 3 (199 mg) in DCM (3 mL) was added triethylamine (125 μL; 1.04 mmol) and 4-nitrophenyl chloroformate (53 mg; 0.26 mmol). The mixture was stirred at RT for 1 h. The mixture was evaporated and subjected to purification by FC (cyclohexane/EtOAc 20%->100%) to yield tert-butyl (3S,4S)-4-(2-{[(3S,5S)-5-[(tert-butoxy)carbonyl]-1-[4-(5-{methyl[(4-nitrophenoxy)carbonyl]amino}pentyl)-8-oxa-3,5-diazatricyclo[7.4.0.0^{2,7}]trideca-1(9),2(7),3,5,10,12-hexaen-6-yl]pyrrolidin-3-yl]oxy}-1H-1,3-benzodiazol-1-yl)-3-methylpiperidine-1-carboxylate.

Step 5: BOC-deprotection applying general procedure B

Step 6:

The crude product from step 5 was added dropwise via a syringe pump over 25 minutes to a solution of N,N-diisoproyplethylamine (49 μL) in THF (1.5 mL). The mixture was stirred at 80° C. for 10 h. The mixture was passed through a sodium bicarbonate cartridge and evaporated. N-Methylpyrrolidone (3.0 mL) and DIPEA (100 μL) were added and the mixture was heated in a microwave oven to 180° C. for 30 min. The mixture was subjected to purification by RP HPLC (XBridge C18, ACN/water, modifier: TFA).

ESI-MS: 695 [M+H]⁺

Rₜ (HPLC): 0.61 min (method A)

Step 7: tert-Butyl ester cleavage applying general procedure D to yield the title compound.

ESI-MS: 638 [M+H]⁺

Rₜ (HPLC): 0.55 min (method A)

General Technical Remarks

The terms “ambient temperature” and “room temperature” are used interchangeably and designate a temperature of about 20° C., e.g. 15 to 25° C.

As a rule, ¹H NMR spectra and/or mass spectra have been obtained of the compounds prepared. Unless otherwise stated, all chromatographic operations were performed at room temperature.

### List of Abbreviations

ACN acetonitrile
aq. aqueous
BOC tert-butoxycarbonyl
° C. degree Celsius
CH cyclohexane
DCM dichloromethane
DIPEA diisopropylethylamine
DMA dimethylacetamide
DMAP 4-dimethylaminopyridine
DMF N,N-dimethylformamide
DMSO Dimethylsulfoxide
EDC ({[3-(dimethylamino)propyl]imino}methylidene)(ethyl)amine
ESI-MS electrospray ionisation mass spectrometry (m/z)
EtOAc ethyl acetate
FC flash-chromatography, SiO₂ is used if no further details are given
Grubbs II DICHLORO[1,3-BIS(2,4,6-TRIMETHYLPHENYL)-2-IMIDAZOLIDINYLIDENE]-(BENZYLIDENE)-(TRICYCLOHEXYLPHOSPHINE)RUTHENIUM(II)
h hour(s)
HCl hydrogenchloride
HATU [dimethylamino-(1,2,3-triazolo[4,5-b]pyridin-3-yloxy)-methylene]-dimethyl-ammonium hexafluorophosphate
HPLC high performance liquid chromatography
IPA isopropanol
MeOH methanol
min minute
mL milliliter
M molar
n.d. not determined
Pd/C (x %) Palladium (x weight percent) on charcoal
PFTU Pentafluorphenol-tetramethyluronium hexafluorophosphate
rac racemic
RP Reversed phase
RT room temperature (about 20° C.)
SFC Supercritical fluid chromatography
TFA trifluoroacetic acid
THE tetrahydrofurane
TosOH p-toluenesulfonic acid monohydrate
Rₜ retention time in minutes
Vol % volume percent

Analytical Methods (HPLC/SFC):

HPLC Method A:

| Time | Vol % water | Vol % | Flow |
|---|---|---|---|
| [min] | (incl. 0.1% TFA) | ACN | [mL/min] |
| 0.0 | 99.0 | 1.0 | 1.6 |
| 0.02 | 99.0 | 1.0 | 1.6 |
| 1.0 | 0.0 | 100.0 | 1.6 |
| 1.1 | 0.0 | 100.0 | 1.6 |

Column: XBridge BEH C18_2.1×30 mm_1.7 μm (Waters); CT: 60° C.

HPLC Method B:

| Time | Vol % water | Vol % | Flow |
|---|---|---|---|
| [min] | (incl. 0.1% TFA) | ACN | [mL/min] |
| 0.0 | 95.0 | 5.0 | 1.5 |
| 1.3 | 0.0 | 100.0 | 1.5 |
| 1.5 | 0.0 | 100.0 | 1.5 |

Column: Sunfire C18_3.0×30 mm_2.5 μm (Waters); CT: 60° C.

HPLC Method C:

| Time | Vol % water | Vol % | Flow |
|---|---|---|---|
| [min] | (incl. 0.1% TFA) | ACN | [mL/min] |
| 0.0 | 99.0 | 1.0 | 1.5 |
| 0.2 | 99.0 | 1.0 | 1.5 |
| 1.0 | 0.0 | 100.0 | 1.5 |
| 1.1 | 0.0 | 100.0 | 1.5 |

Column: XSelect HSS PFP_2.1×30 mm_1.8 μm (Waters); CT: 60° C.

HPLC Method D:

| Time | Vol % water | Vol % | Flow |
|---|---|---|---|
| [min] | (incl. 0.1% TFA) | ACN | [mL/min] |
| 0.0 | 95.0 | 5 | 2.2 |
| 0.05 | 95.0 | 5 | 2.2 |
| 1.4 | 0.0 | 100.0 | 2.2 |
| 1.8 | 0.0 | 100.0 | 2.2 |

Column: Zorbax StableBond C18_3.0×30 mm_1.8 μm (Agilent); CT: 60° C.

HPLC Method E:

| Time | Vol % water | Vol % | Flow |
|---|---|---|---|
| [min] | (incl. 0.1% TFA) | ACN | [mL/min] |
| 0.0 | 97.0 | 3.0 | 2.2 |
| 0.2 | 97.0 | 3.0 | 2.2 |
| 1.2 | 0.0 | 100.0 | 2.2 |
| 1.25 | 0.0 | 100.0 | 3.0 |
| 1.4 | 0.0 | 100.0 | 3.0 |

Column: Sunfire C18_3.0×30 mm_2.5 μm (Waters); CT: 60° C.

HPLC Method F:

| Time | Vol % water | Vol % | Flow |
|---|---|---|---|
| [min] | (incl. 0.1% TFA) | ACN | [mL/min] |
| 0.0 | 95.0 | 5.0 | 1.3 |
| 0.02 | 95.0 | 5.0 | 1.3 |
| 1.0 | 0.0 | 100.0 | 1.3 |
| 1.3 | 0.0 | 100.0 | 1.3 |

Column: XBridge BEH C18_2.1×30 mm_2.5 urn (Waters); CT: 60° C.

HPLC Method G:

| | Vol % water | Vol % ACN | |
|---|---|---|---|
| Time | (incl. 0.1% | (incl. 0.1% | Flow |
| [min] | formic acid) | formic acid) | [mL/min] |
| 0.0 | 80 | 20 | 1 |
| 3.35 | 20 | 80 | 1 |
| 3.75 | 20 | 80 | 1 |
| 3.9 | 5 | 95 | 1 |
| 4.75 | 5 | 95 | 1 |
| 5.0 | 80 | 20 | 1 |
| 6.0 | 80 | 20 | 1 |

Column: Kinetex XB-C18 2.6 μm (4.6×50 mm), 100 Å; CT: 25° C.

HPLC Method H:

| | Vol % water | Vol % ACN | |
|---|---|---|---|
| Time | (incl. 0.1% | (incl. 0.1% | Flow |
| [min] | formic acid) | formic acid) | [mL/min] |
| 0.0 | 80 | 20 | 0.5 |
| 0.1 | 80 | 20 | 0.5 |
| 1.1 | 0 | 100 | 0.5 |
| 2.5 | 80 | 20 | 0.5 |
| 3.0 | 80 | 20 | 0.5 |

Column: Acquity UPLC BEH C18 1.7 μm (2.1×100 mm); CT: 40° C.

HPLC Method I:

| | Vol % water | Vol % ACN | |
|---|---|---|---|
| Time | (incl. 0.1% | (incl. 0.1% | Flow |
| [min] | formic acid) | formic acid) | [mL/min] |
| 0.0 | 90 | 10 | 1 |
| 3.35 | 30 | 70 | 1 |
| 3.9 | 5 | 95 | 1 |
| 4.75 | 5 | 95 | 1 |
| 5.0 | 90 | 10 | 1 |
| 6.0 | 90 | 10 | 1 |

Column: Kinetex XB-C18 2.6 μm (4.6×50 mm), 100 Å; CT: 25° C.

### 4. EXAMPLES

5.1 Example Compounds of Formula I, II or III of the Invention

The following Example compounds of formula I, II or III as summarized in Table 1 have been synthesized and tested with respect to their pharmacological properties regarding their potency to inhibit cGAS activity.

In particular the “biochemical (in vitro) IC₅₀-values” with regard to cGAS-inhibition (hcGAS IC₅₀), the “IC₅₀-value with regard to the inhibition of IFN induction in virus-stimulated THP1 cells” (THP₍ᵥᵢᵣ₎ IC₅₀), the “IC₅₀-value with regard to the inhibition of IFN induction in cGAMP-stimulated THP1 cells” (THP_{(cGAMP)} IC₅₀) and the “IC₅₀-value with regard to inhibition of IFN induction in dsDNA-stimulated human whole blood” (hWB IC₅₀) has been experimentally determined according to the assay methods as described in section 6 below. The results are summarized in Table 1.

The Example compounds of formula I, II or III as summarized in Table 1 show at the same time the following three properties:
a satisfying “biochemical (in vitro) IC₅₀-value with regard to cGAS inhibition” (with a hcGAS IC₅₀ of ≤100 nM, preferably of 50 nM, in particular of ≤10 nM),
a satisfying “cellular IC₅₀-value regarding cGAS inhibition” (with a THP1₍ᵥᵢᵣ₎ IC₅₀ of ≤1 μM, preferably of ≤500 nM, more preferably of ≤100 nM, in particular of ≤50 nM) and
a satisfying selectivity for cGAS-inhibition
   (with a ratio THP1_{(cGAMP)}IC₅₀/THP1₍ᵥᵢᵣ₎IC₅₀ of ≥10, more preferably ≥50, more preferably ≥500, in particular ≥1000).

Additionally, the Example compounds of formula I, II or III also show acceptable IC₅₀-values with regard to inhibition of IFN induction in dsDNA-stimulated human whole blood (hWB IC₅₀).

TABLE 1
Pharmacological properties of the Example compounds of formula I, II or III of the
invention

| | | | | | | Ratio |
|---|---|---|---|---|---|---|
| | | | | | | THP_{(cGAMP)} |
| Example | | hCGAS | THP₍ᵥᵢᵣ₎ | THP_{(cGAMP)} | hWB | IC₅₀/THP₍ᵥᵢᵣ₎ |
| No. | Structure | IC₅₀ [nM] | IC₅₀ [nM] | IC₅₀ [nM] | IC₅₀ [nM] | IC₅₀ |
| 1.01 | 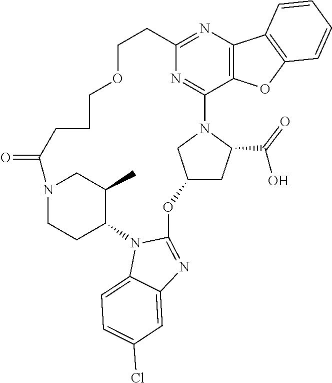 [SMILES: C[C@@H]1CN2CC[C@H]1n1c(nc3cc(Cl)ccc31)O[C@H]1C[C@@H](C(=O)O)N(C1)c1nc(nc3c1oc1ccccc13)CCOCCCC2=O] | 14 | 39 | >10000 | 87 | >253 |
| 1.02 | 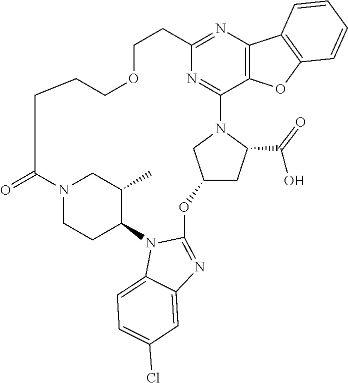 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)CCOCCCC2=O] | 2.8 | 2.8 | >10000 | 5 | >3557 |
| 1.03 | 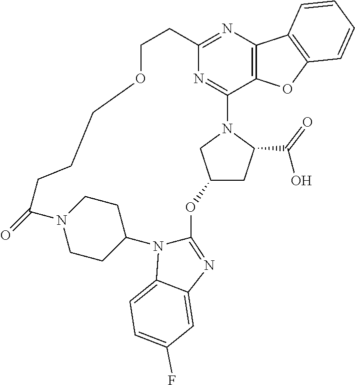 [SMILES: O=C(O)[C@@H]1C[C@H]2CN1c1nc(nc3c1oc1ccccc13)CCOCCCC(=O)N1CCC(CC1)n1c(nc3cc(F)ccc31)O2] | 5.6 | 110 | >10000 | 36 | >90 |
| 1.04 | 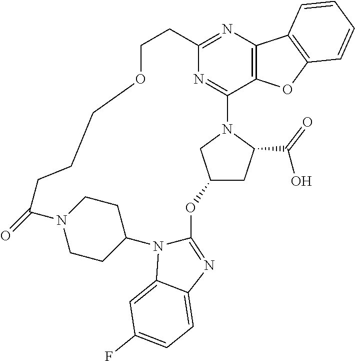 [SMILES: O=C(O)[C@@H]1C[C@H]2CN1c1nc(nc3c1oc1ccccc13)CCOCCCC(=O)N1CCC(CC1)n1c(nc3ccc(F)cc31)O2] | 4.5 | 44 | >10000 | 44 | >229 |
| 1.05 | 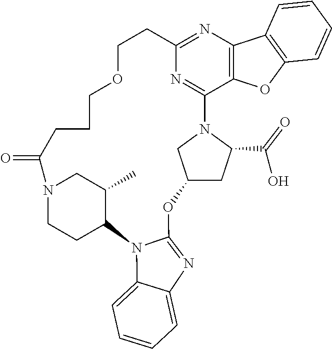 | 1.8 | 2.9 | >10000 | 5 | >3448 |
| 1.06 | 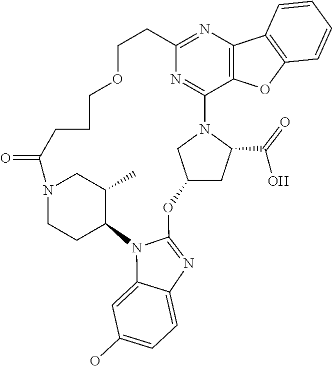 [SMILES: C[C@H]1CN2CC[C@@H]1n1c(nc3ccc(O)cc31)O[C@H]1C[C@@H](C(=O)O)N(C1)c1nc(nc3c1oc1ccccc13)CCOCCCC2=O] | 3.2 | 4.1 | >10000 | 15 | >2417 |
| 1.07 | 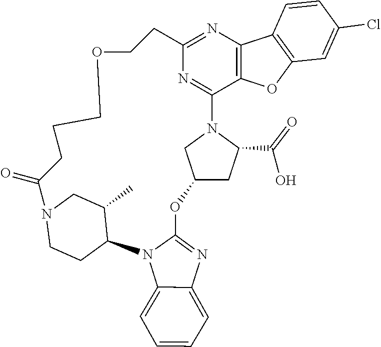 [SMILES: C[C@H]1CN2CC[C@@H]1n1c(nc3ccccc31)O[C@H]1C[C@@H](C(=O)O)N(C1)c1nc(nc3c1oc1cc(Cl)ccc13)CCOCCCC2=O] | 3.8 | 6.5 | >10000 | 13 | >1541 |
| 1.08 | 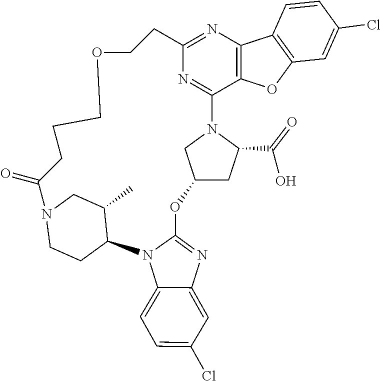 [SMILES: C[C@H]1CN2CC[C@@H]1n1c(nc3cc(Cl)ccc31)O[C@H]1C[C@@H](C(=O)O)N(C1)c1nc(nc3c1oc1cc(Cl)ccc13)CCOCCCC2=O] | 4.5 | 2.4 | 9500 | 17 | 3928 |
| 1.09 | 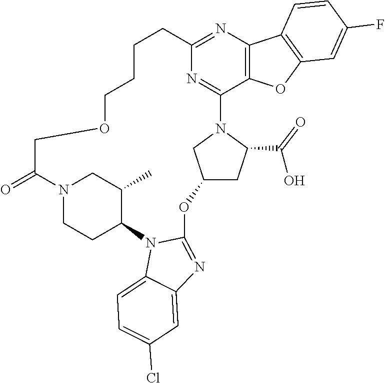 [SMILES: C[C@H]1CN2CC[C@@H]1n1c(nc3cc(Cl)ccc31)O[C@H]1C[C@@H](C(=O)O)N(C1)c1nc(nc3c1oc1cc(F)ccc13)CCCCOCC2=O] | 3.9 | 4.3 | >10000 | 2 | 4305 |
| 1.10 | 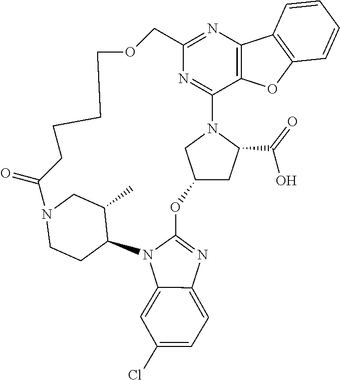 [SMILES: C[C@H]1CN2CC[C@@H]1n1c(nc3ccc(Cl)cc31)O[C@H]1C[C@@H](C(=O)O)N(C1)c1nc(nc3c1oc1ccccc13)COCCCCC2=O] | 5 | 13 | >10000 | 12 | >790 |
| 1.11 | 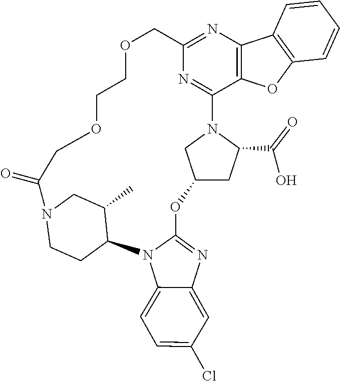 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)COCCOCC2=O] | 1.9 | 3.6 | 10000 | 3 | >2907 |
| 1.12 | 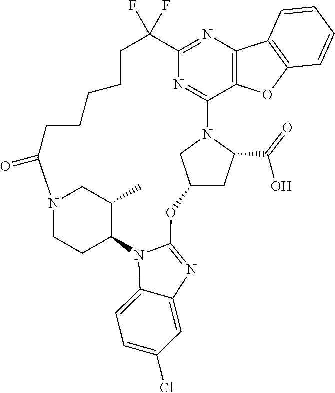 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)CCCCCC2=O] | 5.2 | 2.2 | 3600 | 5 | 1620 |
| 1.13 | 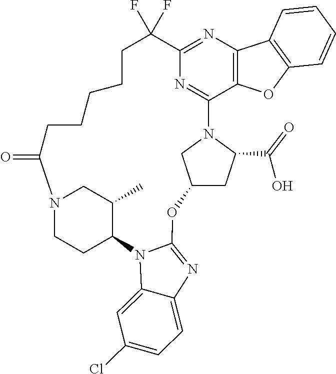 [SMILES: C[C@H]1CN2CC[C@@H]1n1c(nc3ccc(Cl)cc31)O[C@H]1C[C@@H](C(=O)O)N(C1)c1nc(nc3c1oc1ccccc13)C(F)(F)CCCCCC2=O] | 6.1 | 4.9 | 11000 | 15 | 2251 |
| 1.14 | 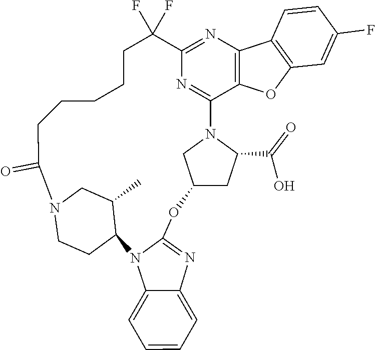 [SMILES: C[C@H]1CN2CC[C@@H]1n1c(nc3ccccc31)O[C@H]1C[C@@H](C(=O)O)N(C1)c1nc(nc3c1oc1cc(F)ccc13)C(F)(F)CCCCCC2=O] | 4.1 | 1.5 | 18000 | 4 | 11920 |
| 1.15 | 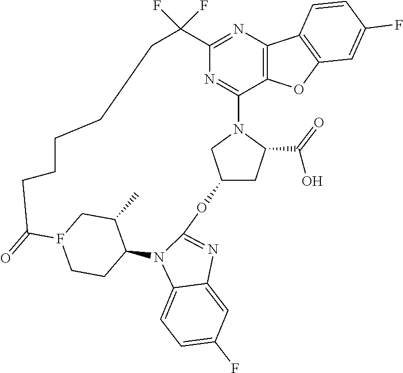 | 3.5 | 7.1 | >10000 | 3 | >1408 |
| 1.16 | 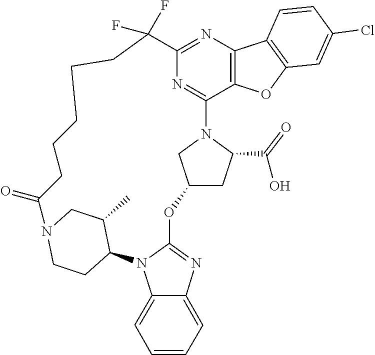 [SMILES: C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O] | 3.3 | 9.7 | 11000 | 10 | 1134 |
| 1.17 | 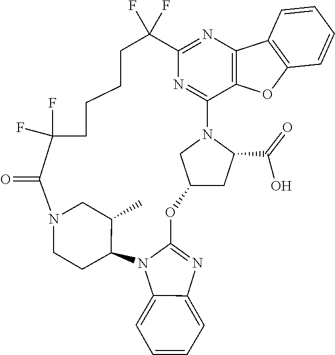 [SMILES: C[C@H]1CN2CC[C@@H]1n1c(nc3ccccc31)O[C@H]1C[C@@H](C(=O)O)N(C1)c1nc(nc3c1oc1ccccc13)C(F)(F)CCCCC(F)(F)C2=O] | 7.2 | 13 | 6600 | 8 | 519 |
| 1.18 | 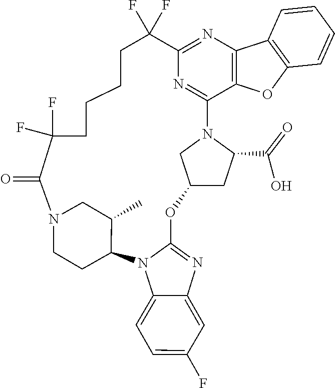 [SMILES: C[C@H]1CN2CC[C@@H]1n1c(nc3cc(F)ccc31)O[C@H]1C[C@@H](C(=O)O)N(C1)c1nc(nc3c1oc1ccccc13)C(F)(F)CCCCC(F)(F)C2=O] | 3.9 | 37 | 10000 | 10 | 270 |
| 1.19 | 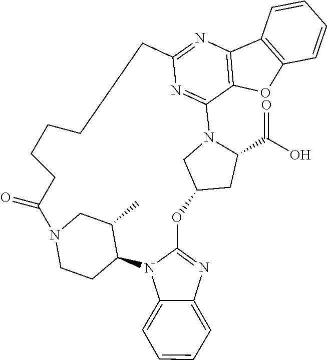 [SMILES: C[C@H]1CN2CC[C@@H]1n1c(nc3ccccc31)O[C@H]1C[C@@H](C(=O)O)N(C1)c1nc(nc3c1oc1ccccc13)CCCCCC2=O] | 3.8 | 40 | 11000 | 18 | 277 |
| 1.20 | 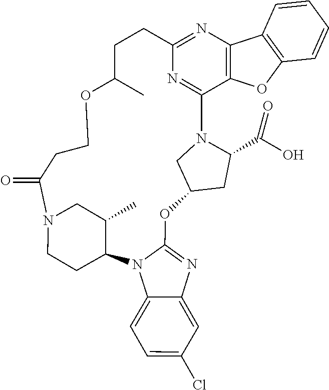 [SMILES: CC1CCc2nc(c3oc4ccccc4c3n2)N2C[C@H](C[C@H]2C(=O)O)Oc2nc3cc(Cl)ccc3n2[C@H]2CCN(C[C@@H]2C)C(=O)CCO1] | 4.9 | 13 | >10000 | 7 | >773 |
| 1.21 | 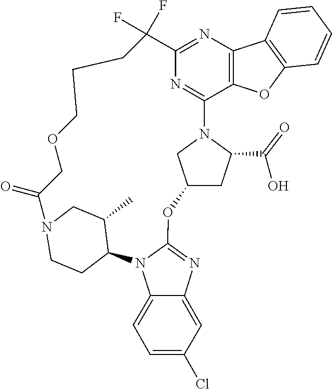 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)CCCOCC2=O] | 3.3 | 1.9 | >10000 | 1 | >5238 |
| 1.22 | 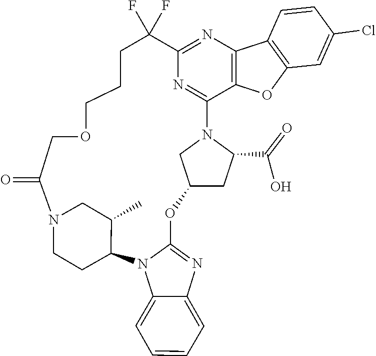 [SMILES: C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCOCC2=O] | 2.7 | 1.5 | >10000 | 4 | >6579 |
| 1.23 | 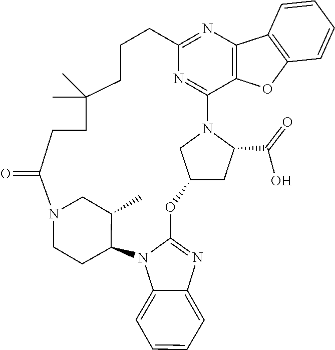 [SMILES: C[C@H]1CN2CC[C@@H]1n1c(nc3ccccc31)O[C@H]1C[C@@H](C(=O)O)N(C1)c1nc(nc3c1oc1ccccc13)CCCC(C)(C)CCC2=O] | 41 | 190 | 9500 | 143 | 51 |
| 1.24 | Example 1.24 is a mixture of the following two diastereoisomers: 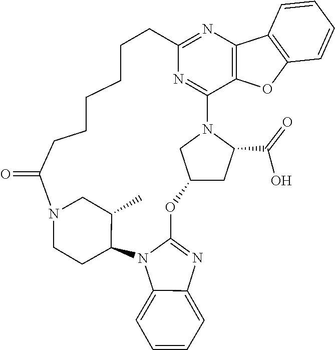 [SMILES: C[C@H]1CN2CC[C@@H]1n1c(nc3ccccc31)O[C@H]1C[C@@H](C(=O)O)N(C1)c1nc(nc3c1oc1ccccc13)CCCCCCC2=O] and 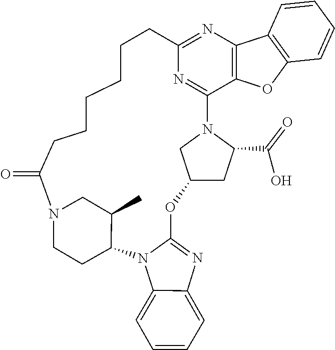 [SMILES: C[C@@H]1CN2CC[C@H]1n1c(nc3ccccc31)O[C@H]1C[C@@H](C(=O)O)N(C1)c1nc(nc3c1oc1ccccc13)CCCCCCC2=O] | 18 | 170 | 11000 | 86 | 65 |
| 1.25 | 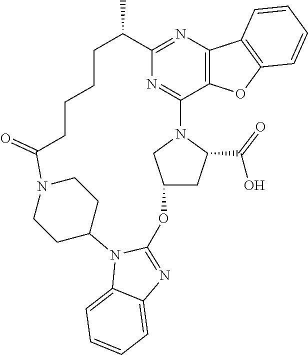 [SMILES: C[C@H]1CCCCC(=O)N2CCC(CC2)n2c(nc3ccccc32)O[C@H]2C[C@@H](C(=O)O)N(C2)c2nc1nc1c2oc2ccccc21] | 5.5 | 8 | >10000 | 41 | >1251 |
| 1.26 | 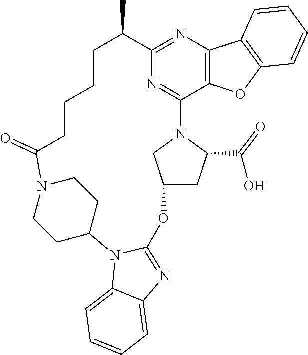 [SMILES: C[C@@H]1CCCCC(=O)N2CCC(CC2)n2c(nc3ccccc32)O[C@H]2C[C@@H](C(=O)O)N(C2)c2nc1nc1c2oc2ccccc21] | 3.4 | 5.5 | 15000 | 19 | 2687 |
| 1.27 | 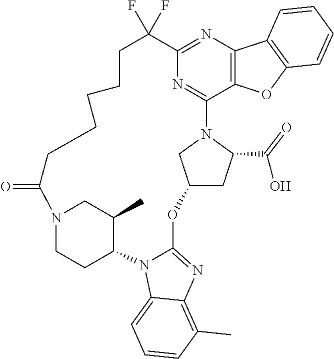 [SMILES: Cc1cccc2c1nc1n2[C@@H]2CCN(C[C@H]2C)C(=O)CCCCCC(F)(F)c2nc(c3oc4ccccc4c3n2)N2C[C@H](C[C@H]2C(=O)O)O1] | 100 | 180 | 6000 | 1584 | 33 |
| 1.28 | 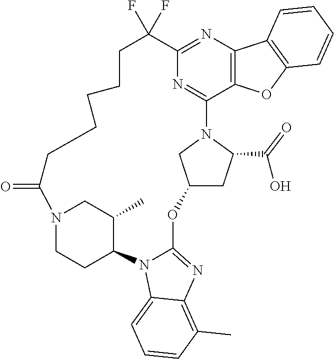 [SMILES: Cc1cccc2c1nc1n2[C@H]2CCN(C[C@@H]2C)C(=O)CCCCCC(F)(F)c2nc(c3oc4ccccc4c3n2)N2C[C@H](C[C@H]2C(=O)O)O1] | 11 | 15 | 4000 | 28 | 267 |
| 2.01 | Example 2.01 is a mixture of the following diastereoisomers: 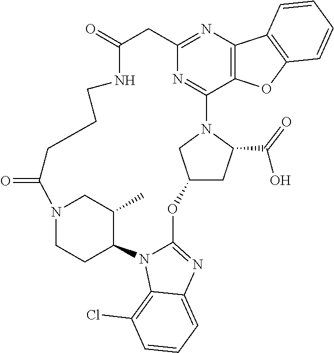 [SMILES: C[C@H]1CN2CC[C@@H]1n1c(nc3cccc(Cl)c31)O[C@H]1C[C@@H](C(=O)O)N(C1)c1nc(nc3c1oc1ccccc13)CC(=O)NCCCC2=O] and 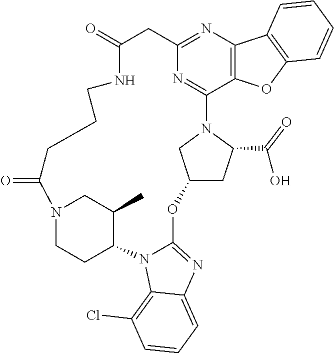 [SMILES: C[C@@H]1CN2CC[C@H]1n1c(nc3cccc(Cl)c31)O[C@H]1C[C@@H](C(=O)O)N(C1)c1nc(nc3c1oc1ccccc13)CC(=O)NCCCC2=O] | 6 | 11 | >10000 | 35 | >948 |
| 3.01 | 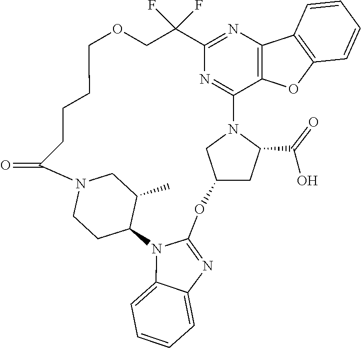 [SMILES: C[C@H]1CN2CC[C@@H]1n1c(nc3ccccc31)O[C@H]1C[C@@H](C(=O)O)N(C1)c1nc(nc3c1oc1ccccc13)C(F)(F)COCCCCC2=O] | 2.1 | 0.44 | 14000 | 1 | 32066 |
| 4.01 | 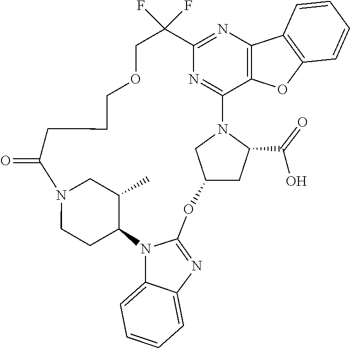 [SMILES: C[C@H]1CN2CC[C@@H]1n1c(nc3ccccc31)O[C@H]1C[C@@H](C(=O)O)N(C1)c1nc(nc3c1oc1ccccc13)C(F)(F)COCCCC2=O] | 1.7 | 0.69 | >10000 | 1 | >14442 |
| 5.01 | 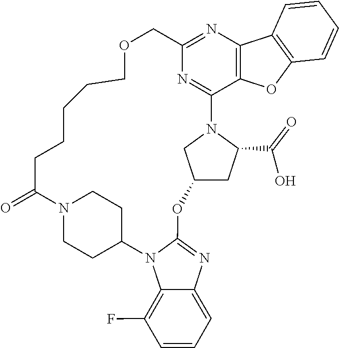 [SMILES: O=C(O)[C@@H]1C[C@H]2CN1c1nc(nc3c1oc1ccccc13)COCCCCCC(=O)N1CCC(CC1)n1c(nc3cccc(F)c31)O2] | 5.5 | 38 | >10000 | 33 | >260 |
| 6.01 | 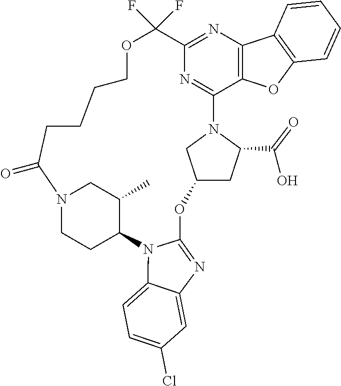 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O] | 3.5 | 3.1 | >10000 | 3 | >2225 |
| 7.01 | 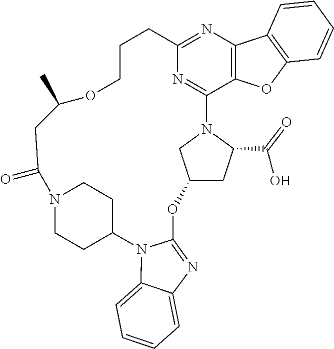 [SMILES: C[C@@H]1CC(=O)N2CCC(CC2)n2c(nc3ccccc32)O[C@H]2C[C@@H](C(=O)O)N(C2)c2nc(nc3c2oc2ccccc23)CCCO1] | 8.8 | 150 | 3500 | 101 | 23 |
| 8.01 | 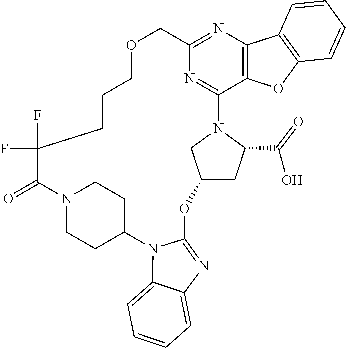 [SMILES: O=C(O)[C@@H]1C[C@H]2CN1c1nc(nc3c1oc1ccccc13)COCCCC(F)(F)C(=O)N1CCC(CC1)n1c(nc3ccccc31)O2] | 5.6 | 39 | >10000 | 35 | 255 |
| 9.01 | 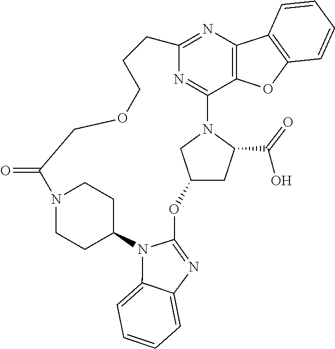 | 7.5 | 400 | n.d. | 41 | n.d. |
| 10.01 | 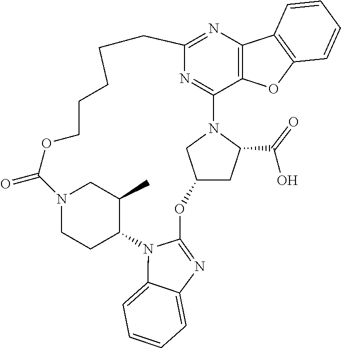 [SMILES: C[C@@H]1CN2CC[C@H]1n1c(nc3ccccc31)O[C@H]1C[C@@H](C(=O)O)N(C1)c1nc(nc3c1oc1ccccc13)CCCCCOC2=O] | 54 | 180 | 8200 | 439 | 45 |
| 10.02 | 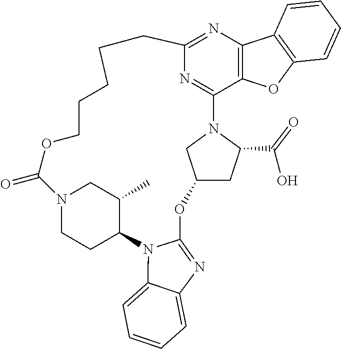 [SMILES: C[C@H]1CN2CC[C@@H]1n1c(nc3ccccc31)O[C@H]1C[C@@H](C(=O)O)N(C1)c1nc(nc3c1oc1ccccc13)CCCCCOC2=O] | 34 | 64 | >10000 | 83 | >156 |
| 11.01 | 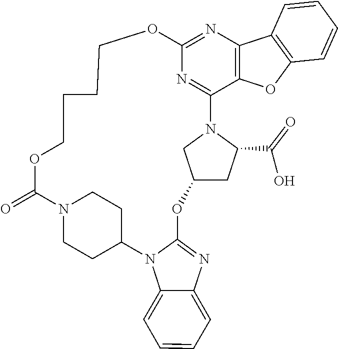 [SMILES: O=C(O)[C@@H]1C[C@H]2CN1c1nc(nc3c1oc1ccccc13)OCCCCOC(=O)N1CCC(CC1)n1c(nc3ccccc31)O2] | 10 | 180 | >1000 | 138 | >56 |
| 12.01 | Example 12.01 is a mixture of the following two diastereoisomers: 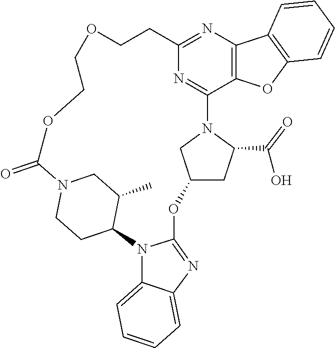 [SMILES: C[C@H]1CN2CC[C@@H]1n1c(nc3ccccc31)O[C@H]1C[C@@H](C(=O)O)N(C1)c1nc(nc3c1oc1ccccc13)CCOCCOC2=O] and 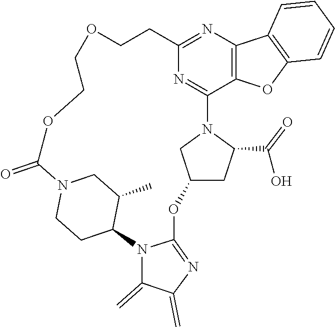 [SMILES: C=c1nc2n(c1=C)[C@H]1CCN(C[C@@H]1C)C(=O)OCCOCCc1nc(c3oc4ccccc4c3n1)N1C[C@H](C[C@H]1C(=O)O)O2] | 8.9 | 240 | >10000 | 225 | >42 |
| 13.01 | 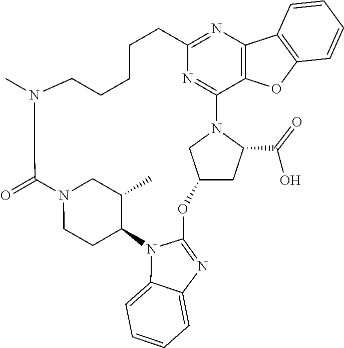 [SMILES: C[C@H]1CN2CC[C@@H]1n1c(nc3ccccc31)O[C@H]1C[C@@H](C(=O)O)N(C1)c1nc(nc3c1oc1ccccc13)CCCCCN(C)C2=O] | 34 | 500 | 7300 | 186 | 15 |

5.2 Comparison of the Example Compounds of Formula I, II or III with Prior Art Compounds

5.2.1 Compounds of WO 2020/142729

In WO 2020/142729 cGAS-inhibitors with partially similar structures have been disclosed.

On page 44 and 45 of WO 2020/142729 the “biochemical (in vitro) IC₅₀-values” with regard to cGAS-inhibition (corresponding to “hcGAS IC₅₀”) have been disclosed. Hereby compounds with a “biochemical (in vitro) IC₅₀-value” of less than 100 nM had been designated into “group A”, compounds with a “biochemical (in vitro) IC₅₀-value” of greater than 100 nM and less than 500 nM had been designated into “group B”, compounds with a “biochemical (in vitro) IC₅₀-value” of greater than 500 nM and less than 1 μM had been designated into “group C”, compounds with a “biochemical (in vitro) IC₅₀-value” of greater than 1 μM and less than 10 μM had been designated into “group D” and compounds with a “biochemical (in vitro) IC₅₀-value” of greater than 10 μM had been designated into “group E” (see page 44 of WO 2020/142729).

On page 45 of WO 2020/142729 it is disclosed that only compound No. 25 could be designated to “group A” having a “biochemical (in vitro) IC₅₀-value” of less than 100 nM. All other example compounds of WO 2020/142729 show “biochemical (in vitro) IC₅₀-values” of greater than 100 nM.

Selected prior art compounds of WO 2020/142729 including compound No. 25 have been synthesized and then have been tested with respect to their pharmacological properties regarding their potency to inhibit the cGAS/STING pathway using exactly the same assays as used for testing the compounds of the invention. In particular the “biochemical (in vitro) IC₅₀-values” with regard to cGAS-inhibition (hcGAS IC₅₀), the “cellular IC₅₀-values with regard to inhibition of IFN induction in virus-stimulated THP1 cells” (THP1₍ᵥᵢᵣ₎ IC₅₀), the “cellular IC₅₀-value with regard to inhibition of IFN induction in cGAMP-stimulated THP1 cells” (THP1_{(cGAMP)} IC₅₀) and the “IC₅₀-value with regard to inhibition of IFN induction in human whole blood” (hWB) have been experimentally determined for the structurally closest examples of WO 2020/142729 according to the assay methods as described in section 6 below (see Table 2).

TABLE 2
Pharmacological properties of a selection of Example compounds from WO 2020/142729

| Example No. | | | | | |
|---|---|---|---|---|---|
| (as disclosed | | hcGAS | | | hWB |
| in WO | | IC50 | THP1₍ᵥᵢᵣ₎ | THP1_{(cGAMP)} | IC50 |
| 2020/142729) | Structure | [nM] | IC50 [nM] | IC50 [nM] | [nM] |
| 15 | 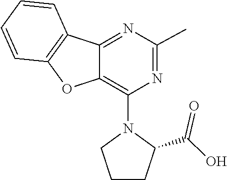 [SMILES: Cc1nc(N2CCC[C@H]2C(=O)O)c2oc3ccccc3c2n1] | 2700 | >17000 | >17000 | — |
| 25 | 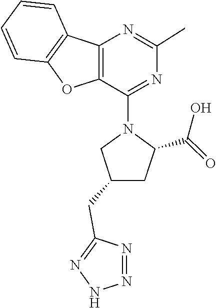 [SMILES: Cc1nc(N2C[C@@H](Cc3nn[nH]n3)C[C@H]2C(=O)O)c2oc3ccccc3c2n1] | 55 | >17000 | >17000 | >9992 |
| 28 | 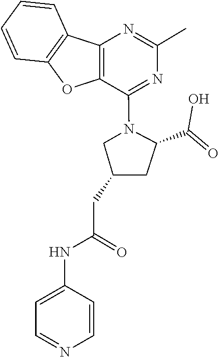 [SMILES: Cc1nc(N2C[C@@H](CC(=O)Nc3ccncc3)C[C@H]2C(=O)O)c2oc3ccccc3c2n1] | 630 | >32000 | >17000 | >9990 |
| 38 | 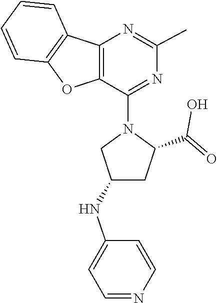 [SMILES: Cc1nc(N2C[C@@H](Nc3ccncc3)C[C@H]2C(=O)O)c2oc3ccccc3c2n1] | 3000 | >17000 | >17000 | >9990 |
| 58 | 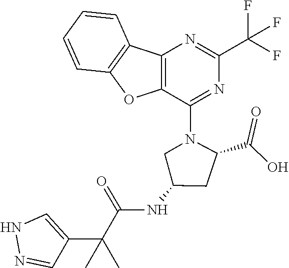 [SMILES: CC(C)(C(=O)N[C@H]1C[C@@H](C(=O)O)N(c2nc(C(F)(F)F)nc3c2oc2ccccc23)C1)c1cn[nH]c1] | 320 | 21000 | 23000 | >9982 |

The pharmacological properties for the Example compounds of the invention as summarized in Table 1 and the respective pharmacological properties for the compounds of WO 2020/142729 as summarized in Table 2 can be compared to each other, since they were experimentally determined according to the identical assay procedures as described in section 6 below.

From data as shown in Table 2 it is clear that all example compounds of WO 2020/142729 show “biochemical (in vitro) IC₅₀-values” (=hcGAS IC₅₀) that are significantly larger than 100 nM—with the only exception of Example No. 25 of WO 2020/142729 (in WO 2020/142729 designated in “Group A” having a “biochemical (in vitro) IC₅₀-value” (=hcGAS IC₅₀) of less than 100 nM). In contrast to that the Example compounds of the invention all have “biochemical (in vitro) IC₅₀-values” (hcGAS IC₅₀) of less than 100 nM. However, Example No. 25 of WO 2020/142729 which has a “biochemical (in vitro) IC₅₀-value” (hcGAS IC₅₀) of 55 nM, does not at all comply with the selection criterium of a “satisfying cellular inhibitory potency” shown by a THP1₍ᵥᵢᵣ₎ IC₅₀ of lower than 1 μM, because THP1₍ᵥᵢᵣ₎ IC₅₀ for Example No. 25 of WO 2020/142729 is 17 μM.

5.2.2 Compounds of WO 2022/174012

In WO 2022/174012 cGAS-inhibitors with partially similar structures have been disclosed.

On page 65 of WO 2022/174012 the “biochemical (in vitro) IC₅₀-values” with regard to cGAS-inhibition and on page 67 of WO 2022/174012 the “cellular IC₅₀-values” (IFNβ ELISA stimulated with THP-1) have been disclosed. Compound 5 (BBL0100455) of WO 2022/174012 seems to be the only compound of WO 2022/174012 that may have the potential to satisfy the selection criteria of the instant invention that means to have
a) an “biochemical (or enzymatic) (in vitro) IC₅₀-value” of smaller than 100 nM (in the “enzymatic assay of WO 2022/174012” compound 5 has been measured to fall into “group B” which represents an “enzymatic IC₅₀-value” of 50 nM to 100 nM”, see page 65, Table 2 of WO 2022/174012)
b) and to have a “cellular IC₅₀-value” (IFNβ ELISA stimulated with THP-1) of smaller than 1 μM (in the “cellular assay” of WO 2022/174012 compound 5 has been measured to fall into “group A” representing an “cellular IC₅₀-value” of <1 μM”, see page 67 and 68, Table 3 of WO 2022/174012).

However both, the biochemical/enzymatic assay and the cellular assays of WO 2022/174012, are not identical to the respective “biochemical/enzymatic assays and cellular assays” of the instant invention and therefore the measured biochemical/enzymatic IC₅₀-values and cellular IC₅₀-values of WO 2022/174012 are not comparable to the respective IC₅₀-values as measured for the compounds of the instant invention. Therefore compound 5 of WO 2022/174012 has been synthesized and then has been tested with respect to its pharmacological properties regarding its potency to inhibit the cGAS/STING pathway using exactly the same assays as used for testing the compounds of the instant invention and as described in Section 6 below.

TABLE 3
Pharmacological properties of Compound No. 5 of WO 2022/174012

| Example No. | | | | | |
|---|---|---|---|---|---|
| (as disclosed | | | | | hWB |
| in WO | | hcGAS | THP1₍ᵥᵢᵣ₎ | THP1_{(cGAMP)} | IC₅₀ |
| 2022/174012) | Structure | IC₅₀ [nM] | IC₅₀ [nM] | IC₅₀ [nM] | [nM] |
| Compound 5 (BBL0100455) | 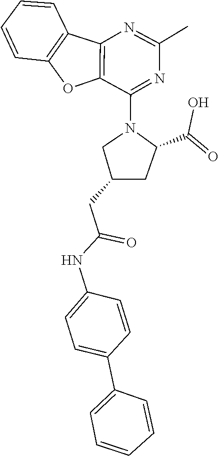 [SMILES: Cc1nc(N2C[C@@H](CC(=O)Nc3ccc(-c4ccccc4)cc3)C[C@H]2C(=O)O)c2oc3ccccc3c2n1] | 55 | > 10000 | > 10000 | |

As the data from Table 3 shows compound No. 5 (BBL0100455) of WO 2022/174012 has an acceptable biochemical/enzymatic IC₅₀-value of 55 nM (hcGAS IC₅₀=55 nM), but a cellular IC₅₀-value of larger than 10000 nM (THP1₍ᵥᵢᵣ₎ IC₅₀=10000 nM). Consequently the compound of the instant invention all are comparable to the compound No. 5 of WO 2022/174012 with respect to their biochemical/enzymatic IC₅₀-values, but are clearly superior over compound No. 5 of WO 2022/174012 with respect to their cellular IC₅₀-values (which are all smaller than 1000 nM for the compounds of formula I, II or III of the invention.

5.3 Prodrugs

It is known that esters of active agents with a carboxylic acid group may represent viable prodrugs which may e. g. show an improved oral absorption/bioavailability compared to the respective active agent. Frequently used prodrugs of active agents with a carboxylic acid group are for example methyl esters, ethyl esters, iso-propyl esters etc. (see Beaumont et al., Current Drug Metabolism, 2003, Vol. 4, Issue 6, 461-485).

Further, Nakamura et al., Bioorganic & Medicinal Chem., Vol. 15, Issue 24, p. 7720-7725 (2007), describes that also N-acylsulfonamide derivatives and N-acylsulfonylurea derivatives of a specific active agent with a free carboxylic acid group have the potential of being a viable prodrug.

Additionally, experimental hints have been found that also the methyl esters of the example compounds of formula I, II or III represent viable prodrugs of the cGAS inhibitors of formula I, II or III.

PCT/EP2022/062480 and PCT/EP2022/062496 (both so far unpublished) both disclose structurally similar cGAS-inhibitors as the cGAS-inhibitors of the instant invention which all comprise also carboxylic acid group attached to a pyrrolidine moiety. In both, PCT/EP2022/062480 and PCT/EP2022/062496 it has been experimentally shown that methyl esters derivatives of these cGAS-inhibitors carrying a carboxylic acid group attached to the pyrrolidine moiety act as viable prodrugs of the cGAS-inhibitors with the free carboxylic acid group.

Compounds P01, P02, P03 and P04 of PCT/EP2022/062480 were methyl ester derivatives and putative prodrugs of the respective Example compounds 4.04, 1.10, 1.12 and 3.14 of PCT/EP2022/062480 (which all had a free carboxylic group and were active cGAS-inhibitors with low biochemical IC₅₀-values and low cellular IC₅₀.values with regard to cGAS-inhibition).

Compounds P01, P02 and P03 of PCT/EP2022/062496 were methyl ester derivatives and putative prodrugs of the respective Example compounds 2.12, 1.13 and 1.05 of PCT/EP2022/062496 (which all had a free carboxylic group and were active cGAS-inhibitors with low biochemical IC₅₀-values and low cellular IC₅₀-values with regard to cGAS-inhibition).

In both, PCT/EP2022/062480 and PCT/EP2022/062496, the “active cGAS-inhibitors/Example compounds with their free carboxylic acid” and their “respective methyl ester derivatives/putative prodrugs” have been synthesized and have been tested for their pharmacological properties with respect to their potency to inhibit the cGAS/STING pathway.

This comparison of the properties of the Example compounds of PCT/EP2022/062480 and of PCT/EP2022/062496 with their free carboxylic acid on the one hand and the properties of their corresponding methyl ester derivatives/putative prodrugs shows that the “biochemical IC₅₀-values (hcGAS IC₅₀-values)” for the Example compounds are always around or even smaller than 10 nM, whereas the “biochemical IC₅₀-values” (hcGAS IC₅₀-values) for the corresponding methyl ester derivatives/prodrugs are always extremely large, that means generally larger than 7000 nM. That large difference between the IC₅₀-values of the Example compounds on the one hand and the IC₅₀-values of their corresponding methyl ester derivatives/prodrugs on the other hand has never been observed for the respective cellular IC₅₀-values (THP1₍ᵥᵢᵣ₎IC₅₀-values) which always stay more or less in the same range between example compounds and their corresponding prodrugs (see Table 4 below).

One possible explanation for that observation is that the Example compounds all have a free carboxylic group which seems to be crucial for inhibition of cGAS activity, whereas in all “methyl ester derivatives/prodrugs” the carboxyl group is masked by a carboxy-methyl ester group. Consequently, the methyl ester derivatives/prodrugs lose their inhibitory potency in the “in vitro human cGAS enzyme assay” (see section 6.1 below), because in this assay intracellular enzymes that cleave the carboxy-methyl ester group are absent and therefore the crucial free carboxylic acid group can not be restored in the biochemical assay. Therefore the prodrugs show extremely large “biochemical (in vitro) IC₅₀-values” (=hcGAS IC₅₀) in this “in vitro human cGAS enzyme assay”, whereas the corresponding Example compounds (which have a free carboxylic acid group from the beginning on) show small “biochemical (in vitro) IC₅₀-values” (=hcGAS IC₅₀).

In the cellular assay (=“human cGAS cell and the counter cell assay”, see section 6.2 below) endogenous cellular enzymes that cleave the carboxy-methyl ester group are present. Consequently not only the Example compounds of PCT/EP2022/062480 and of PCT/EP2022/062496 themselves (that already carry a free carboxylic acid group) show small THP1₍ᵥᵢᵣ₎IC₅₀-values, but also the corresponding methyl ester derivatives/prodrugs show relatively small “THP1₍ᵥᵢᵣ₎IC₅₀-values”, because in this “human cGAS cell assay” the carboxy-methyl ester group of the prodrugs can be cleaved by the endogenous intracellular enzymes and thereby will release the “active Example Compounds with the free carboxylic acid group” that shows cGAS-inhibitory potency again.

This explanation together with the measurements as shown in Table 4 imply that carboxy-methyl ester derivatives of the structurally similar Example compounds of PCT/EP2022/062480 and of PCT/EP2022/062496 really seem to represent viable prodrugs of the respective Example compound with the free carboxylic acid group (which themselves have no inhibitory potency regarding the in vitro human biochemical cGAS inhibition). However, upon cleavage of the carboxy-methyl ester by endogenous intracellular enzymes present in the cellular assays the “active Example Compounds” are restored, that exhibit again an inhibitory potency regarding the cGAS/STING pathway.

Since the Example Compounds of formula I, II or III of the present invention have the very same free carboxylic acid attached to the pyrrolidinyl moiety as the Example Compounds of PCT/EP2022/062480 or of PCT/EP2022/062496, it can be expected that carboxy-methyl ester derivatives of these Compounds of formula I, II or III will also act as prodrugs.

TABLE 4
Comparison between selected cGAS-inhibitor compounds as disclosed in
PCT/EP2022/062480 and PCT/EP2022/062496 and their respective methyl ester prodrugs:

| | | hcGAS | THP1₍ᵥᵢᵣ₎ | THP1_{(cGAMP)} | |
|---|---|---|---|---|---|
| Example No./ | | IC₅₀ | IC₅₀ | IC₅₀ | hWB |
| Prodrug No. | Structure | [nM] | [nM] | [nM] | IC₅₀ [nM] |
| P01 of PCT/EP2022/062 480) (Prodrug of Ex. 4.04 of PCT/EP 2022/062480) | 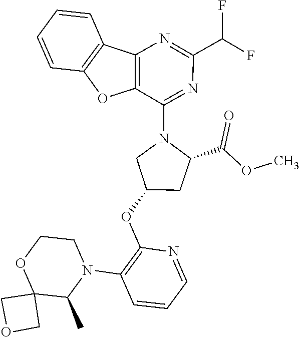 [SMILES: COC(=O)[C@@H]1C[C@H](Oc2ncccc2N2CCOC3(COC3)[C@@H]2C)CN1c1nc(C(F)F)nc2c1oc1ccccc12] | >9956 | 358 | 26058 | 1313 |
| Ex. 4.04 of PCT/EP2022/062 480) | 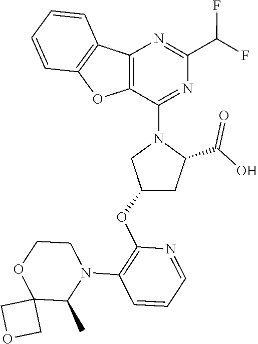 [SMILES: C[C@@H]1N(c2cccnc2O[C@H]2C[C@@H](C(=O)O)N(c3nc(C(F)F)nc4c3oc3ccccc34)C2)CCOC12COC2] | 2 | 386 | >16611 | 77 |
| P02 of PCT/EP2022/062 480) (Prodrug of Ex. 1.10 of PCT/ EP2022/062480) | 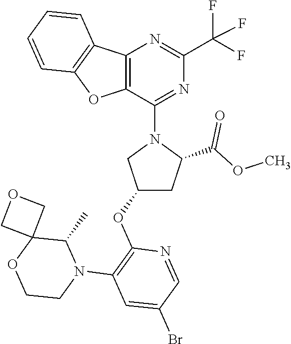 [SMILES: COC(=O)[C@@H]1C[C@H](Oc2ncc(Br)cc2N2CCOC3(COC3)[C@@H]2C)CN1c1nc(C(F)(F)F)nc2c1oc1ccccc12] | >9952 | 444 | 17109 | 5327 |
| Ex. 1.10 of PCT/EP2022/062 480) | 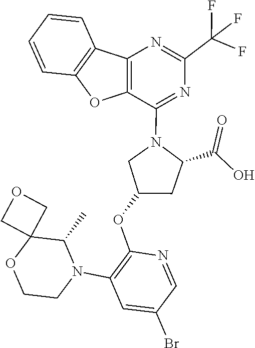 | 5 | 91 | 16130 | 33 |
| P03 of PCT/EP2022/062 480) (Prodrug of Ex. 1.12 of PCT/ EP2022/062480) | 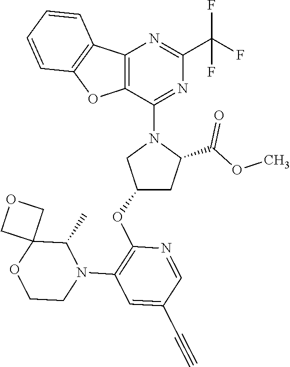 [SMILES: C#Cc1cnc(O[C@H]2C[C@@H](C(=O)OC)N(c3nc(C(F)(F)F)nc4c3oc3ccccc34)C2)c(N2CCOC3(COC3)[C@@H]2C)c1] | 9617 | 188 | 22845 | 1088 |
| Ex. 1.12 of PCT/EP2022/062 480) | 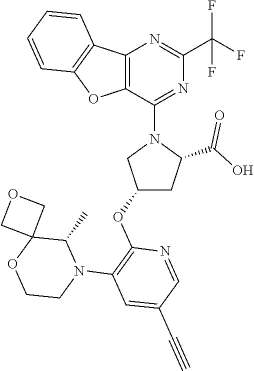 [SMILES: C#Cc1cnc(O[C@H]2C[C@@H](C(=O)O)N(c3nc(C(F)(F)F)nc4c3oc3ccccc34)C2)c(N2CCOC3(COC3)[C@@H]2C)c1] | 3 | 7 | 11892 | 15 |
| P04 of PCT/EP2022/062 480) (Prodrug of Ex. 3.14 of PCT/ EP2022/062480) | 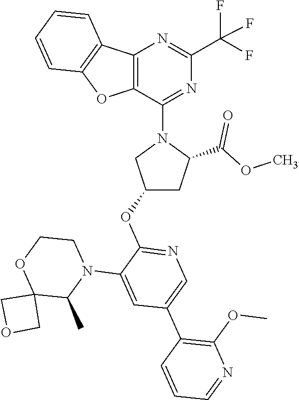 [SMILES: COC(=O)[C@@H]1C[C@H](Oc2ncc(-c3cccnc3OC)cc2N2CCOC3(COC3)[C@@H]2C)CN1c1nc(C(F)(F)F)nc2c1oc1ccccc12] | >9954 | 67 | >16621 | 2747 |
| Ex. 3.14 of PCT/EP2022/062 480) | 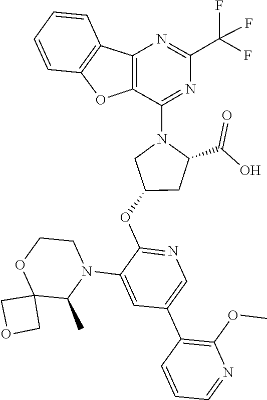 [SMILES: COc1ncccc1-c1cnc(O[C@H]2C[C@@H](C(=O)O)N(c3nc(C(F)(F)F)nc4c3oc3ccccc34)C2)c(N2CCOC3(COC3)[C@@H]2C)c1] | 11 | 10 | 7832 | 26 |
| P01 of PCT/EP2022/062 496) (Prodrug of Ex. 2.12 of PCT/ EP2022/062496) | 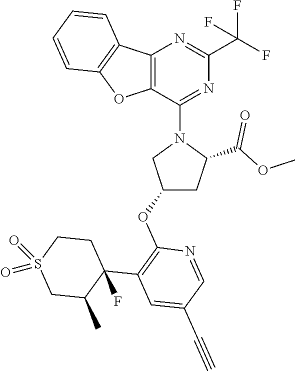 [SMILES: C#Cc1cnc(O[C@H]2C[C@@H](C(=O)OC)N(c3nc(C(F)(F)F)nc4c3oc3ccccc34)C2)c([C@@]2(F)CCS(=O)(=O)C[C@@H]2C)c1] | 19147 | 11 | >16612 | 67 |
| Ex. 2.12 of PCT/ EP2022/062496 | 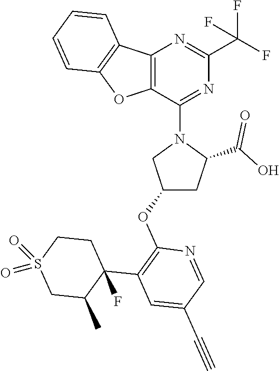 [SMILES: C#Cc1cnc(O[C@H]2C[C@@H](C(=O)O)N(c3nc(C(F)(F)F)nc4c3oc3ccccc34)C2)c([C@@]2(F)CCS(=O)(=O)C[C@@H]2C)c1] | 3 | 8 | 21781 | 8 |
| P02 of PCT/ EP2022/062496) (prodrug of Ex. 1.13 of PCT/ EP2022/062496) | 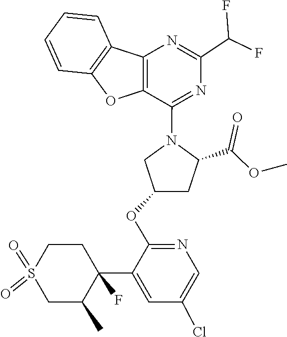 [SMILES: COC(=O)[C@@H]1C[C@H](Oc2ncc(Cl)cc2[C@@]2(F)CCS(=O)(=O)C[C@@H]2C)CN1c1nc(C(F)F)nc2c1oc1ccccc12] | >9954 | 29 | >16620 | 140 |
| Ex. 1.13 of PCT/ EP2022/062496 | 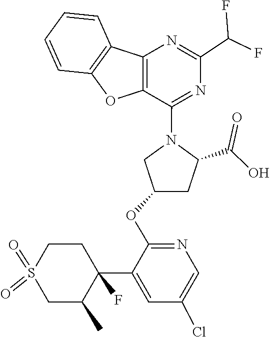 [SMILES: C[C@H]1CS(=O)(=O)CC[C@]1(F)c1cc(Cl)cnc1O[C@H]1C[C@@H](C(=O)O)N(c2nc(C(F)F)nc3c2oc2ccccc23)C1] | 2 | 6 | 22502 | 6 |
| P03 of PCT/ EP2022/062496) (prodrug of Ex. 1.05 of PCT/ EP2022/062496) | 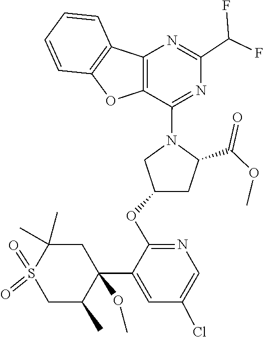 [SMILES: COC(=O)[C@@H]1C[C@H](Oc2ncc(Cl)cc2[C@@]2(OC)CC(C)(C)S(=O)(=O)C[C@@H]2C)CN1c1nc(C(F)F)nc2c1oc1ccccc12] | 7253 | 202 | 20592 | 1994 |
| Ex. 1.05 of PCT/ EP2022/062496 | 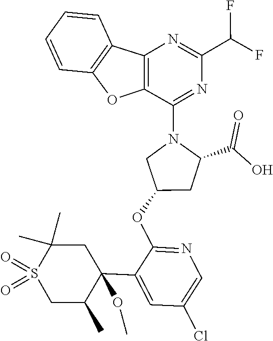 [SMILES: CO[C@]1(c2cc(Cl)cnc2O[C@H]2C[C@@H](C(=O)O)N(c3nc(C(F)F)nc4c3oc3ccccc34)C2)CC(C)(C)S(=O)(=O)C[C@@H]1C] | 2 | 44 | 13188 | 135 |

### 5. BIOLOGICAL EXPERIMENTS

The activity of the compounds of the invention may be demonstrated using the following in vitro cGAS enzyme and cell assays:

6.1 Method: Human cGAS Enzyme Assay (hcGAS IC₅₀ (In Vitro))

Human cGAS enzyme was incubated in the presence of a 45 base pair double stranded DNA to activate the enzyme and GTP and ATP as substrates. Compound activity was determined by measuring the effect of compounds on the formation of the product of the enzyme reaction, cGAMP, which is measured by a mass spectrometry method.

Enzyme Preparation:

HumancGAS (amino acid 1-522) with an N-terminal 6×-His-tag and SUMO-tag was expressed in E. coli BL21(DE3) pLysS (Novagen) cells for 16 h at 18° C. Cells were lysed in buffer containing 25 mM Tris (pH 8), 300 mM NaCl, 10 mM imidazole, 10% glycerol, protease inhibitor cocktail (cOmplete™, EDTA-free, Roche) and DNase (5 μg/mL). The cGAS protein was isolated by affinity chromatography on Ni-NTA agarose resin and further purified by size exclusion chromatography using a Superdex 200 column (GE Healthcare) equilibrated in 20 mM Tris (pH 7.5), 500 mM KCl, and 1 mM TCEP. Purified protein was concentrated to 1.7 mg/mL and stored at −80° C.

Assay Method

Compounds were delivered in 10 mM DMSO solution, serially diluted and transferred to the 384 well assay plate (Greiner #781201) using an Echo acoustic dispenser. Typically, 8 concentrations were used with the highest concentration at 10 μM in the final assay volume followed by ^{˜}1:5 dilution steps. DMSO concentration was set to 1% in the final assay volume. The 384 well assay plate contained 22 test compounds (column 1-22), and DMSO in column 23 and 24.

After the compound transfer, 15 μL of the enzyme-DNA-working solution (12 nM cGAS, 0.32 μM 45base pair DNA in assay buffer, 10 mM Tris pH 7.5/10 mM KCl/5 mM MgCl₂/1 mM DTT) were added to each well from column 1-23 via a MultiDrop Combi dispenser. In column 24, 15 μl of assay buffer without enzyme/DNA were added as a low control.

The plates were then pre-incubated for 60 min at room temperature.

Following that, 10 μL of GTP (ThermoFisher #R0461)-ATP (Promega #V915B) mix in assay buffer were added to the assay plate (columns 1-24, 30 μM final concentration each) using a Multidrop Combi.

The plates were incubated again for 90 min at room temperature.

Following the incubation, the reaction was stopped by 80 μL of 0,1% formic acid in assay buffer containing 5 nM cyclic-di-GMP (Sigma #SML1228) used as internal standard for the mass spectrometry. The total volume/well was 105 μL.

Rapidfire MS Detection

The plates were centrifuged at 4000 rpm, 4° C., for 5 min.

The RapidFire autosampler was coupled to a binary pump (Agilent 1290) and a Triple Quad 6500 (ABSciex, Toronto, Canada). This system was equipped with a 10 μL loop, C18 [12 IL bed volume] cartridge (Agilent, Part No. G9210A) containing 10 mM NH4Ac (aq) water (pH7.4) as eluent A (pump 1 at 1.5 mL/min, pump 2 at 1.25 mL/min) and 10 mM NH4Ac in v/v/v 47.5/47.5/5 ACN/MeOH/H₂O (pH7.4) as eluent B (pump 3 at 1.25 mL/min). Aspiration time: 250 ms; Load time: 3000 ms; Elute time: 3000 ms; Wash volume: 500 μL.

The MS was operated in positive ion mode with HESI ion source, with a source temperature of 550 C, curtain gas=35, gas 1=65, and gas 2=80. Unit mass resolution in SRM mode. The following transitions and MS parameters (DP: declustering potential and CE: collision energy) for cGAMP and DicGMP were determined:

Analyte: cGAMP at 675.1/524, DP=130, CE=30 and

Internal standard: cyclic-di-GMP at 690.1/540, DP=130, CE=30.

The formation of cGAMP was monitored and evaluated as ratio to cyclic-di-GMP.

Data Evaluation and Calculation:

For data evaluation and calculation, the measurement of the low control was set as 0% control and the measurement of the high control was set as 100% control. The IC₅₀ values were calculated using the standard 4 parameter logistic regression formula. Calculation: [y=(a−d)/(1+(x/c)\hat{ }b)+d], a=low value, d=high value; x=conc M; c=1C50 M; b=slope

6.2 Method: Human cGAS Cell Assay and cGAMP Stimulated Counter Cell Assay (THP1₍ᵥᵢᵣ₎ IC₅₀ and THP1_{(cGAMP)} IC₅₀)

THP1-Dual™ cells (InvivoGen #thpd-nfis) expressing IRF dependent Lucia luciferase reporter were used as basis for both assays. For the detection of cellular cGAS activity cells were stimulated by a baculovirus (pFastbac-1, Invitrogen, no coding insert) infection that delivers the cGAS enzyme stimulating double-stranded DNA (measurement of THP1₍ᵥᵢᵣ₎ IC₅₀).

For the counter assay, cells were stimulated by cGAMP (SigmaAldrich #SML1232) to activate the identical pathway independent and directly downstream of cGAS (measurement of THP1_{(cGAMP)} IC₅₀).

Pathway activity was monitored by measuring the Lucia luciferase activity induced by either DNA stimulated cGAS enzyme activity (measurement of THP1₍ᵥᵢᵣ₎ IC₅₀) or by cGAMP directly (measurement of THP1_{(cGAMP)} IC₅₀, counter assay).

Assay Method

Compounds were delivered in 10 mM DMSO solution, serially diluted and transferred to the 384 well assay plate (Greiner #781201) using an Echo acoustic dispenser. Typically, 8 concentrations were used with the highest concentration at 10 μM in the final assay volume followed by ^{˜}1:5 dilution steps. DMSO concentration was set to 1% in the final assay volume. The 384 well assay plate contained 21 test compounds (column 1-22), and DMSO in column 23 and 24.

Cells, cultivated according to manufacturer conditions, were harvested by centrifugation at 300 g/10 min and were then resuspended and diluted to 1.66E5 cells/ml in fresh cell culture medium (RPMI 1640 (Gibco #A10491-01), 10% FCS (Gibco #10500), 1× GlutaMax (Gibco #35050-061), 1× Pen/Strep solution (Gibco #15140-122), 100 μg/ml Normocin (InvivoGen #ant-nr), 100 μg/ml Zeocin (InvivoGen #ant-zn), 10 μg/ml Blasticidin S (Life Technologies #A11139-03)). The baculovirus solution was then added 1:200 (have varied according to virus batch) to the cells (measurement of THP1₍ᵥᵢᵣ₎ IC₅₀). Alternatively, for the counter assay cGAMP was added to the cells at a final concentration of 10 μM (measurement of THP1_{(cGAMP)} IC₅₀).

30 μL of the cell/virus-mix were added to each well of the compound plate from column 1-23 via MultiDrop Combi dispenser (5000 cells/well). In column 24, 30 μl/5000 cells/well without virus were added as a low control.

The plates were then incubated for 18 h at 37° C. in a humidified incubator.

Following that, 15 μL of QuantiLuc detection reagent (InvivoGen #rep-qlcg5) were added to each well using a MultiDrop Combi. Measurement was done immediately after the addition using an EnVision reader (US-luminescence read-mode).

Data Evaluation and Calculation:

For data evaluation and calculation, the measurement of the low control was set as 0% control and the measurement of the high control was set as 100% control. The IC₅₀ values were calculated using the standard 4 parameter logistic regression formula. Calculation: [y=(a−d)/(1+(x/c)\hat{ }b)+d], a=low value, d=high value; x=conc M; c=IC50 M; b=slope

6.3 Method: Human Whole Blood Assay (Human WB IC₅₀)

For the detection of cellular cGAS activity human whole blood was stimulated by transfection with double stranded DNA. Pathway activity was monitored by measuring the IFNα2α production.

Assay Method

Compounds were delivered as 10 mM DMSO solution and serially diluted and transferred to the 96-well cell culture plate (Corning #3595), prefilled with 20 μl OptiMEM (Gibco, #11058-021) in each well, using an Echo acoustic dispenser. Typically, 8 concentrations were used with the highest concentration at 10 μM in the final assay volume followed by ^{˜}1:5 dilution steps. DMSO concentration was set to 0.1% in the final assay volume. The 96-well assay plate contained 10 test compounds, and DMSO in control wells.

Collection of human whole blood from 3 or more healthy donors (male or female, no medication for 7 days except contraceptive and thyroxine) as Na-Citrate blood (e.g. 3.8% in Monovettes from Sarstedt) was conducted in parallel. Whole blood was kept at room temperature for a maximum of 3 hours after collection until use in the assay. 160 μl of the whole blood samples was transferred to each well of the 96-well assay plates filled with compound/OptiMEM. All assay plates were prepared as duplicates with blood from different donors. Blood plates were kept at room temperature for 60 minutes and continuous shaking with 450 rpm, covered with the lid, but not sealed.

DNA-Fugene mix (Herring DNA, Sigma Aldrich #D6898-1G, Fugene (5×1 mL), Promega #E2312) was prepared in OptiMEM and incubated for 10 min at RT (125 ng DNA/20 μl and Fugene ratio 9.6:1). 20 μl of the DNA Fugene mix was added to each well, resulting in 125 ng DNA/well/200 μl, and Fugene Ratio 9.6:1. 20 μl OptiMEM and 9.6:1 Fugene was added to all low control wells.

After covering assay plates with aera seals and the lid, blood plates were kept at room temperature for 30 minutes and continuous shaking with 450 rpm, followed by an overnight incubation of 22 h at 37° C. in the incubator, without shaking.

For the detection of IFNα-2α in human plasma, the biotinylated capture antibody (Antibody set IFNA2, Meso Scale Diagnostics #B21VH-3, including coating and capture antibody) was diluted 1:17.5 in Diluent 100 (Meso Scale Diagnostics #R50AA-4), according to the manufacturer's directions. U-Plex MSD GOLD 96-well Small Spot Strepavidin SECTOR Plates (Meso Scale Diagnostics #L45SA-5) were coated with 25 μl diluted capture antibody. Coated plates were incubated for 60 min at room temperature under continuous shaking at 700 rpm. MSD IFNα-2α plates were washed three times with 150 μl wash buffer (1× HBSS, 0.05% Tween).

After blocking the plates with 100 μl block solution/well (1× HBSS with 0.2% Tween, 2% BSA) for 60 min at room temperature and continuous shaking at 700 rpm, plates were emptied as dry as possible by dumping just before continuing with the human plasma.

Whole Blood assay plates were centrifuged at 1600 rpm for 10 minutes. 25 μl of supernatant was transferred with a pipetting robot from each whole blood plate to the corresponding IFNα-2α plate. Plates were sealed with microplate seals and kept at room temperature again under continuous shaking at 700 rpm for two hours.

Next MSD IFNα-2α plates were washed three times with 150 μl wash buffer (1× HBSS, 0.05% Tween), before adding 25 μl MSD SULFO-TAG IFNα-2α Antibody solution (1:100 diluted in Diluent 3 (Meso Scale Diagnostics #R50AP-2) to each well of the plates.

Afterwards plates were sealed with microplate seals and kept at room temperature again under continuous shaking at 700 rpm for two hours. Finally, MSD IFNα-2α plates were washed three times with 150 μl wash buffer (1× HBSS, 0.05% Tween). 150 μl 2× Read buffer was added to each well and plates were immediately measured with the MSD Sector S600 Reader using the vendor barcode.

Data Evaluation and Calculation:

For data evaluation and calculation, % control calculation of each well was based on the mean of high (DNA stimulated control) and mean of low (unstimulated control) controls by using the following formula:

[counts(sample)−counts(low))/(counts(high)−counts(low))]*100

The IC₅₀-values were calculated using the standard 4 parameter logistic regression formula.

Calculation: [y=(a−d)/(1+(x/c)\hat{ }b)+d], a=low value, d=high value; x=conc M; c=IC₅₀ M; b=slope

### 6. INDICATIONS

As has been found, the compounds of formula I, II or III are characterized by their range of applications in the therapeutic field. Particular mention should be made of those applications for which the compounds of formula I, II or III according to the invention are preferably used on the basis of their pharmaceutical activity as cGAS inhibitors. While the cGAS pathway is important for host defense against invading pathogens, such as viral infection and invasion by some intracellular bacteria, cellular stress and genetic factors may also cause production of aberrant cellular dsDNA, e.g. by nuclear or mitochondrial leakage, and thereby trigger autoinflammatory responses. Consequently, cGAS inhibitors have a strong therapeutic potential to be used in the treatment of diverse autoinflammatory and autoimmune diseases.

An et al., Arthritis Rheumatol. 2017 April; 69(4):800-807, disclosed that cGAS expression in peripheral blood mononuclear cells (PBMCs) was significantly higher in patients with the autoimmune disease systemic lupus erythematosus (SLE) than in normal controls. Targeted measurement of cGAMP by tandem mass spectrometry detected cGAMP in 15% of the tested SLE patients, but none of the normal or rheumatoid arthritis controls. Disease activity was higher in SLE patients with cGAMP versus those without cGAMP. Whereas higher cGAS expression may be a consequence of exposure to type I interferon (IFN), detection of cGAMP in SLE patients with increased disease activity indicates potential involvement of the cGAS pathway in disease expression.

Park et al., Ann Rheum Dis. 2018 October; 77(10):1507-1515, also discloses the involvement of the cGAS pathway in the development of SLE.

Thim-Uam et al., iScience 2020 Sep. 4; 23(9), 101530 (doi: 10.1016/j.isci.2020.101530), discloses that the STING pathway mediates lupus via the activation of conventional dendritic cell maturation and plasmacytoid dendritic cell differentiation.

Gao et al., Proc. Natl. Acad. Sci. USA. 2015 Oct. 20; 112(42):E5699-705, describes that the activation of cGAS by self-DNA leads to certain autoimmune diseases such as interferonopathies.

Tonduti et al., Expert Rev. Clin. Immunol. 2020 February; 16(2):189-198 discloses that cGAS inhibitors have particular therapeutic potential in Aicardi-Goutières syndrome and familial chilblain lupus, which are lupus-like severe autoinflammatory immune-mediated disorders.

Steiner et al., Nat Commun. 2022 Apr. 28; 13(1):232; doi: 10.1038, shows that deficiency in coatomer complex I causes aberrant activation of STING signalling and COPA syndrome, and that cGAS is required to drive type I IFN signalling in a COPA syndrome cell model.

Li et al show that plasma-derived DNA containing-extracellular vesicles induce STING-mediated proinflammatory responses in dermatomyositis (Theranostics. 2021; 11(15): 7144-7158). Zhou et al (J Clin Lab Anal. 2022 October; 36(10): e24631) describes a correlation between activation of cGAS-STING pathway and myofiber atrophy/necrosis in dermatomyositis.

In Yu et al., Cell 2020 Oct. 29; 183(3):636-649, the link between TDP-43 triggered mitochondrial DNA and the activation of the cGAS/STING pathway in amyotrophic lateral sclerosis (ALS) is described.

Ryu et al., Arthritis Rheumatol. 2020 November; 72(11):1905-1915, also shows that bioactive plasma mitochondrial DNA is associated with disease progression in specific fibrosing diseases such as systemic sclerosis (SSc) or interstitial lung deseases (ILDs), progressive fibrosing interstitial lung diseases (PF-ILDs), and idiopathic pulmonary fibrosis (IPF).

In Schuliga et al., Clin. Sci. (Lond). 2020 Apr. 17; 134(7):889-905, it is described that self-DNA perpetuates IPF lung fibroblast senescence in a cGAS-dependent manner.

Additional scientific hints linking the cause for other fibrosing diseases such as non-alcoholic steatohepatitis (NASH) with the cGAS/STING pathway have been described in Yu et al., J. Clin. Invest. 2019 Feb. 1; 129(2):546-555, and in Cho et al., Hepatology. 2018 October; 68(4): 1331-1346.

Nascimento et al., Sci. Rep. 2019 Oct. 16; 9(1):14848, discloses that self-DNA release and STING-dependent sensing drives inflammation due to cigarette smoke in mice hinting at a link between the cGAS-STING pathway and chronic obstructive pulmonary disease (COPD).

Ma et al., Sci. Adv. 2020 May 20; 6(21):eaaz6717, discloses that ulcerative colitis and inflammatory bowel disease (IBD) may be restrained by controlling cGAS-mediated inflammation.

Gratia et al., J. Exp. Med. 2019 May 6; 216(5):1199-1213, shows that Bloom syndrome protein restrains innate immune sensing of micronuclei by cGAS. Consequently cGAS-inhibitors have a therapeutic potential in treating Bloom's syndrome.

Kerur et al., Nat. Med. 2018 January; 24(1):50-61, describes that cGAS plays a significant role in noncanonical-inflammasome activation in age-related macular degeneration (AMD).

Visitchanakun et al., Int J Mol Sci. 2021 Oct. 23; 22(21):11450, shows that GAS deficient mice were less severe than the wildtype mice in the cecal ligation and puncture (CLP) and lipopolysaccharide (LPS) injection sepsis models.

Wang et al., Mediators Inflamm. 2015; 2015:192329, describes that cGAS Is required for cell proliferation and Inflammatory cytokine production in rheumatoid arthritis synoviocytes. It has also bee reported that in an inflammatory arthritis mouse model, cGAS deficiency suppressed interferon responses, inflammatory cell infiltration and joint swelling (Willemsen et al., Cell Rep. 2021 Nov. 9; 37(6):109977).

Guo et al., Osteoarthritis Cartilage. 2021 August; 29(8):1213-1224, described that damaged DNA is a key pathologic factor for osteoarthritis (OA) and this is likely mediated by the cGAS/STING pathway, since STING knockdown alleviated destabilization of the medial meniscus-induced OA development in mice.

Mao et al., Arterioscler Thromb Vasc Biol (2017) 37(5):920-929, shows that the cGAS/STING pathway mediates endothelial inflammation in response to free fatty acid-Induced Mitochondrial damage in diet-Induced obesity, indicating that cGAS inhibitors have also the potential in the treatment of obesity and diabetes.

Kerur et al, Nat Med. 2018 January; 24(1):50-61, describes that cGAS levels were elevated in the retinal pigmented epithelium in human eyes with geographic atrophy, and cGAS drives activation of noncanonical-inflammasome activation in age-related macular degeneration.

cGAS promotes cellular senescence and senescence-associated secretory phenotype (Yang et al, Proc Natl Acad Sci USA 2017 Jun. 6; 114:E4612-E4620). Cytoplasmic chromatin triggers inflammation in senescence through cGAS/STING, and STING null-mice have reduced tissue inflammation abd aging (Dou et al, Nature. 2017 550: 402-406). Furthermore, in humans a variation within the STING gene is associated with healthy aging, most likely due to a decreased inflammaging (Hamann et al, Gerontology 2019; 65:145-154). Taken together, a STING inhibitor will reduce senescence associated inflammation and senescent cell accumulation and will leads improvement in senescence associated diseases such as aging, muscle disorders and fibrosis.

Further, the cGAS inhibitors of formula I, II or III also have a therapeutic potential in the treatment of cancer (see Hoong et al., Oncotarget. 2020 Jul. 28; 11(30):2930-2955, and Chen et al., Sci. Adv. 2020 Oct. 14; 6(42):eabb8941).

Additionally, the cGAS inhibitors of formula I, II or III have also a therapeutic potential in the treatment of heart failure (Hu et al., Am. J. Physiol. Heart Circ. Physiol. 2020 Jun. 1; 318(6):H1525-H1537).

Further scientific hints at a correlation between Parkinsons disease and the cGAS/STING pathway (Sliter et al., Nature. 2018 September; 561(7722):258-262) and between Sjogren's syndrome and the cGAS/STING pathway (Papinska et al., J. Dent. Res. 2018 July; 97(8):893-900) exist.

Furthermore, cGAS inhibitors of formula I, II or III have also a therapeutic potential in the treatment of COVID-19/SARS-CoV-2 infections as shown in Di Domizio et al., Nature. 2022 Jan. 19. doi: 10.1038/s41586-022-04421-w: “The cGAS-STING pathway drives type I IFN immunopathology in COVID-19”, and in Neufeldt et al., Commun Biol. 2022 Jan. 12; 5(1):45. doi: 10.1038/s42003-021-02983-5: “SARS-CoV-2 infection induces a pro-inflammatory cytokine response through cGAS-STING and NF-kappaB”.

Additionally, cGAS inhibitors of formula I, II or III have a therapeutic potential in the treatment of renal inflammation and renal fibrosis as shown in Chung et al., Cell Metab. 2019 30:784-799: “Mitochondrial Damage and Activation of the STING Pathway Lead to Renal Inflammation and Fibrosis”, and in Maekawa et al., Cell Rep. 2019 29:1261-1273: “Mitochondrial Damage Causes Inflammation via cGAS-STING Signaling in Acute Kidney Injury”.

Furthermore, cGAS inhibitors of formula I, II or III have a therapeutic potential in the treatment of cancer as shown in Bakhoum et el., Nature. 2018 Jan. 25; 553(7689):467-472: “Chromosomal instability drives metastasis through a cytosolic DNA response”, and in Liu et al., Nature. 2018 November; 563(7729):131-136: “Nuclear cGAS suppresses DNA repair and promotes tumorigenesis”.

Additionally, cGAS inhibitors of formula I, II or III have a therapeutic potential in the treatment of dysmetabolism, because STING^{gt} animals show reduced macrophage infiltration in adipose tissue upon subchronic high caloric intake (HFD) and STING^{gt} and IRF3-deficiency leads to a decrease in blood glucose and insulin and reduced body weight (Mao et al, Arterioscler Thromb Vasc Biol, 2017; 37 (5): 920-929).

Furthermore, cGAS inhibitors of formula I, II or III have a therapeutic potential in the treatment of vascular diseases and leads to vascular repair/regeneration, because the release of mitochondrial DNA into the cytosol of endothelial cells results in cGAS/STING pathway activation and suppression of endothelial proliferation. Further, knockout of the cGAS gene restores endothelial repair/regeneration in a mouse model of inflammatory lung injury (Huang et al, Immunity, 2020, March 2017; 52 (3): 475-486.e5. doi: 10.1016/j.immuni.2020,02.002).

Additionally, cGAS inhibitors of formula I, II or III have a therapeutic potential in the treatment of age-related and obesity-related cardiovascular diseases (Hamann et al, Immun Ageing, 2020 Mar. 14; 17: 7; doi: 10.1186/s12979-020-00176-y.eCollection 2020).

Consequently the compounds of formula I, II or III as cGAS inhibitors can be used in the therapy of autoinflammatory and autoimmune diseases such as systemic lupus erythematosus (SLE), interferonopathies, Aicardi-Goutières syndrome(AGS), COPA syndrome, familial chilblain lupus, age-related macular degeneration (AMD), amyotrophic lateral sclerosis (ALS), inflammatory bowel disease (IBD), chronic obstructive pulmonary disease (COPD), Bloom's syndrome, Sjogren's syndrome, rheumatoid arthritis and Parkinson disease.

Additionally the compounds of formula I, II or III as cGAS inhibitors can be used in the therapy of fibrosing disease such as systemic sclerosis (SSc), interferonopathies, non-alcoholic steatotic hepatitis (NASH), interstitial lung disease (ILD), preferably progressive fibrosing interstitial lung disease (PF-ILD), in particular idiopathic pulmonary fibrosis (IPF).

Further, the compounds of formula I, II or III as cGAS inhibitors can be used in the therapy of age-related macular degeneration (AMD), retinopathy, glaucoma, diabetes, obesity, aging, muscle disorders, sepsis, osteoarthritis, heart failure, COVID-19/SARS-CoV-2 infection, renal inflammation, renal fibrosis, dysmetabolism, vascular diseases, cardiovascular diseases and cancer.

### 7. COMBINATIONS

The compounds of formula I, II or III may be administered to the patient alone or in combination with one or more other pharmacologically active agents.

In a preferred embodiment of the invention the compounds of formula I, II or III may be combined with one or more pharmacologically active agents selected from the group of anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitiors, anti-interleukin antibodies, non-specific immunotherapeutics such as interferones or other cytokines/chemokines, cytokine/chemokine receptor modulators (i.e. cytokine receptor agonists or antagonists), Toll-like receptor agonists (=TLR agonists), immune checkpoint regulators, anti-TNF antibodies (Humira™), and anti-BAFF agents (Belimumab and Etanercept).

Anti-fibrotic agents are preferably selected from Pirfenidone and tyrosine kinase inhibitors such as Nintedanib, wherein Nintedanib is preferred in particular.

Preferred examples of anti-inflammatory agents are NSAIDs and corticosteroids.

NSAIDs are preferably selected from ibuprofen, naproxen, diclofenac, meloxicam, celecoxib, acetylsalicylic acid (Aspirin™), indomethacin, mefenamic acid and etoricoxib.

Corticosteroids are preferably selected from Flunisolide, Beclomethasone, Triamcinolone, Budesonide, Fluticasone, Mometasone, Ciclesonide, Rofleponide and Dexametasone.

Antiallergic agents/anti-histamines are preferably selected from Epinastine, Cetirizine, Azelastine, Fexofenadine, Levocabastine, Loratadine, Ebastine, Desloratidine and Mizolastine.

Beta 2 agonists/betamimetics may be either long acting beta 2 Agonists (LABAs) or short acting beta agonists (SABAs). Particularly preferred beta 2 agonists/betamimetics are selected from Bambuterol, Bitolterol, Carbuterol, Clenbuterol, Fenoterol, Formoterol, Hexoprenalin, Ibuterol, Pirbuterol, Procaterol, Reproterol, Salmeterol, Sulfonterol, Terbutalin, Tolubuterol, Olodaterol, and Salbutamol, in particular Olodaterol.

Anticholinergic agents are preferably selected from ipratropium salts, tiotropium salts, glycopyrronium salts, and theophylline, wherein tiotropium bromide is preferred in particular.

Leukotriene modulators are preferably selected from Montelukast, Pranlukast, Zafirlukast, Ibudilast and Zileuton.

JAK inhibitors are preferably selected from Baricitinib, Cerdulatinib, Fedratinib, Filgotinib, Gandotinib, Lestaurtinib, Momelotinib, Pacritinib, Peficitinib, Ruxolitinib, Tofacitinib, and Upadacitinib.

Anti-interleukin antibodies are preferably selected from anti-IL23 antibodies such as Risankizumab, anti-IL17 antibodies, anti-IL1 antibodies, anti-IL4 antibodies, anti-IL13 antibodies, anti-IL-5 antibodies, anti-IL-6 antibodies such as Actemra™, anti-IL-12 antibodies, anti-IL-15 antibodies.

### 8. FORMULATIONS

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, intrasternal, and subcutaneous injection or infusion. Inhalation refers to administration into the patient's lungs whether inhaled through the mouth or through the nasal passages. Topical administration includes application to the skin. The compounds of the invention may be administered via eye drops to treat Sjogren's syndrome.

Suitable forms for administration are for example tablets, capsules, solutions, syrups, emulsions or inhalable powders or aerosols. The content of the pharmaceutically effective compound(s) in each case should be in the range from 0.1 to 90 wt. %, preferably 0.5 to 50 wt. % of the total composition, i.e. in amounts which are sufficient to achieve the dosage range specified hereinafter.

The preparations may be administered orally in the form of a tablet, as a powder, as a powder in a capsule (e.g. a hard gelatin capsule), as a solution or suspension. When administered by inhalation the active substance combination may be given as a powder, as an aqueous or aqueous-ethanolic solution or using a propellant gas formulation.

Preferably, therefore, pharmaceutical formulations are characterized by the content of one or more compounds of formula I, II or III according to the preferred embodiments above.

It is particularly preferable if the compounds of formula I, II or III are administered orally, and it is also particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example kollidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatin capsules. Suitable suppositories may be made for example by mixing with carriers provided for this purpose, such as neutral fats or polyethylene glycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatin and the like. Moreover, lubricants such as magnesium stearate, sodium lauryl sulphate and talc may be used at the same time for the tableting process. In the case of aqueous suspensions, the active substances may be combined with various flavor enhancers or colorings in addition to the excipients mentioned above.

1. A compound of formula I 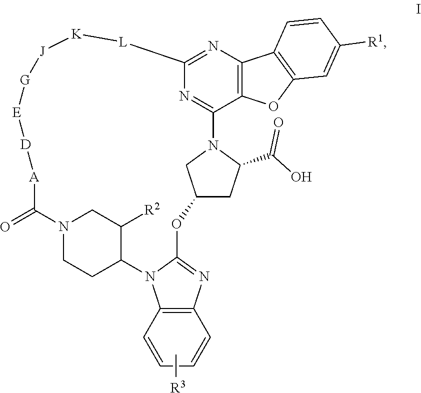
wherein
R¹ is selected from the group consisting of hydrogen, halogen, methyl, ethyl, —CF₃, —CHF₂, —CFH₂ and methoxy,
R² is selected from the group consisting of hydrogen and methyl,
R³ is selected from the group consisting of hydrogen, methyl and halogen,
and wherein
A is selected from the group consisting of —CH₂—, —O—, —CF₂—, —CHF—, —N(CH₃)—, —NH— and —CHCH₃—;
D is selected from the group consisting of —CH₂—, —O—, —CF₂—, —CHF— and —CHCH₃—;
E is selected from the group consisting of —CH₂—, —O—, —C(CH₃)₂—, —CHF—, CF₂— and —CHCH₃—;
G is selected from the group consisting of —NH—, —NCH₃—, —CH₂—, —O—, —CF₂—, —CHF—, —CHCH₃— and —C(CH₃)₂;
J is selected from the group consisting of —CO—, —CH₂—, —O—, —CHF—, —CF₂— and —CHCH₃—;
K is selected from the group consisting of —CH₂—, —CF₂—, —O— or is absent;
L is selected from the group consisting of —CH₂—, —O—, —CHCH₃—, —CHF—, —CF₂— or is absent;
and prodrugs, deuterated analogues and pharmaceutical acceptable salts thereof.

2. The compound of formula I according to claim 1, wherein
R¹ is selected from the group consisting of hydrogen, Cl and F,
R² is selected from the group consisting of hydrogen and methyl,
R³ is selected from the group consisting of hydrogen, methyl, Cl and F,
and wherein
A is selected from the group consisting of —CH₂—, —O—, —CF₂— and —N(CH₃)—,
D is selected from the group consisting of —CH₂—, —O— and —CHCH₃—,
E is selected from the group consisting of —CH₂—, —O— and —C(CH₃)₂—,
G is selected from the group consisting of —NH—, —CH₂—, —O—, —CHCH₃—,
J is selected from the group consisting of —CO—, —CH₂—, —O—, and —CHCH₃—;
K is selected from the group consisting of —CH₂—, —CF₂—, —O— or is absent;
L is selected from the group consisting of —CH₂—, —CF₂— or is absent;
and prodrugs, deuterated analogues and pharmaceutical acceptable salts thereof.

3. The compound of formula I according to claim 1, wherein L is absent,
and prodrugs, deuterated analogues and pharmaceutical acceptable salts thereof.

4. The compound of formula I according to claim 1, wherein L is absent and wherein A is selected from the group consisting of —CH₂— and —CF₂—,
and prodrugs, pharmaceutical acceptable salts thereof or deuterated analogues thereof.

5. The compound of formula I according to claim 1, wherein L is absent and wherein K is CF₂,
and prodrugs, deuterated analogues and pharmaceutical acceptable salts thereof.

6. The compound of formula I according to claim 1, wherein R³ is halogen,
and prodrugs, deuterated analogues and pharmaceutical acceptable salts thereof.

7. The compound of formula I according to claim 6, wherein R³ is Cl or F, and prodrugs, deuterated analogues and pharmaceutical acceptable salts thereof.

8. The compound of formula I according to claim 7, wherein R³ is Cl or F and is located in the 5-position of the benzimidazole moiety, and prodrugs, deuterated analogues and pharmaceutical acceptable salts thereof.

9. The compound of formula I according to claim 1, wherein R¹ is halogen
and prodrugs, deuterated analogues and pharmaceutical acceptable salts thereof.

10. The compound of formula I according to claim 9, wherein R¹ is Cl or F and prodrugs, deuterated analogues and pharmaceutical acceptable salts thereof.

11. The compound of formula I according to claim 1, wherein R¹ is hydrogen,
and prodrugs, deuterated analogues and pharmaceutical acceptable salts thereof.

12. The compound of formula I according to claim 1 which is selected from the group consisting of 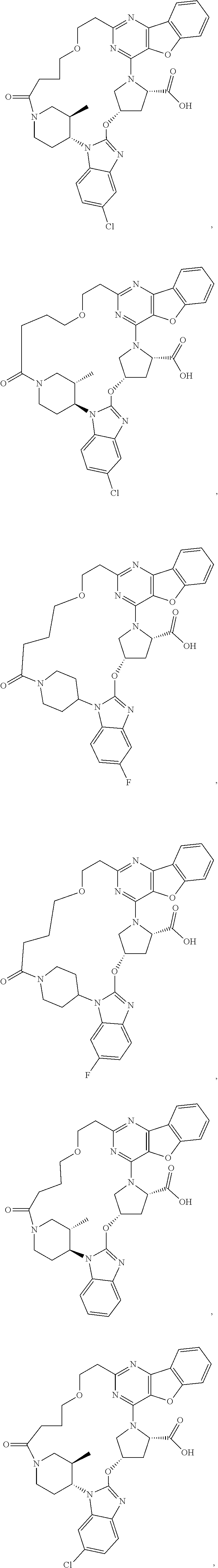 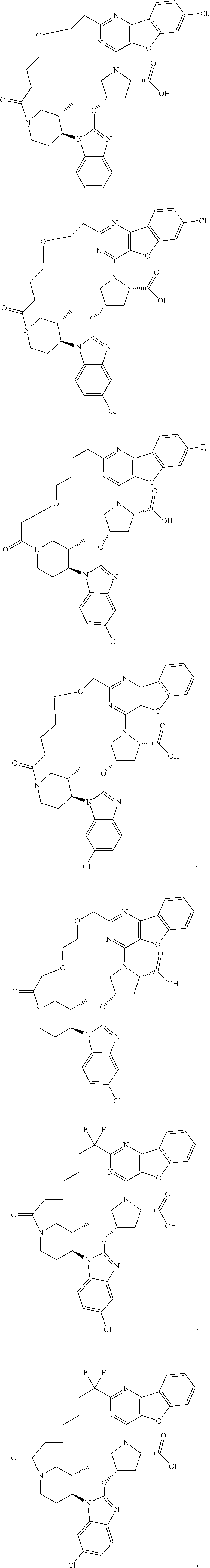 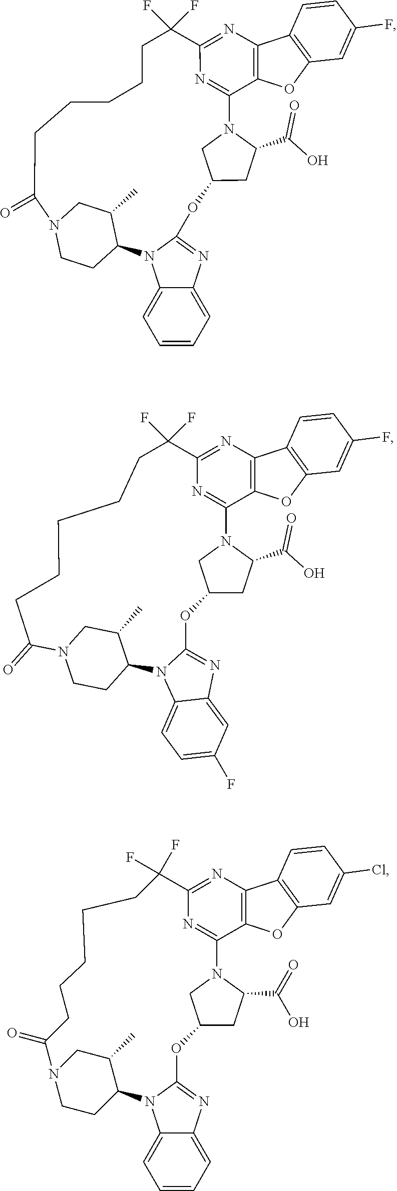 [SMILES: C[C@H]1CN2CC[C@@H]1n1c(nc3ccccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O] 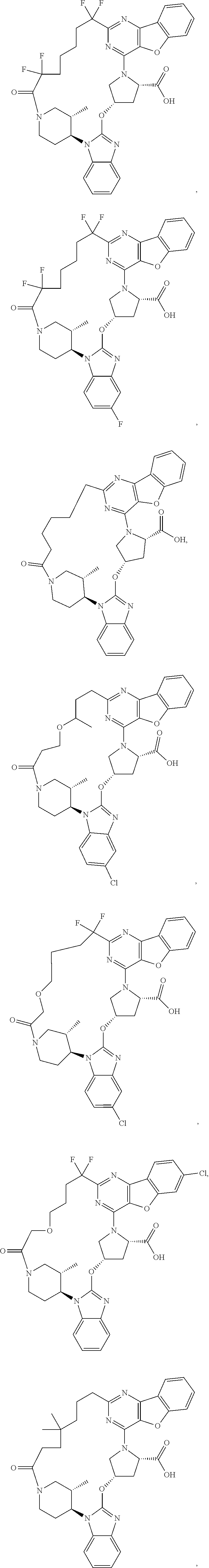 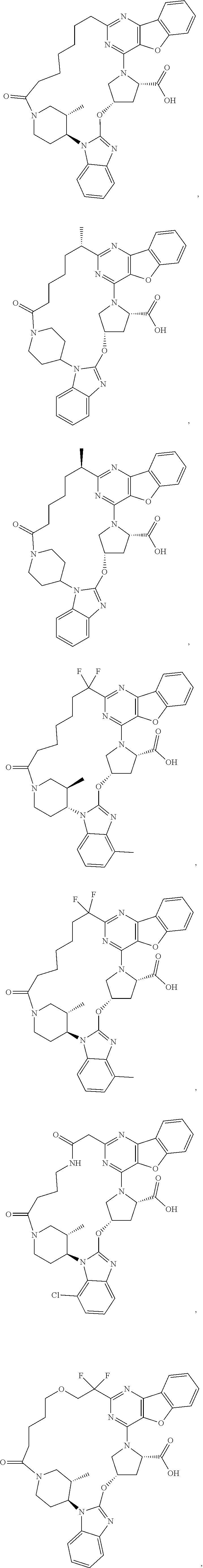 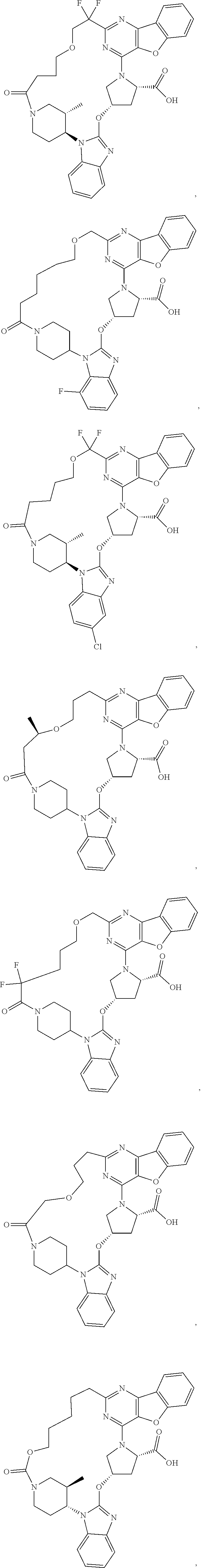 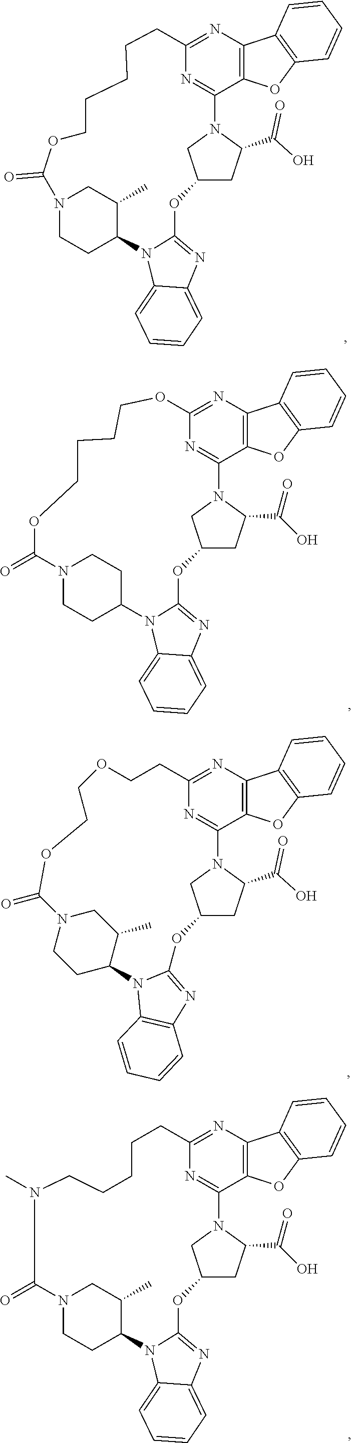
and prodrugs, deuterated analogues and pharmaceutical acceptable salts thereof.

13. The compound of formula I according to claim 1, wherein
A is selected from the group consisting of —CH₂— and —CF₂—,
D and E are each —CH₂—,
G is selected from the group consisting of —CH₂— and —O—,
J is selected from the group consisting of —CH₂— and —O—,
K is selected from the group consisting of —CF₂— and —CH₂—,
and L is absent,
and prodrugs, deuterated analogues and pharmaceutical acceptable salts thereof.

14. The compound of formula I according to claim 13, which is selected from the group consisting of 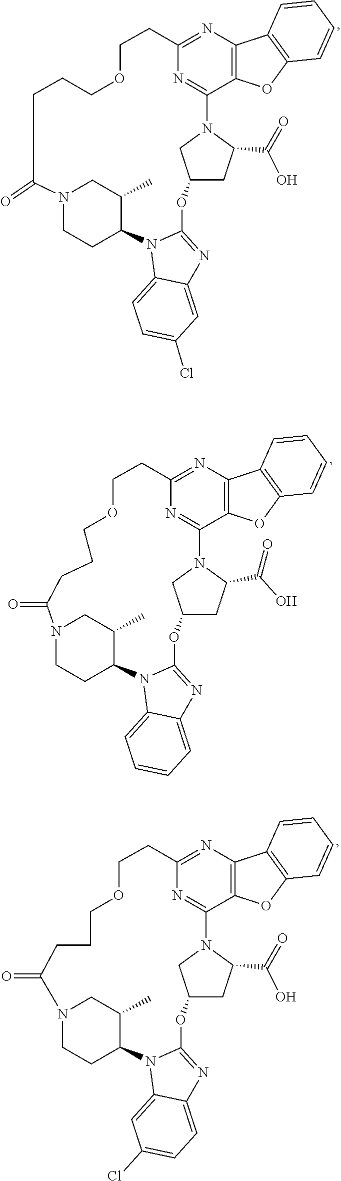 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)CCOCCCC2=O ; C[C@H]1CN2CC[C@@H]1n1c(nc3ccccc31)O[C@H]1C[C@@H](C(=O)O)N(C1)c1nc(nc3c1oc1ccccc13)CCOCCCC2=O ; C[C@H]1CN2CC[C@@H]1n1c(nc3ccc(Cl)cc31)O[C@H]1C[C@@H](C(=O)O)N(C1)c1nc(nc3c1oc1ccccc13)CCOCCCC2=O] 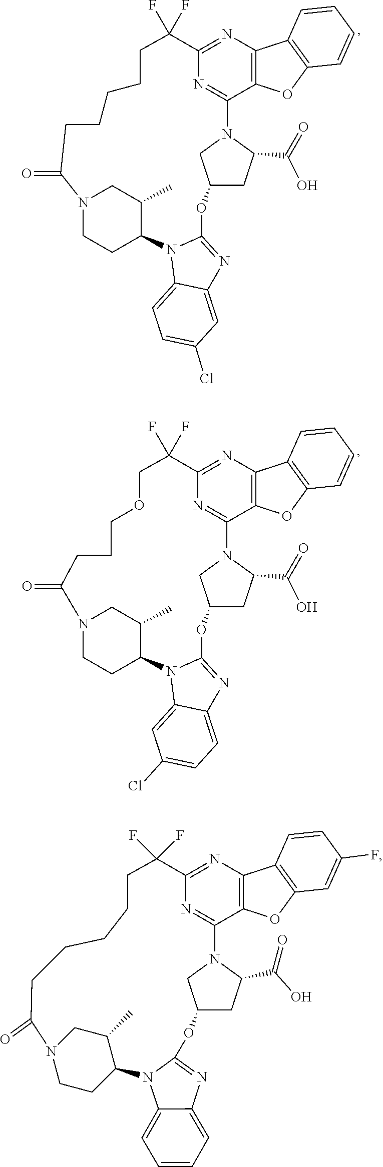 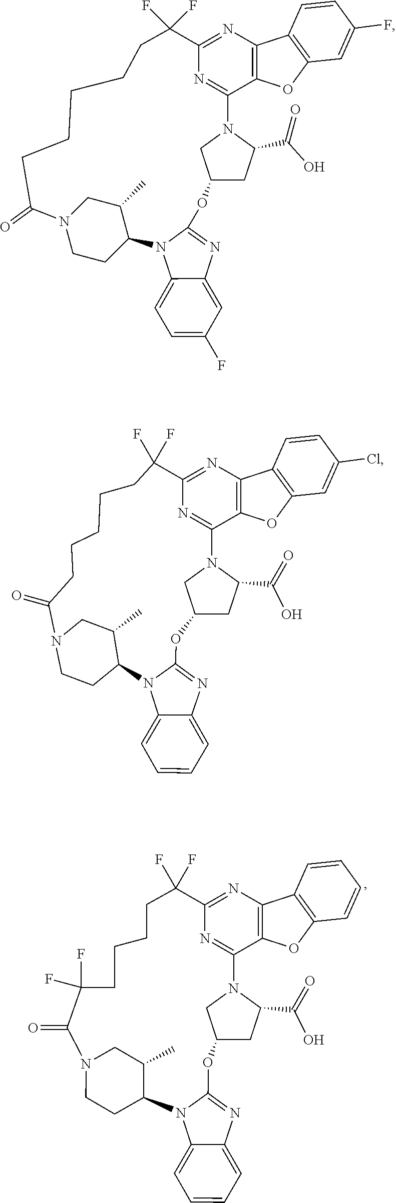 [SMILES: C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1ccccc13)C(F)(F)CCCCC(F)(F)C2=O] 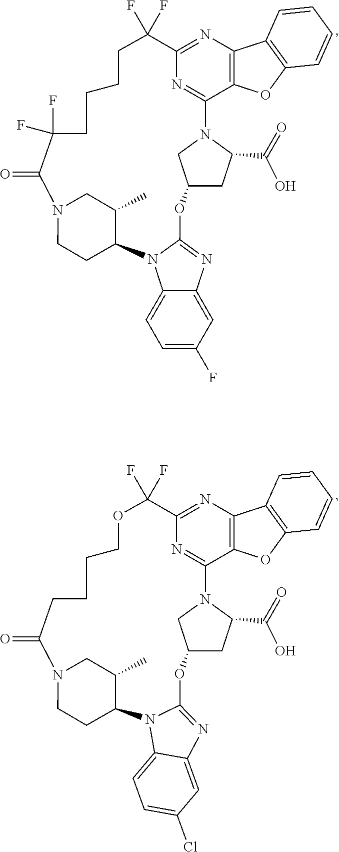 [SMILES: C[C@H]1CN2CC[C@@H]1n1c(nc3cc(F)ccc31)O[C@H]1C[C@@H](C(=O)O)N(C1)c1nc(nc3c1oc1ccccc13)C(F)(F)CCCCC(F)(F)C2=O ; C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O]
and prodrugs, deuterated analogues and pharmaceutical acceptable salts thereof.

15. The compound of formula I according to claim 1, wherein R₂ is methyl, and prodrugs, deuterated analogues and pharmaceutical acceptable salts thereof.

16. The compound of formula II 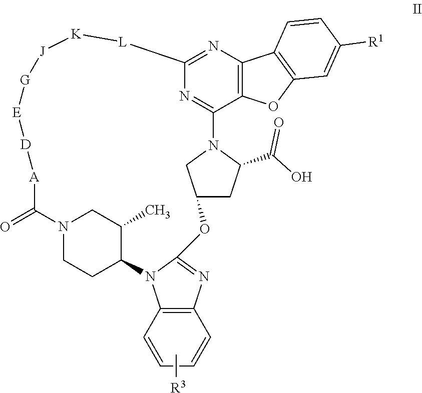
or the compound of formula III 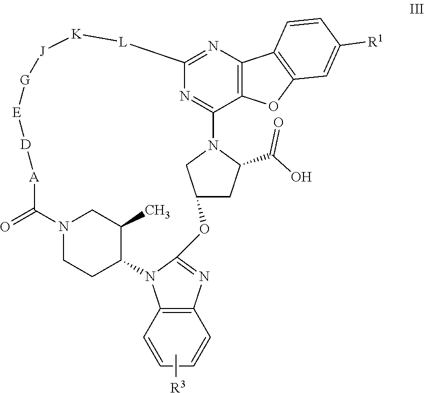
according to claim 15,
and prodrugs, deuterated analogues and pharmaceutical acceptable salts thereof.

17. The compound of formula II 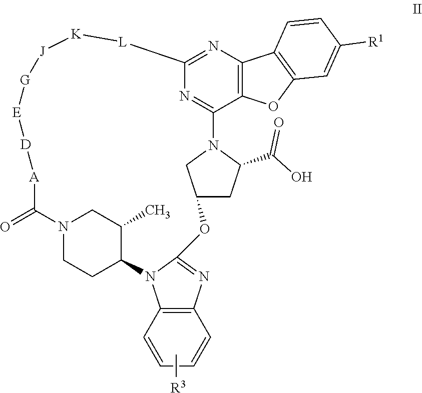
according to claim 16,
and prodrugs, deuterated analogues and pharmaceutical acceptable salts thereof.

18. The compound of formula II or of formula III according to claim 16, wherein L is absent, and prodrugs, deuterated analogues and pharmaceutical acceptable salts thereof.

19. The compound of formula II or of formula III according to claim 16, wherein L is absent and wherein K is CF₂,
and prodrugs, deuterated analogues and pharmaceutical acceptable salts thereof.

20. The compound of formula II or of formula III according to claim 16, wherein R³ is halogen,
and prodrugs, deuterated analogues and pharmaceutical acceptable salts thereof.

21. The compound of formula II or of formula III according to claim 20, wherein R³ is Cl or F,
and prodrugs, deuterated analogues and pharmaceutical acceptable salts thereof.

22. The compound of formula II or of formula III according to claim 21, wherein R³ is Cl or F, and is located in the 5-position of the benzimidazole moiety, and prodrugs, deuterated analogues and pharmaceutical acceptable salts thereof.

23. The compound of formula II or of formula III according to claim 16, wherein R³ is hydrogen, and prodrugs, deuterated analogues and pharmaceutical acceptable salts thereof.

24. The compound of formula II or of formula III according to claim 16, wherein R¹ is halogen,
and prodrugs, deuterated analogues and pharmaceutical acceptable salts thereof.

25. The compound of formula II or of formula III according to claim 24, wherein R¹ is selected from the group consisting of Cl or F,
and prodrugs, deuterated analogues and pharmaceutical acceptable salts thereof.

26. The compound of formula II or of formula III according to claim 16, wherein R¹ is hydrogen,
and prodrugs, deuterated analogues and pharmaceutical acceptable salts thereof.

27. The compound of formula II or of formula III according to claim 16, wherein
A is selected from the group consisting of —CH₂— and —CF₂—,
D and E are each —CH₂—,
G is selected from the group consisting of —CH₂— and —O—,
J is selected from the group consisting of —CH₂— and —O—,
K is selected from the group consisting of —CF₂— and —CH₂—,
and L is absent,
and prodrugs, deuterated analogues and pharmaceutical acceptable salts thereof.

28. The compound of formula II according to claim 27, which is selected from the group consisting of 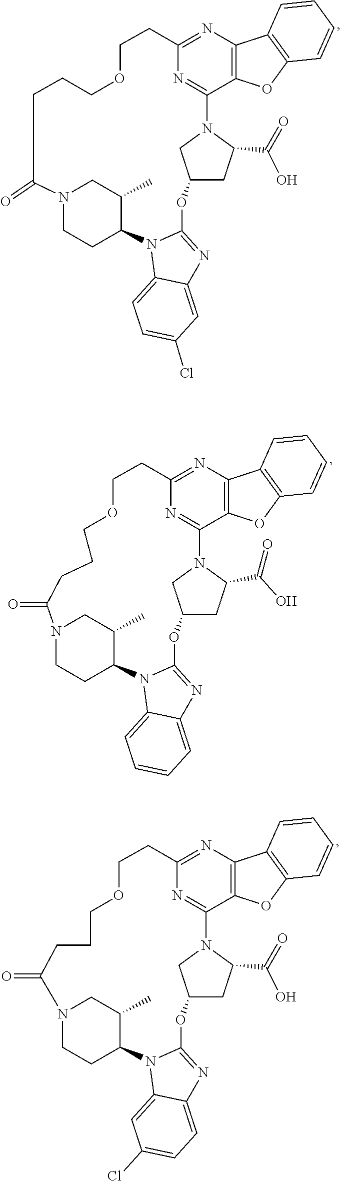 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)CCOCCCC2=O ; C[C@H]1CN2CC[C@@H]1n1c(nc3ccccc31)O[C@H]1C[C@@H](C(=O)O)N(C1)c1nc(nc3c1oc1ccccc13)CCOCCCC2=O ; C[C@H]1CN2CC[C@@H]1n1c(nc3ccc(Cl)cc31)O[C@H]1C[C@@H](C(=O)O)N(C1)c1nc(nc3c1oc1ccccc13)CCOCCCC2=O] 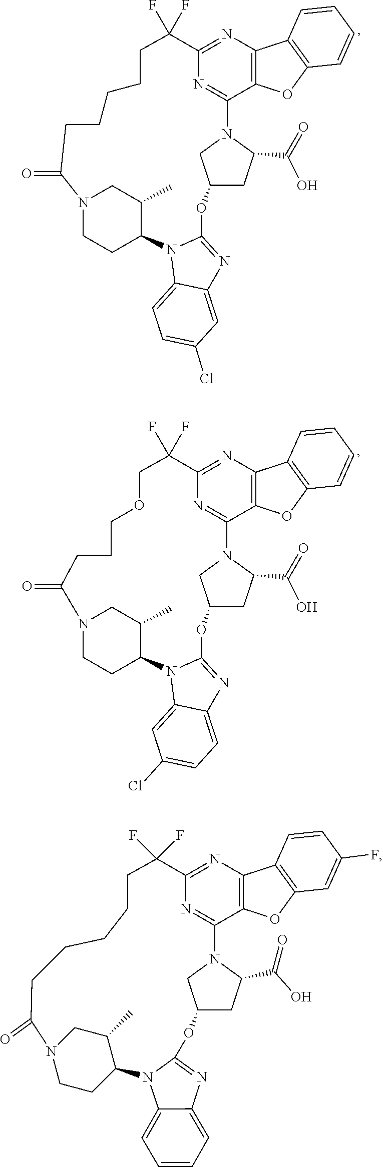 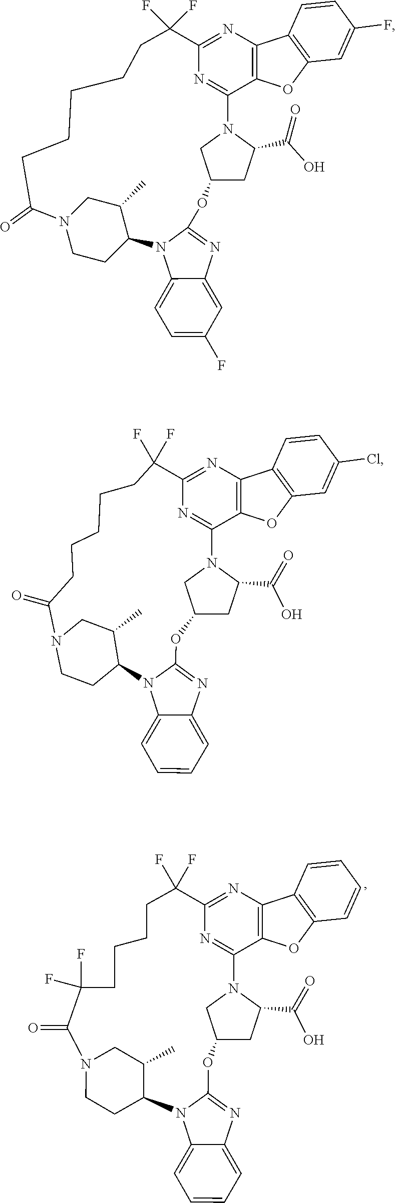 [SMILES: C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1ccccc13)C(F)(F)CCCCC(F)(F)C2=O] 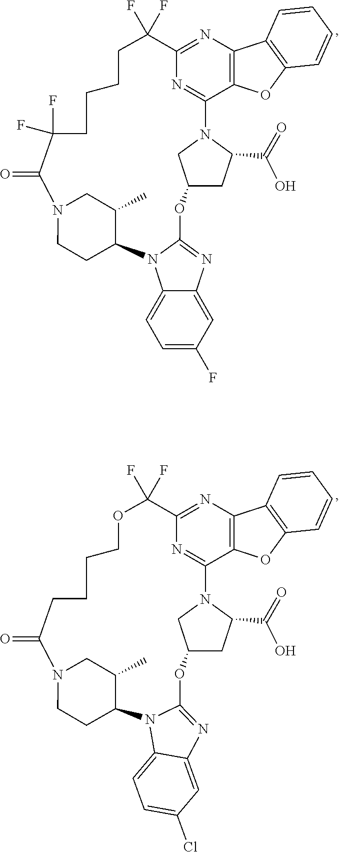 [SMILES: C[C@H]1CN2CC[C@@H]1n1c(nc3cc(F)ccc31)O[C@H]1C[C@@H](C(=O)O)N(C1)c1nc(nc3c1oc1ccccc13)C(F)(F)CCCCC(F)(F)C2=O ; C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O]
and prodrugs, deuterated analogues and pharmaceutical acceptable salts thereof.

29. The compound of formula II or the compound of formula III according to claim 27, wherein
A is selected from the group consisting of —CH₂— and —CF₂—,
D, E and G are each —CH₂—,
J is selected from the group consisting of —CH₂— and —O—,
K is —CF₂—,
and L is absent,
and prodrugs, deuterated analogues and pharmaceutical acceptable salts thereof.

30. The compound of formula II according to claim 29, which is selected from the group consisting of 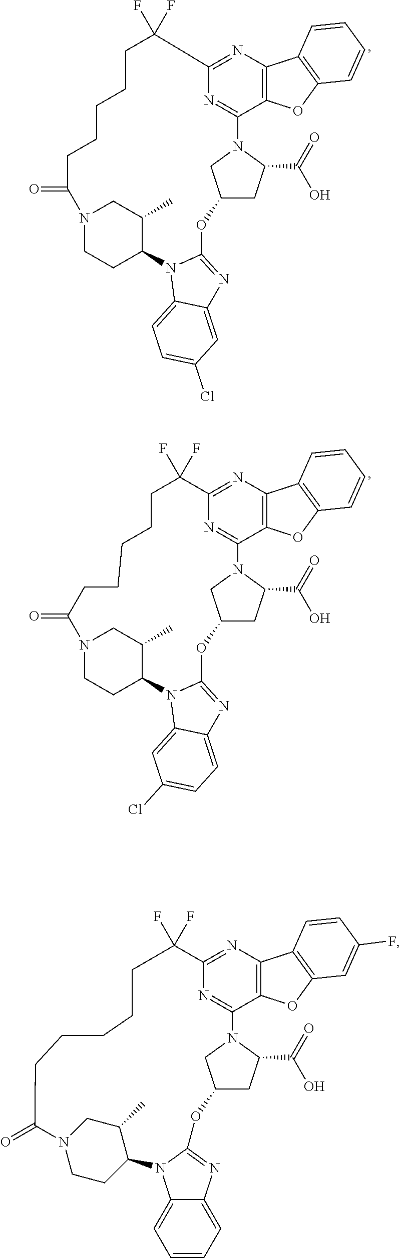 [SMILES: C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)CCCCCC2=O ; C[C@H]1CN2CC[C@@H]1n1c(nc3ccc(Cl)cc31)O[C@H]1C[C@@H](C(=O)O)N(C1)c1nc(nc3c1oc1ccccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(F)ccc13)C(F)(F)CCCCCC2=O] 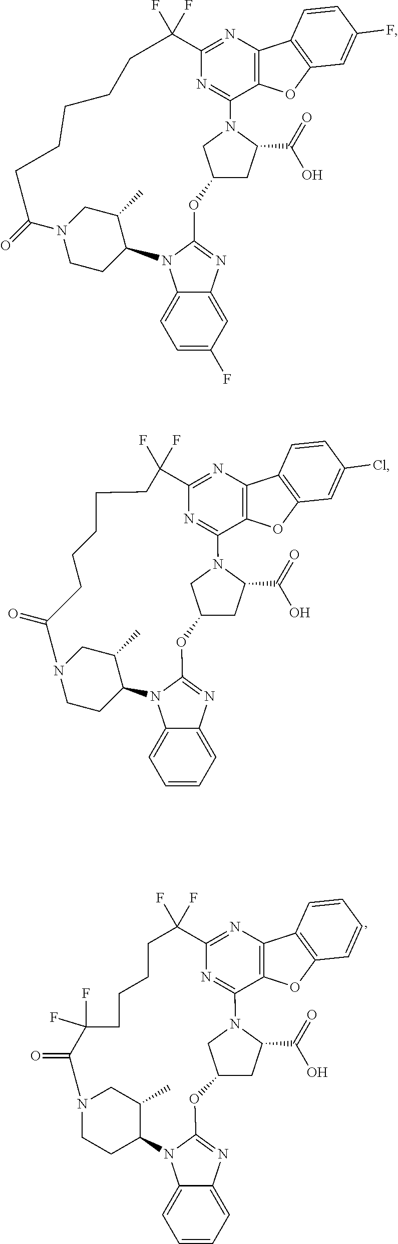 [SMILES: C[C@H]1CN2CC[C@@H]1n1c(nc3cc(F)ccc31)O[C@H]1C[C@@H](C(=O)O)N(C1)c1nc(nc3c1oc1cc(F)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1cc(Cl)ccc13)C(F)(F)CCCCCC2=O ; C[C@H]1CN2CC[C@@H]1n1c(nc3ccccc31)O[C@H]1C[C@@H](C(=O)O)N(C1)c1nc(nc3c1oc1ccccc13)C(F)(F)CCCCC(F)(F)C2=O] 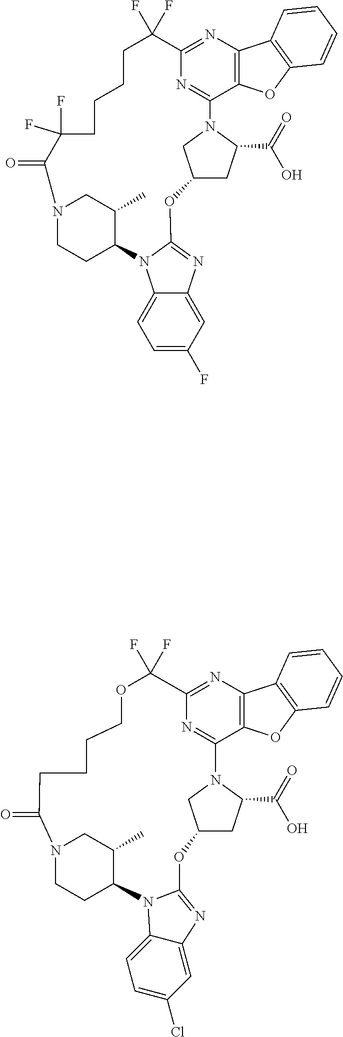 [SMILES: C[C@H]1CN2CC[C@@H]1n1c(nc3cc(F)ccc31)O[C@H]1C[C@@H](C(=O)O)N(C1)c1nc(nc3c1oc1ccccc13)C(F)(F)CCCCC(F)(F)C2=O ; C[C@H]1CN2CC[C@@H]1n1c(nc3cc(Cl)ccc31)O[C@H]1C[C@@H](C(=O)O)N(C1)c1nc(nc3c1oc1ccccc13)C(F)(F)OCCCCC2=O]
and prodrugs, deuterated analogues and pharmaceutical acceptable salts thereof.

31. An intermediate compound of
formula (A-I) 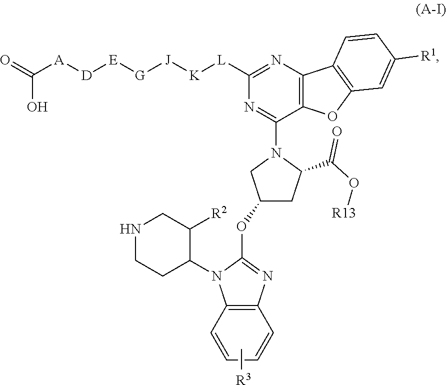
wherein R¹, R², R³, A, D, E, G, J, K and L are defined as in claim 1 and
wherein R¹³ is selected from the group consisting of hydrogen, methyl, ethyl and tert-butyl,
of formula (A-II), 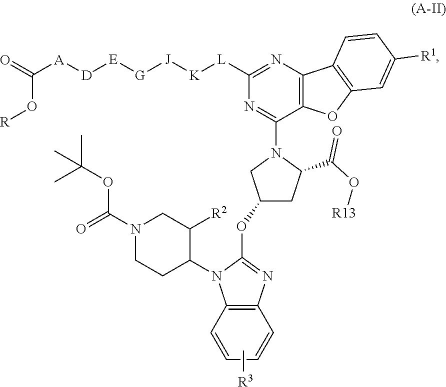
wherein R¹, R², R³, A, D, E, G, J, K and L are defined as in claim 1 above mentioned and
wherein R¹³ is selected from the group consisting of hydrogen, methyl, ethyl and tert-butyl, and wherein R is either hydrogen or a protecting group selected from the group consisting of tert-butyl, methyl, ethyl and benzyl,
of formula (B-I) 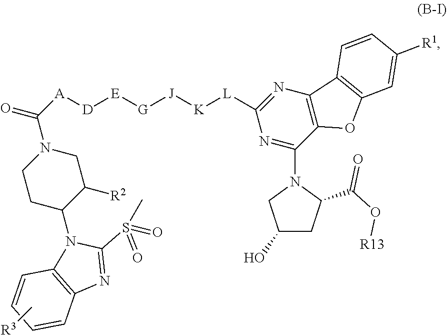
wherein R¹, R², R³, A, D, E, G, J, K and L are defined as in claim 1 and
wherein R¹³ is selected from the group consisting of hydrogen, methyl, ethyl and tert-butyl, or
of formula (B-II) 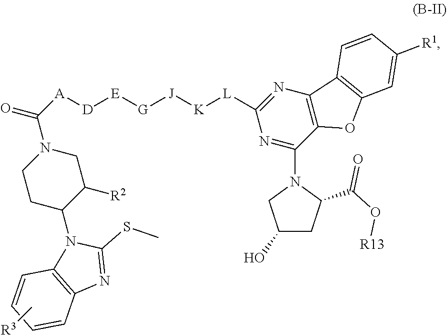
wherein R¹, R², R³, A, D, E, G, J, K and L are defined as in claim 1 and wherein R¹³ is selected from the group consisting of hydrogen, methyl, ethyl and tert-butyl.

32. A method of treating in a patient a disease that can be treated by the inhibition of cGAS, said method comprising administering to the patient a compound of formula I according to claim 1.

33. A method of treating a disease in a patient, said method comprising administering to the patient a compound of formula I according to claim 1, wherein the disease is selected from the group consisting of systemic lupus erythematosus (SLE), interferonopathies, Aicardi-Goutières syndrome (AGS), COPA syndrome, familial chilblain lupus, age-related macular degeneration (AMD), amyotrophic lateral sclerosis (ALS), retinopathy, glaucoma, diabetes, obesity, inflammatory bowel disease (IBD), chronic obstructive pulmonary disease (COPD), Bloom's syndrome, dermatomyositis, Sjogren's syndrome, Parkinsons disease, heart failure, cancer, aging, muscle disorders, sepsis, rheumatoid arthritis, osteoarthritis, COVID-19, systemic sclerosis (SSc), non-alcoholic steatotic hepatitis (NASH), interstitial lung disease (ILD), progressive fibrosing interstitial lung disease (PF-ILD), and idiopathic pulmonary fibrosis (IPF).

34. The method according to claim 33, wherein the disease is selected from the group consisting of systemic lupus erythematosus (SLE), interferonopathies, Aicardi-Goutières syndrome (AGS), COPA syndrome, familial chilblain lupus, dermatomyositis, age-related macular degeneration (AMD), amyotrophic lateral sclerosis (ALS), inflammatory bowel disease (IBD), chronic obstructive pulmonary disease (COPD), Bloom's syndrome, Sjogren's syndrome, rheumatoid arthritis and Parkinsons disease.

35. The method according to claim 33, wherein the disease is selected from the group consisting of systemic sclerosis (SSc), non-alcoholic steatohepatitis (NASH), interferonopathies, interstitial lung disease (ILD), progressive fibrosing interstitial lung disease (PF-ILD), and idiopathic pulmonary fibrosis (IPF).

36. A method of treating a disease in a patient, said method comprising administering to the patient a compound of formula I according to claim 1, wherein the disease is selected from the group consisting of age-related macular degeneration (AMD), retinopathy, glaucoma, diabetes, obesity, aging, muscle disorders, sepsis, osteoarthritis, heart failure, COVID-19/SARS-CoV-2 infection, renal inflammation, renal fibrosis, dysmetabolism, vascular diseases, cardiovascular diseases and cancer.

37. A pharmaceutical composition comprising the compound of formula I according to claim 1 and optionally one or more pharmaceutically acceptable carriers and/or excipients.

38. The pharmaceutical composition according to claim 37 in combination with one or more active agents selected from the group consisting of anti-inflammatory agents, anti-fibrotic agents, anti-allergic agents/anti-histamines, bronchodilators, beta 2 agonists/betamimetics, adrenergic agonists, anticholinergic agents, methotrexate, mycophenolate mofetil, leukotriene modulators, JAK inhibitors, anti-interleukin antibodies, non-specific immunotherapeutics such as interferons or other cytokines/chemokines, cytokine/chemokine receptor modulators, toll-like receptor agonists, immune checkpoint regulators, an anti-TNF antibody, Adalimumab, an anti-BAFF antibody, Belimumab and Etanercept,
and optionally one or more pharmaceutically acceptable carriers and/or excipients.

39. The pharmaceutical composition according to claim 37 in combination with one or more anti-fibrotic agents selected from the group consisting of Pirfenidon and Nintedanib, and optionally one or more pharmaceutically acceptable carriers and/or excipients.

40. The pharmaceutical composition according to claim 37 in combination with one or more anti-inflammatory agents selected from the group consisting of NSAIDs and corticosteroids, and optionally one or more pharmaceutically acceptable carriers and/or excipients.

41. The pharmaceutical composition according to claim 37 in combination with one or more active agents selected from the group consisting of bronchodilators, beta 2 agonists/betamimetics, adrenergic agonists and anticholinergic agents, and optionally one or more pharmaceutically acceptable carriers and/or excipients.

42. The pharmaceutical composition according to claim 37 and one or more anti-interleukin antibodies selected from the group consisting of anti-IL-23 antibodies, Risankizumab, anti-IL-17 antibodies, anti-IL-1 antibodies, anti-IL-4 antibodies, anti-IL-13 antibodies, anti-IL-5 antibodies, anti-IL-6 antibodies, Tocilizumab, anti-IL-12 antibodies and anti-IL-15 antibodies.
